(19) United States
(12) Patent Application Publication
(10) Pub. No.: US 2004/0194344 A1
(43) Pub. Date: Oct. 7, 2004

(54) User-customizable insoles for footwear and method of customizing insoles

(76) Inventor: Anthony G. Tadin, Woodside, CA (US)

Correspondence Address: LAW OFFICE OF BRIAN R. RAYVE, P.O. Box 11586, Salt Lake City, UT, 84147-0586, US

(21) Appl. No.: 10/818,880

(22) Filed: Apr. 5, 2004

Related U.S. Application Data
(63) Continuation of application No. 60/460,800, filed on Apr. 5, 2003.

(51) Int. Cl. A43B 13/18; A43B 13/38; A43B 23/00; A43B 7/16; A61F 5/14

(52) U.S. Cl. 36/44; 36/173; 36/178

(57) ABSTRACT

A user-customizable insole for insertion into foot wear in place of a conventional removable insole. The insole includes a cushioning upper insole and a semi-rigid yet resiliently flexible base insole used with insole adjustment devices. The upper insole is made of a resilient cushioning material such as foam. The base insole is made from relatively thin plastic with upwardly formed arch supports defining corresponding elevation curves between the base insole and a foot bed or inside bottom of the footwear to permit downward flexing of the arch supports. The arch supports are of a predetermined flexibility by controlling the material thickness and/or adding perforations therethrough. An upwardly formed perimeter wall extends around a heel portion forwardly along respective medial and lateral sides of the base insole tapering away at about a forefoot portion of the base insole defining a heel cup. The upper and lower insoles may be two-thirds length extending from the heel to the behind the toes of the wearer's foot or of full length extending from the heel to ends of the toes of the wearer's foot. The two-thirds length base insole includes a first metatarsal head extension which extends forwardly to support the first metatarsal head of the wearer's foot. The upper insole and the base insole are affixed together at a mid-portion of each with respective forefoot and heel portions unaffixed to permit access for adjustment devices to be added and removed. The adjustment devices may include a first metatarsal head extension boot, forefoot and heel wedges, resilient arch support pads, heel lifts, and resilient heel cushion pads which are self-sticking to the insoles to adjustably position the wearer's foot within the foot wear.

This application claims priority of my copending U.S. Provisional Patent Application No. 60/460,800 filed Apr. 5, 2003.

### BACKGROUND OF THE INVENTION

1. Field

The present invention relates generally to insoles and foot support devices for footwear, and more particularly to removable insoles having a cushioning upper insole supported by a more rigid base insole.

2. State of the Art

Footwear takes many forms including dress shoes, casual shoes, athletic shoes, sandals, and the like. The main functions of footwear are to protect the wearer's feet from contact with the ground, to keep the wearer's feet warm and dry, to provide enhanced performance to the wearer's feet such as for use in athletics, and to provide a stylish look. The footwear must do this while providing adequate comfort and support to the feet.

Contoured insoles are commonly used in footwear such as athletic shoes to provide enhanced comfort and support and are becoming more common for use in other types of footwear. Insoles are inserted into the footwear overlying a foot bed or inside bottom (hereinafter referred to as “inside bottom”) of the footwear. The wearer's foot rests directly on the insole which typically has an upper surface contoured to the sole or plantar surface of the foot to provide cushioning and to support the arches and bones of the wearer's foot. Cushioning generic insoles are produced at shoe factories as a standard component of athletic shoes. Such generic insoles typically have a minimally contoured upper surface with the most prominent contour being an upwardly curved longitudinal medial arch support. While these generic insoles come in the particular size and width for the foot size of the athletic shoe, they are not custom fitted to cushion and support the particular wearer's foot. Many of these generic insoles are removable for separate washing or for replacement upon wearing out. Some athletic shoes come with self-adhesive arch supports which are for placement on the upper surface of the insole to raise the contour of the longitudinal medial arch support to provide greater support thereto. The wearers of athletic shoes, and for that matter other footwear, are quite limited in what adjustments they can make to the footwear by themselves without the assistance of a physician, typically limited to the arch supports. Yet, improperly fitting footwear is the cause of many foot, leg, and back problems. Even normal everyday non-athletic activities such as walking or running involves repetitive impacts of the wearer's feet against the footwear as the ground is contacted. These impacts move from the feet to the ankles, along the legs to the spine potentially leading to problems along the entire course. The lack of sufficient cushion and support in the footwear increases the risk of such problems.

The leg and foot are a complex system of bones, muscles, and tendons which interact in a precise manner to produce a walking gait or stride. Lower bones of the leg include the tibia and the fibula bones which extend downwardly from the knee to the ankle. A plurality of tarsal bones are disposed below the tibia and fibula, including an ankle or talus bone positioned directly therebelow, three cuneiform bones, a cuboid bone, and a navicular bone. The cuboid and the navicular bones are positioned below and forwardly of the talus bone. A heel or calcaneus bone is positioned below and rearwardly of the talus bone forming the heel of the foot. The talus bone rests on the calcaneus bone. First through fifth metatarsal bones extend forwardly from the cuneiform bones and the cuboid bone. The cuneiform bones are connected to the first, second and third metatarsals. The cuboid bone interconnects the fourth and fifth metatarsals to the calcaneus bone. The navicular bone interconnects the cuneiform bones to the talus bone and rests on the cuboid bone. Five phalanges extend forwardly from the metatarsal bones which make up the five toes of the foot.

The bones of the feet form upwardly curved arches at the plantar surface of the foot to support the weight of the person on the relatively small area of the foot's plantar area. A transverse arch or metatarsal arch is formed by the first through fifth metatarsal heads. An inner longitudinal arch or medial arch is formed by the first through third metatarsal bones, the cuneiform bones, the navicular bone, the talus bone, and the calcaneus bone. An outer longitudinal arch or lateral arch is formed by the fourth and fifth metatarsal bones, the cuboid bone, and the calcaneus bone. The metatarsal arch, the medial arch, and the lateral arch provide a structure which allows the feet to support the weight of the person on the relatively small area of the plantar surface.

The foot is a complex mechanism which is designed to permit hinging and rotational motions while disposed in certain positions of the normal walking or running stride while simultaneously restricting other motions. For example, the movement of the talus bone relative to the tibia and the fibula bones is such that the entire foot may be pivoted vertically upward and downward to raise and lower the toes of the foot. The talus bone is connected to the tibia and fibula bones in such a way that as the person's leg rotates about a vertical axis during the walking or running stride, the talus bone and thus the entire foot rotates inwardly and outwardly with the leg. The talus and the calcaneus bones move relative to one another about a subtalar joint. A hinge axis of the subtalar joint extends upwardly and forwardly at an angle of about forty-five degrees from horizontal, and extends forwardly and inwardly at an angle of about fifteen degrees from forward. A midtarsal joint comprise two separate joints, a talo-navicular joint and a calcaneal-cuboid joint. As the talus bone rotates inwardly about the vertical axis of the leg as the front part of the leg is rotated toward the center of the wearer's body, there is a corresponding rotation of the calcancus about a horizontal axis. An opposite or outward rotation of the talus causes a corresponding rotation of the calcaneus in the opposite direction.

During the normal walking and running strides the person's pelvis moves on a transverse plane and the femur and the tibia rotate about the vertical axis of the leg as the legs swing forwardly and rearwardly. This causes alternating pronating and supinating of the foot with the midtarsal joints alternately locking and unlocking during the walking or running stride. When the particular leg moves forwardly there is an inward rotation of the leg and the talus bone about the vertical axis of the leg resulting in the midtarsal joint unlocking. This unlocking of the midtarsal joint allows the foot ahead of the talus bone to move from a neutral position to a pronated position rotated a few degrees inwardly. This results in a lowering of the longitudinal arch of the foot to help absorb the impact as the heel below the calcaneus bone contacts the ground. As the particular leg moves rearwardly, the leg and talus rotate outwardly about the vertical axis of the leg resulting in the midtarsal joint locking. This locking of the midtarsal joint causes the foot ahead of the talus bone to become part of a semi-rigid lever with the tibia and fibula in the supinated position of the foot to allow push-off of a ball area of the foot under the metatarsal heads and phlanges from the ground. The midtarsal joint thus functions to keep the plantar surface at the midportion of the foot engaged with the ground so as to momentarily remain generally horizontal to the ground. The motion produced by the locking and locking of the subtalar and midtarsal joints to produce pronation and supination is not perpendicular to any one plane of the body and is therefore a complex tri-planar motion.

While pronating and supinating of the foot is normal and necessary for walking and running, either to the excess may cause problems to develop within the feet, ankles, legs, knees, or back. For example, in overpronation the foot is excessively rotated inwardly about the vertical axis of the leg producing an exaggerated hinging motion of the foot from side to side and producing the appearance of a low medial longitudinal arch. The person's weight is placed on the forefoot and heel of the foot further than normal toward the medial side which can lead to lower back, knee, shin, ankle, and foot problems. Conversely, the supinated or underpronated foot is excessively rotated outwardly about the vertical axis of the leg such that there is little inward rotation as the leg moves from the heel to forefoot. The foot appears to have a high medial longitudinal arch and a more curved appearance on the medial side of the foot than is normal. The foot and leg is constantly in the semi-rigid lever mode and does not readily absorb shocks. The foot rigidly rolls on the lateral side with little weight applied to the medial longitudinal arch which produces excessive shock that can lead to lower back, knee, shin, ankle, and foot problems.

Special contoured insoles, arch supports, and medical orthotic devices have been designed to correct the above-described foot problems. Such devices, particularly the orthotic devices, are typically designed to provide support to the plantar surface of the foot so as to properly position the various bones of the foot wherein the subtalar and midtarsal joints are initially positioned properly. The orthotic device controls the motion and initial position of the bones and joints of the foot so that the movements of the hip, leg, and foot during the walking and running stride are correct. When the bones and joints of the foot are initially positioned correctly and their motion is controlled, the entire walking and running stride from the initial strike of the heel on the ground to the final push off from the toe of the foot is properly coordinated.

Various devices and methods have been developed to measure the contours of the foot to facilitate making orthotic devices which are custom fit to the wearer's foot. For example, in U.S. Pat. Nos. 6,493,958 and 6,430,831 issued respectively to the present inventor and to Sundman are disclosed methods and apparatuses for measuring the plantar contour of the foot using a foam impression block and a carrier. In U.S. Pat. No. 6,205,230 issued to Sundman et al. Is disclosed an optical contour digitizer and method of use for optically scanning the plantar contour of the foot. Finally, in U.S. Pat. Nos. 5,941,835 issued to Sundman; 5,689,446 issued to Sundman et al.; 5,640,779 and 4,876,758 both issued to Rolloff et al.; 4,517,696 and 4,449,264 both issued to Schartz; 4,510,636 issued to Phillips; and 4,454,618 issued to Curchod are disclosed various methods and apparatuses for measuring the plantar contour which utilize pluralities of vertically-oriented pins. Some of these pin-type apparatuses also form the insoles to the measured plantar contour directly from the pins. All such devices and methods developed to measure the contours of the plantar surface may be used to allow determination of how the insoles and adjustment devices should be designed and what adjustment devices should be utilized in the present invention. Thus, the disclosures of all of the patents listed in this paragraph are herein incorporated by reference.

While the various contoured insoles, arch supports, medical orthotic, and other devices are generally adequate for the purposes for which they were designed, none are designed to allow the individual wearer to adequately adjust for the feet. The generic contoured insoles and arch supports provide none or only minimal customization for the wearer's feet, typically only adjustment for the longitudinal medial arch. Specially fitted arch supports and medical orthotic devices must be designed for and fitted to the wearer's feet by a podiatrist or other skilled professional. There is a need for an insole which allows the untrained wearer to adjust the foot position using standard insole adjustment devices.

### SUMMARY OF THE INVENTION

The present invention are user-customizable insoles for placement onto the inside bottom of footwear to support the plantar surface of a wearer's foot, which includes a cushioning upper insole and a base insole, insole customizing kits, and a method for customizing insoles for a wearer's foot. The insoles extend from at least about the sulcus cavity ahead of metatarsal bones to underneath the calcaneus bone of the wearer's foot.

The cushioning upper insole is relatively soft and shaped to fit into the footwear. The upper insole has an upper surface adapted to engage the plantar surface of the wearer's foot and a lower surface. A forefoot portion is positionable underneath the metatarsal bones. A heel portion is positionable underneath the calcaneus bone. A mid-portion of the base insole interconnects the forefoot and heel portions. The upper insole may be of a two-thirds length where the forefoot portion begins at a forward edge disposed adjacent the sulcus cavity of the wearer's foot. Alternatively, the upper insole may be of the two-thirds length with a first metatarsal head extension which extends from the forward edge under the first metatarsal head of the wearer's foot. Further alternatively, the upper insole may be of a full length with a toe portion positionable underneath phalanges of the wearer's foot. The upper insole may be of a monolithic construction comprising a body made of a flexible resilient cushioning material. Alternatively, the upper insole may be of a flexible sandwiched construction comprising a flexible cloth sheet bonded to a body made of a flexible resilient cushioning material. Further alternatively, the upper insole may be of a cloth construction comprising a flexible cloth sheet with the bottom surface covered with an adhesive layer. Yet further alternatively, the upper insole may be of a semi-rigid sandwiched construction comprising a flexible cloth sheet bonded to a semi-rigid contoured foam body. Finally, the upper insole may be of a thermoformable construction comprising a body made of a thermoformable material which uses body heat from the wearer's foot to semi-permanently mold to the contours of the plantar surface.

The base insole is semi-rigid and resiliently flexible, and shaped to fit into the footwear. The base insole has an upper surface which is contoured to provide support to the plantar surface of the wearer's foot and a lower surface adapted to engaging the inside bottom of the footwear. A forefoot portion is positionable underneath the metatarsal bones. A heel portion is positionable underneath the calcaneus bone of the foot. A mid-portion of the base insole which interconnects the forefoot and heel portions. The base insole may be of a two-thirds length where the forefoot portion begins at a forward edge disposed adjacent the sulcus cavity. Alternatively, the base insole may be of a two-thirds length with a first metatarsal head extension which extends from the forward edge under the first metatarsal head of the wearer's foot. Further alternatively, the base insole may be of a full length with a toe portion positionable underneath phalanges of the wearer's foot with about a front most one-third of the base insole being of a different thickness than a remaining portion thereof

A preferred base insole is relatively thin and of a generally uniform thickness. The base insoles includes a tapered perimeter wall which extends around the heel portion and forwardly along respective medial and lateral sides of the base insole defining a heel cup, the support wall tapering away at about the forefoot portion of the base insole. The heel has a flat or contoured bottom and is of a depth sufficient to accept at least one intrinsic heel adjustment device therein. The base insole includes at least one integral foot support chosen from the group consisting of a metatarsal arch support, a cuboid support, a medial longitudinal arch support, a lateral arch support, and a medial longitudinal ligament support. At least one of the upwardly curved metatarsal arch support, the cuboid support, the medial longitudinal arch support, the lateral arch support, and the medial longitudinal ligament support has a corresponding upwardly curved elevation curve therebelow. Each elevation curve defines a corresponding space with the base insole such that the foot support may flex downwardly under a predetermined foot pressure to reduce shock and provide selected support to the wearer's foot. The predetermined foot pressure of each foot support is controlled by using an insole strength modifying feature such as adding one or more perforations, open-ended slots, thinning the base insole material, adding external material to selectively strengthen the slots, and regional changes to mechanical properties of material of the base insole. A downwardly extending lateral pivot has a lowermost portion which is slightly above or even with a bottom plane of the base insole. An integrally molded or affixed heel support extends downwardly from the heel portion. The heel support has a relatively flat bottom which engages the inside bottom of the footwear to stabilize the heel of the wearer's foot within the footwear. The mid-portion of the upper insole is affixed to the mid-portion of the base insole such that the heel portion and forefoot portion of the upper insole may be raised from the base insole to add and remove adjustment devices.

The insole customizing kits include one or more insole adjustment devices adapted to be used with the insole for adjusting positioning of the foot within the foot wear. At least one insole adjustment device is an intrinsic or extrinsic insole adjustment device comprising a first metatarsal head extension boot, a forefoot medial wedge, a forefoot lateral wedge, an elongate medial longitudinal wedge, an elongate lateral longitudinal wedge, a bi-planar rear post, a heel lift, a resilient metatarsal arch support pad, a resilient medial longitudinal arch support pad, a medial half-heel wedge, a lateral half-heel wedge, a full-width heel wedge, a resilient contoured foam heel pad, a resilient pivot pad, a resilient undersurface pad, a contoured gel heel pad, and a custom positionable toe grip. Each adjustment device preferably includes a peel-off backing sheet which exposes an adhesive layer on the adjustment device to secure the adjustment device to one or both of the upper insole and the base insole. Other insole adjustment devices include a metatarsal arch spring support and a lateral cuboid spring support each of which include a respective compression spring one end of which is secured to a corresponding of the elevation curves to respectively back-fill the open space therebelow to provide extra support therefor.

The method for customizing insoles of footwear for a wearer's foot comprises the steps of: 1) providing an article of footwear; 2) providing an insole adapted for placement onto an inside bottom of the footwear to support a plantar surface of the wearer's foot, the insole having a cushioning upper insole with a forefoot portion, a mid-portion, and a heel portion, and a base insole shaped to fit to the footwear having a forefoot portion, a mid-portion, and a heel portion with a heel cup; 3) providing a plurality of insole adjustment devices each adapted to correct a foot position abnormality and support the wearer's foot within the footwear in a corrected position; 4) selecting at least one of the insole adjustment devices to correct the foot position abnormality of the wearer's foot and support the foot in the corrected position; 5) placing the selected adjustment devices into respective desired corrective positions to the insole; and 6) inserting the insole with the selected adjustment devices in the corrective positions into the article of footwear. The adjustment devices are preferably first metatarsal head boots, forefoot medial wedges, forefoot lateral wedges, medial longitudinal wedges, lateral longitudinal wedges, bi-planar rear posts, heel lifts, resilient metatarsal arch support pads, resilient longitudinal arch support pads, heel medial half-wedges, heel lateral half wedges, heel full wedges, resilient contoured heel pads, resilient pivot pads, resilient undersurface pads, heel gel pads, and spring insole adjustment devices.

These and other features and advantages of the present invention will become apparent to those skilled in the art upon a reading of the following detailed description when taken in conjunction with the drawings wherein there is shown and described an illustrative embodiment of the invention.

### THE DRAWINGS

The best mode presently contemplated for carrying out the invention is illustrated in the accompanying drawings, in which:

FIG. 1 is an exploded perspective view of a first embodiment user-customizable insole as used with a first version insole customizing kit, the insole including a first version upper insole of two-thirds length and a first version base insole of two-thirds length, the first version insole customizing kit including insole adjustment devices comprising a first version first metatarsal head extension boot, a bi-planar rear post, a heel lift, a metatarsal arch support pad, and a longitudinal arch support pad;

FIG. 2, a top plan view of the first version base insole showing the contours of an upper surface for supporting the wearer's foot and a first metatarsal head extension;

FIG. 3, a side elevational view of the first version base insole from the lateral side, showing a flat heel and forefoot portion with a forwardly extending first metatarsal support extension tab at a lower surface of the base insole defining a bottom plane, a lateral longitudinal arch support and a cuboid support curving upwardly from the plane, and a pivot disposed just above the bottom plane;

FIG. 4, a side elevational view of the first version base insole from the medial side, showing a medial longitudinal arch support curving upwardly from the bottom plane;

FIG. 5, an exploded perspective view of the first metatarsal head extension boot which slips over the first metatarsal support extension to support the first metatarsal head of the wearer's foot;

FIG. 6, a rear elevational view of the bi-planar rear post taken on the line 6-6 of FIG. 1 showing a rigid plastic upper plate and a resilient foam wedge;

FIG. 7, a perspective view of the first embodiment user-customizable insole showing respective mid-portions of the upper insole and the base insole affixed together such that the heel and forefoot portions of the upper insole may be elevated to install and remove the insole adjustment devices;

FIG. 8, an exploded perspective view of the first embodiment user-customizable insole as used with a second version insole customizing kit which includes a full length medial wedge and a split heel wedge;

FIG. 9, an exploded perspective view of the medial wedge which slips over the first metatarsal support extension and extends longitudinally along a lower surface of the base insole;

FIG. 10, a rear elevational view of the split heel wedge taken on the line 10-10 of FIG. 8;

FIG. 11, an exploded perspective view of a second embodiment user-customizable insole which includes a second version upper insole of two-thirds length and a second version base insole of two-thirds length having a heel portion with a smaller, oval-shaped flat bottom with an attached horseshoe-shaped heel support;

FIG. 12, a front elevational view of the heel support taken on the line 12-12 of FIG. 11;

FIG. 13, a top plan view of a third version base insole of two-thirds length having a metatarsal arch support which is perforated with a plurality of round holes to provide increased flexing thereof;

FIG. 14, a top plan view of a fourth version base insole of two-thirds length having a metatarsal arch support and a mid-portion which are perforated with a plurality of round holes to provide increased flexing thereof;

FIG. 15, a top plan view of a fifth version base insole of two-thirds length having a metatarsal support, a mid-portion, and a lateral cuboid support which are perforated with a plurality of slots to provide increased flexing thereof;

FIG. 16, an exploded perspective view of a third embodiment user-customizable insole as used with a third version insole customizing kit, the insole including a third version upper insole of full length and the first version base insole, the third version insole customizing kit including a forefoot medial wedge and a medial half heel wedge;

FIG. 17, an exploded perspective view of the forefoot medial wedge;

FIG. 18, a front elevational view of the medial half heel wedge taken on the line 18-18 of FIG. 16;

FIG. 19, an exploded perspective view of a fourth embodiment user-customizable insole as used with a contoured heel pad, the insole including the third version upper insole and a sixth version base insole of two-thirds length comprising the first version base insole with the first metatarsal head extension tab removed;

FIG. 20, an exploded perspective view of a fifth embodiment user-customizable insole as used with a lateral half heel wedge, the insole including a fourth version upper insole of full length and the first version base insole;

FIG. 21, an exploded perspective of a sixth embodiment user-customizable insole which includes a fifth version upper insole of full length and the first version base insole;

FIG. 22, an exploded perspective view of a seventh embodiment user-customizable insole as used with a fourth version insole customizing kit, the insole including the third version upper insole and the first version base insole, the fourth version insole customizing kit including a metatarsal arch support pad and an underfoot support pad;

FIG. 23, an exploded perspective view of an eighth embodiment user-customizable insole which includes the fifth version upper insole and a seventh version base insole of two-thirds length having a slotted forefoot portion;

FIG. 24, an exploded perspective view of a ninth embodiment user-customizable insole which includes the fourth version upper insole and an eighth version base insole of two-thirds length having an integrally molded heel support having a metatarsal support and a longitudinal plantar fascia ligament support which are perforated with a plurality of round holes to provide increased flexing thereof;

FIG. 25, an exploded perspective view of a tenth embodiment user-customizable insole which includes the fourth version upper insole and a ninth version base insole of two-thirds length having a ribbed lower surface;

FIG. 26, an exploded perspective view of an eleventh embodiment user-customizable insole as used with a gel heel pad, the insole including the second version upper insole and a tenth version base insole of full length;

FIG. 27, an exploded perspective view of a twelfth embodiment user-customizable insole as used with a fifth version insole customizing kit, the insole including the second version upper insole and an eleventh version base insole of two-thirds length having an attached heel support, a slotted forefoot portion, and a longitudinal plantar facia ligament support with a plurality of round holes to provide increased flexing thereof, the fifth version insole customizing kit including the lateral half-heel wedge, a metatarsal arch support pad, and a pivot pad;

FIG. 28, a top plan view of the eleventh version base insole showing the slotted forefoot portion and the perforated longitudinal plantar facia ligament support;

FIG. 29, an exploded perspective view of a thirteenth embodiment user-customizable insole which includes the second version upper insole and a twelfth version base insole of two-thirds length having an attached pin-located heel support;

FIG. 30, a top plan view of the heel support taken on the line 30-30 of FIG. 29 showing a plurality of locating pins extending from a concave upper surface of the heel support;

FIG. 31, an exploded perspective view of a fourteenth embodiment user-customizable insole as used with a sixth version insole customizing kit, the insole including a seventh version upper insole of two-thirds length and the eleventh version base insole having an attached heel support, the sixth version insole customizing kit including a second version first metatarsal head boot, the metatarsal arch support pad, and the pivot pad;

FIG. 32, an exploded perspective view of a fifteenth embodiment user-customizable insole which includes a unitary insole of full length and an adjustable toe grip kit;

FIG. 33, an exploded perspective view of the fifth embodiment user-customizable insole as used with a seventh version insole customizing kit, the insole including the fourth version upper insole and the first version base insole, the seventh version insole customizing kit including a metatarsal arch spring support and a lateral cuboid spring support;

FIG. 34, an exploded perspective view of a sixteenth embodiment user-customizable insole, as used with an eighth version insole customizing kit, the insole including the fourth version upper insole and a thirteenth version base insole of two-thirds length, the eighth version insole customizing kit including a metatarsal arch compression spring, a lateral cuboid compression spring, and a medial longitudinal arch support pad;

FIG. 35, an exploded perspective view of a seventeenth embodiment user-customizable insole as used with the eighth version insole customizing kit, the insole including an eighth version upper insole off till length, a first version partial base insole with an optional first version partial base support which is perforated;

FIG. 36, a lateral vertical sectional view of the first version partial base insole with the first version partial base support assembled thereto taken on the line 36-36 of FIG. 35, showing how a metatarsal arch spring support of the eighth version insole customizing kit fits therebetween; and

FIG. 37, a perspective view of a second version partial base insole which is slotted with an optional second partial base support which is slotted assembled thereto.

### DETAILED DESCRIPTION OF THE ILLUSTRATED EMBODIMENTS

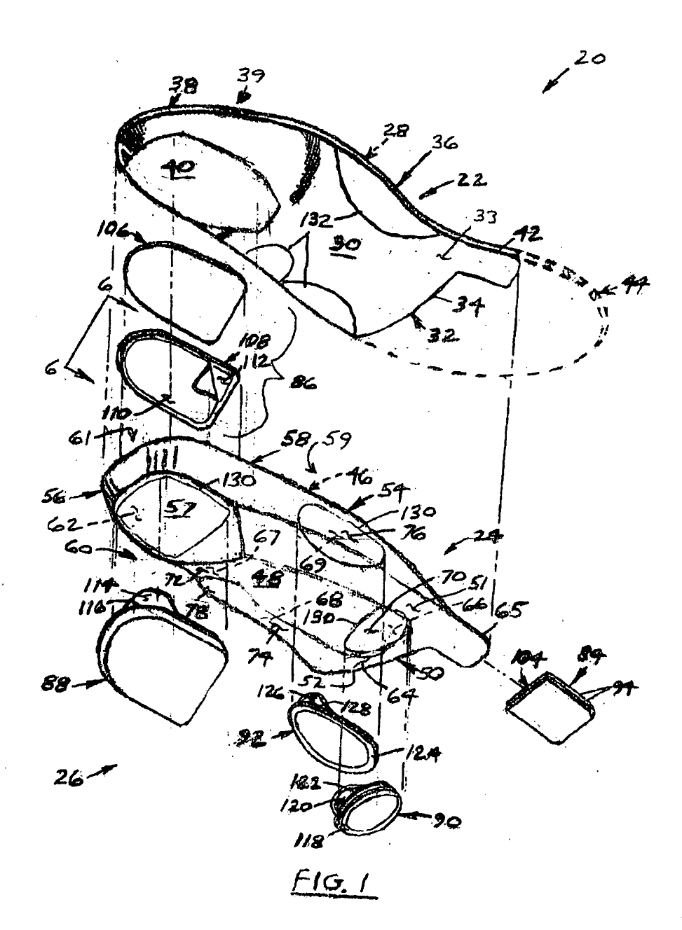

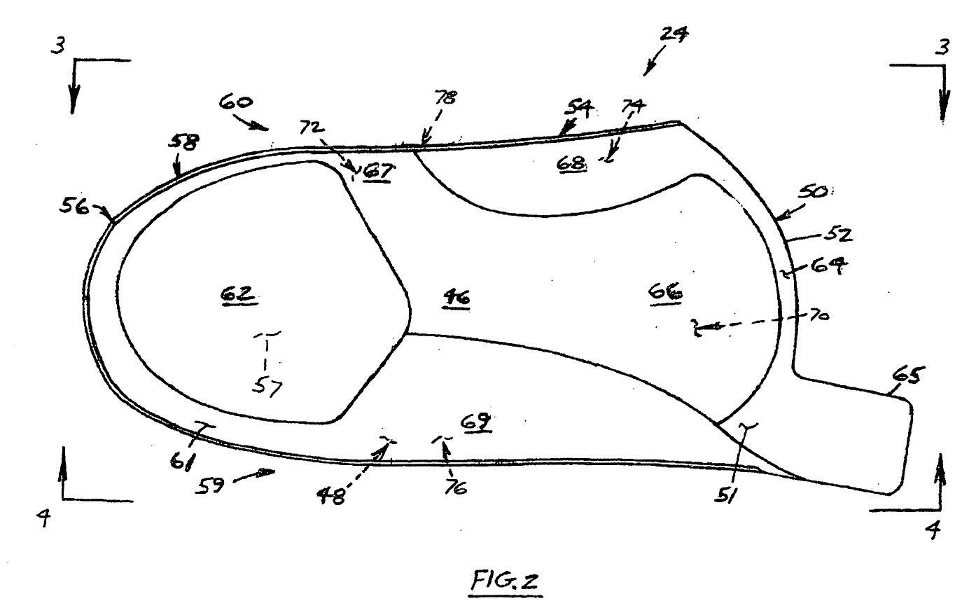

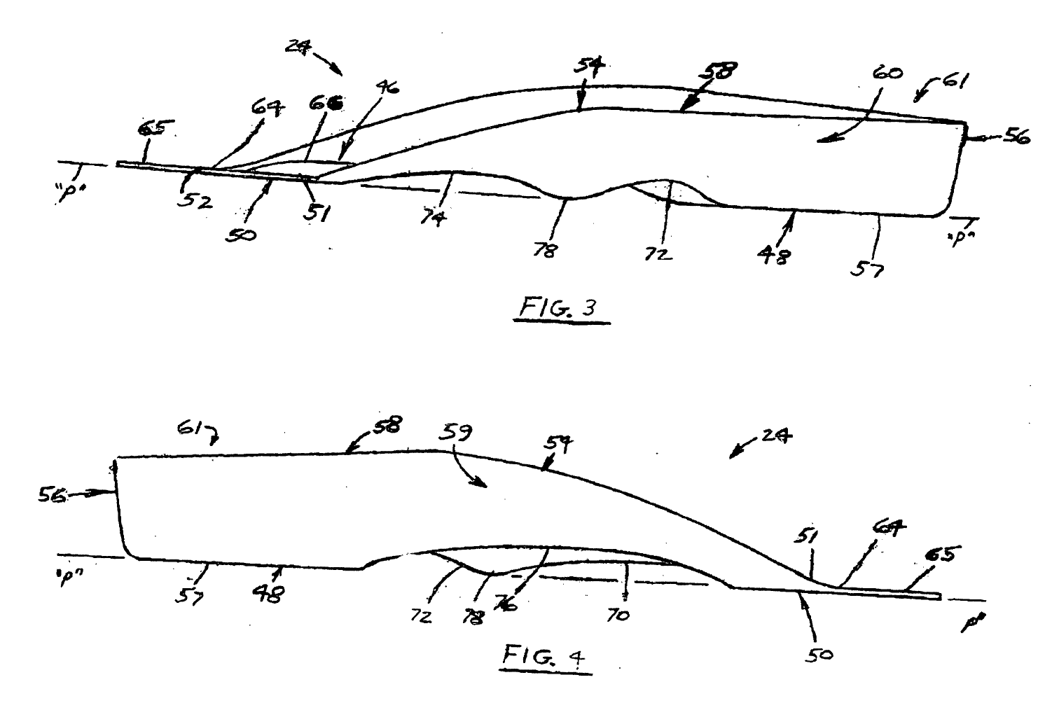

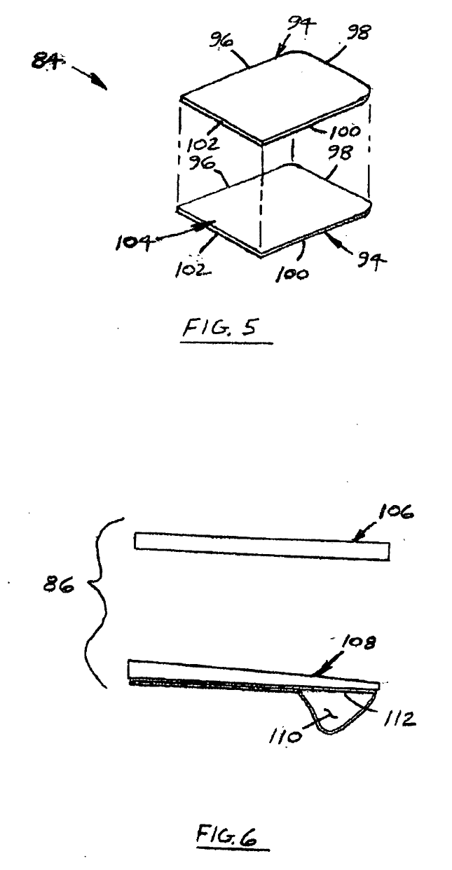

As shown in FIGS. 1-7, a first embodiment user-customizable insole in accordance with the present invention is designated generally at 20. The insole 20, as well as those which follow, are for placement onto the inside bottom of all types of conventional and specially-made footwear under the wearer's foot in place of the standard removable insole made at the factory. The footwear, for example, may be in the form of athletic shoes, dress shoes, casual shoes, sandals, and the like (hereinafter these and all others referred to as footwear). The insoles may be sized and shaped in various lengths, widths, and thicknesses with the desired built-in orthopedic contour to accommodate users having differing foot sizes and widths, arch sizes, and the like. The insoles are shown in a left foot configuration which may be mirrored to produce a right foot configuration.

The insole 20 includes a first version upper insole 22 of two-thirds length and a first version base insole 24 of two-thirds length, and is shown as used with a first version insole customizing kit 26. The insole 20 is especially preferred for dress shoes which typically have only minimal toe space.

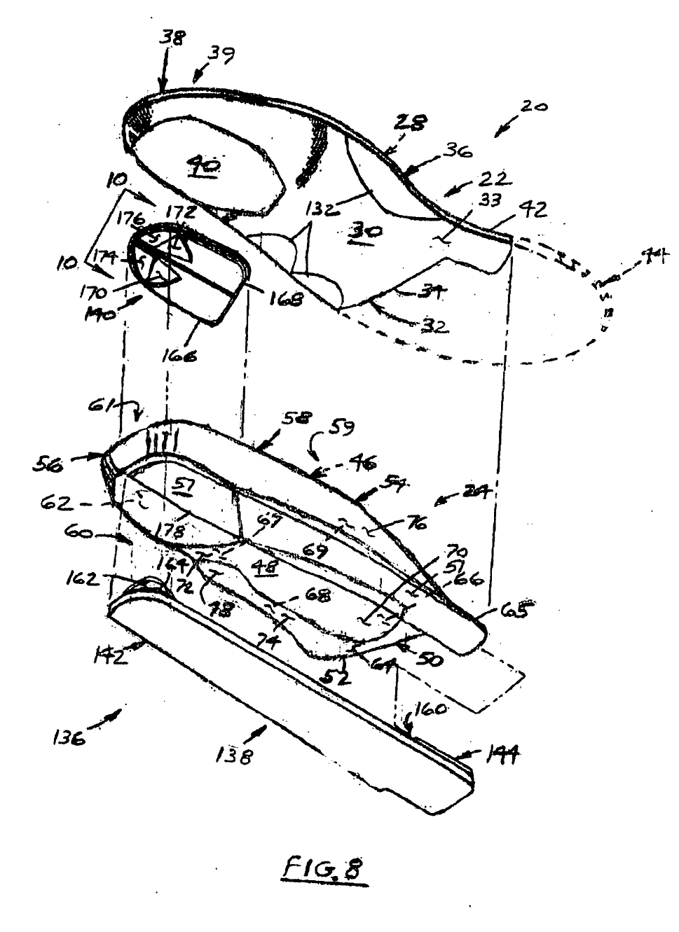

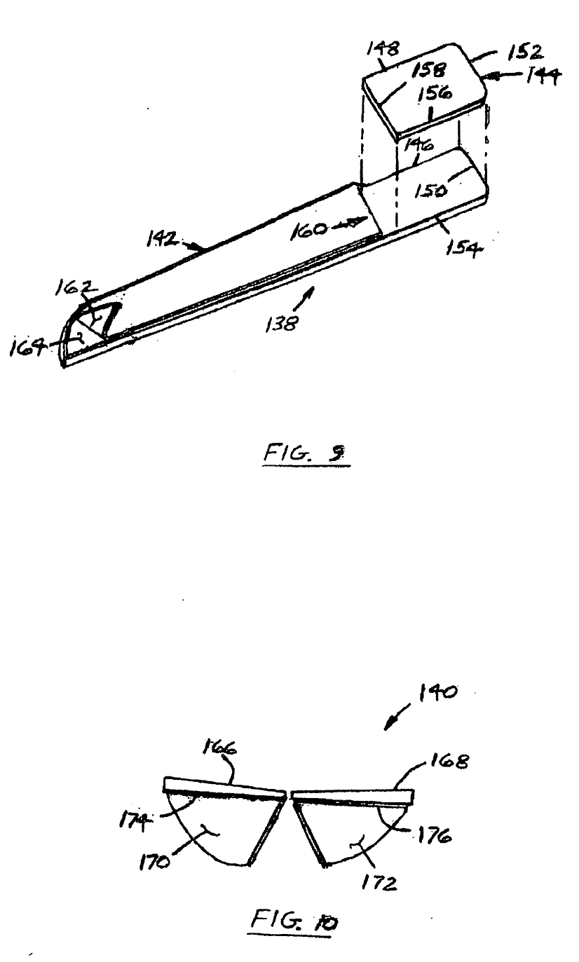

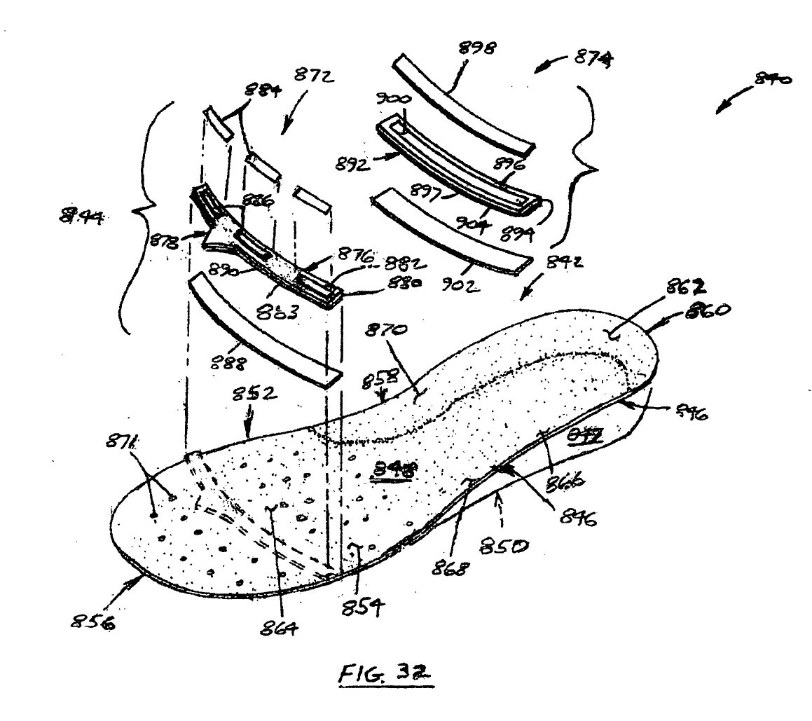

The upper insole 22 is of a flexible homogenous or monolithic construction made of a single resilient cushioning material, such as ethylene-vinyl acetate polymer (EVA), though other similar cushioning materials including open and closed cell foams may be used. Other upper insoles of various constructions follow such as the flexible monolithic construction (FIGS. 8, 11, 20-25, 27, and 29), a flexible sandwiched construction (FIGS. 16, 19, and 26), a cloth construction (FIG. 31), and a semi-rigid sandwiched construction (FIG. 32).

The upper insole 22 is shaped like the outline of the sole or plantar surface of the foot, having an upper surface 28 adapted to engage the plantar surface of the wearer's foot, and a lower surface 30 adapted to fit to the base insole 24. The upper insole 22 may be a substantially flat sheet which forms to the contours of the base insole 24 or may be molded with contours to match the contours of the plantar surface of the foot and of the base insole 24. The upper insole 22 includes a forefoot portion 32 positionable underneath the metatarsal bones of the wearer's foot beginning at a frontal area 33 of the upper insole 22 comprising a forward edge 34. A mid-portion 36 of the upper insole 22 interconnects the forefoot portion 32 to a heel portion 38. The heel portion 38 is positionable under the calcaneus bone of the wearer's foot and has a concave heel cup 39 to match the contour of the heel of the wearer's foot. A flat bottom 40 opposite heel cup 39 interfaces with the base insole 24 and facilitates placement of intrinsic heel adjustment devices into the base insole 24 as described subsequently. A first metatarsal head extension 42 extends forwardly as part of the frontal area 33 of the upper insole 22 to support the first metatarsal head of the wearer's foot. The first metatarsal head extension 42, as well as those which follow, may be trimmed or cut off as desired to customize fit to the particular wearer's foot. The upper insole 22 and base insole 24, as well as those which follow, may optionally be perforated to facilitate heat dissipation therethrough. The length of the upper insole 22 is optionally of full length, including a forward portion 44, as shown by the dotted lines which extends from the rear of the calcaneus bone completely to the ends of the toes or phalanges of the foot.

The base insole 24, as well as those which follow, are semi-rigid yet resiliently flexible, providing support to the upper insole and the wearer's foot. The base insoles are shaped to accept the upper insoles and to fit into the footwear. The base insoles are made so as to be resilient and moderately deflectable, being made of an elastomeric, semi-rigid plastic material which is heat formed and cut from sheet plastic material or injection molded from plastic, preferably polypropylene, though others such as polyethylene, polyvinyl chloride, and polyurethane may be used. The base insoles are preferably relatively thin and of a generally uniform thickness with an average thickness of between about 0.032 and 0.096 inch so as to allow controlled flexing to support the plantar surface of the foot yet absorb shock loads. The base insole 24 includes an upper surface 46 which is contoured to provide support to the plantar surface of the wearer's foot and a lower surface 48 adapted to engage the inside bottom of the footwear. The base insole 24 is about two-thirds the length of the foot, extending from the heel to the forefoot of a wearer's foot behind the metatarsal heads of the foot.

The base insole 24 includes a forefoot portion 50 positionable underneath the metatarsal bones of the wearer's foot beginning at a frontal area 51 of the base insole 24 comprising a forward edge 52. A mid-portion 54 of the base insole 24 interconnects the forefoot portion 50 to a heel portion 56 positionable underneath the calcaneus bone of the wearer's foot which includes a flat bottom 57. A tapered perimeter wall 58 extends around heel portion 56 and forwardly along respective medial and lateral sides 59 and 60 of the insole 20 defining a concave heel cup 61 having a large flat bottom 62. The perimeter wall 58 is highest about the heel portion 56, tapering downwardly through the mid-portion 54, and tapering away at about the forefoot portion 50. The forefoot portion 50 includes a narrow support lip 64 disposed from the forward edge 52 rearwardly. The support lip 64, as well as those which follow, is preferably flat to strengthen and minimize cracking of the base insole 24 and the forward edge 52, as well as those which follow, is preferably feathered (i.e. gradually tapered to a fine edge), beveled, or otherwise tapered to reduce any digging in and cutting of the inside bottom of the footwear, and to avoid the feel of an edge where the base insole 24 ends and the inside bottom begins. A first metatarsal head extension 65 extends forwardly as part of the frontal area 51 of the base insole 24 to support the first metatarsal head of the wearer's foot. The first metatarsal head extension 65, as well as those which follow, is preferably relatively thin being between about 0.010 and 0.095 inch thick to prevent cracking and discomfort to the fool. The first metatarsal head extension 65, as well as those which follow, functions as a foundation that accepts slip-on insole adjustment devices of a desired thickness as needed to control pronation at toe-off during striding and while standing. The first metatarsal head extension 65 may be trimmed or cut off as desired to customize fit to the particular wearer's foot.

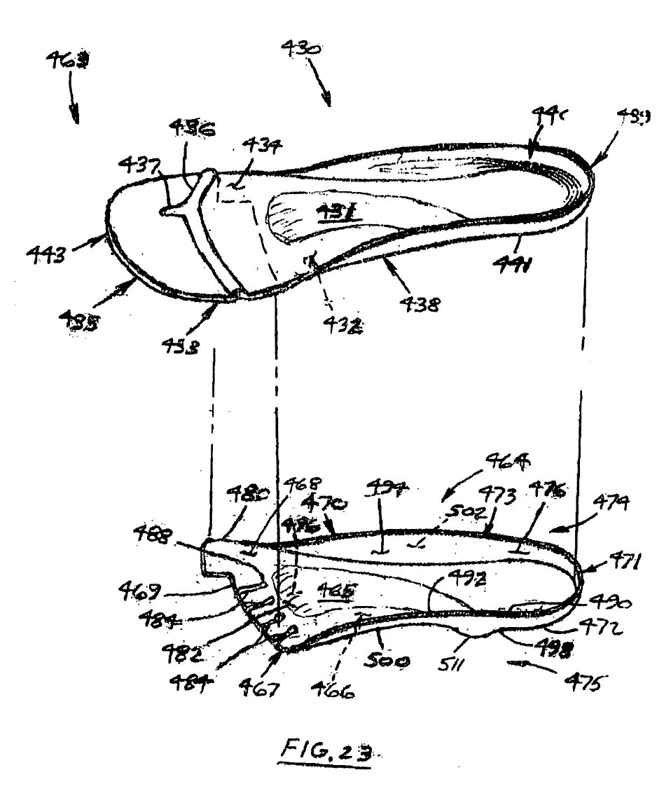

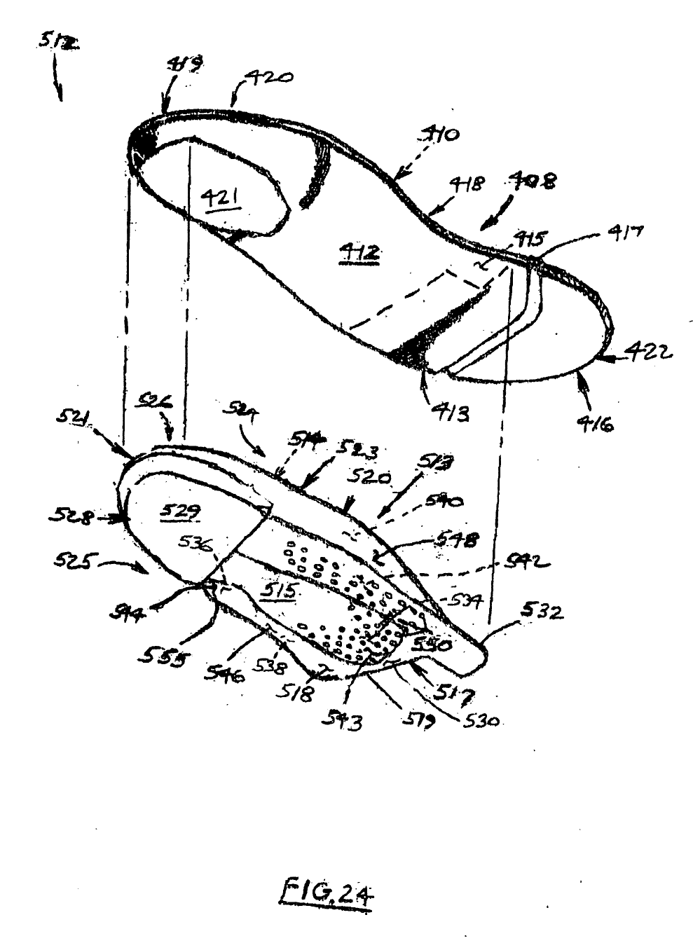

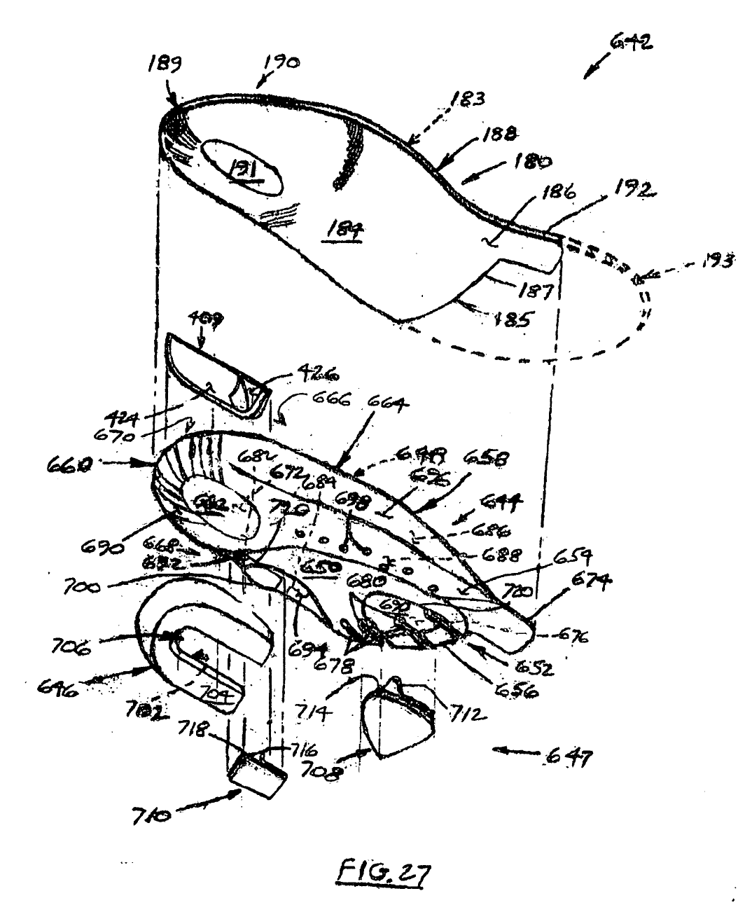

The base insole 24 has a built-in orthopedic contour, including a plurality of integral foot supports. An upwardly curved metatarsal arch support 66 is formed in base insole 24 to support of the metatarsal bones of the foot. The metatarsal arch support 66 begins at the forward edge 52 of the frontal area 51. An upwardly curved lateral cuboid support 67 is formed in base insole 24 to support the cuboid bone of the foot. An upwardly curved lateral arch support 68 is formed in base insole 24 to support the lateral arch curve of the foot. An upwardly curved medial longitudinal arch support 69 is formed in base insole 24 to support the medial longitudinal arch curve of the foot. The medial longitudinal arch support 69 provides support for the calcaneus or heel bone of the foot in relation to the plantar fascia to help prevent the calcaneus bone from rotating to a more horizontal orientation as happens in the abnormal foot condition called fallen arches. The underside of the foot supports 66, 67, 68, and 69 comprise corresponding upwardly curved elevation curves 70, 72, 74, and 76 which are disposed above the inside bottom of the footwear. Each elevation curve 70, 72, 74, and 76 defines a corresponding space (not shown) with the inside bottom of the footwear such that the foot supports 66, 67, 68, and 69 may flex downwardly under a predetermined foot pressure providing a suspension effect to reduce shock and provide selected support to the wearer's foot and provide greater comfort during walking, jogging, or running. The predetermined foot pressure of each foot support 66, 67, 68, and 69 is controlled by using at least one base insole strength modifying feature, such as adding one or more perforations which are round, slots, or other shape as described for FIGS. 13-15, 24, 27, 28, and 31, adding open-ended slots as described in FIGS. 23, 27, 28, and 31, thinning the material of the base insole 24 at the foot support 66, 67, 68, and 69, adding external material to selectively strengthen the slots such as Kevlar or other adhesive tape (not shown) to the perforations and/or open-ended slots, and regional changes to mechanical properties of material of the base insole such as selectively increasing the thickness of the base insole or gusseting. The base insole 24 includes a downwardly extending lateral pivot 78 having a lowermost portion which is at a position relative to a bottom plane “P” of the base insole 24 preferably either slightly above or even with the bottom plane “P”. The lateral pivot 78 is disposed below the protuberance base of the fifth metatarsal shaft of the foot to in guiding the foot medially to the big toe for toe-off during striding and is used to adjust supination of the foot during striding, as shown in FIGS. 27 and 31.

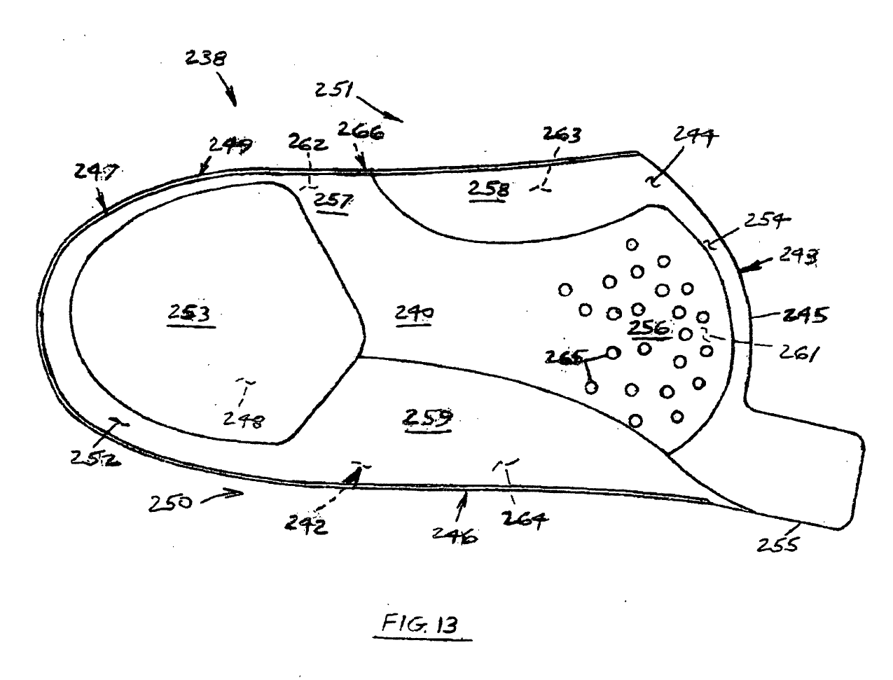

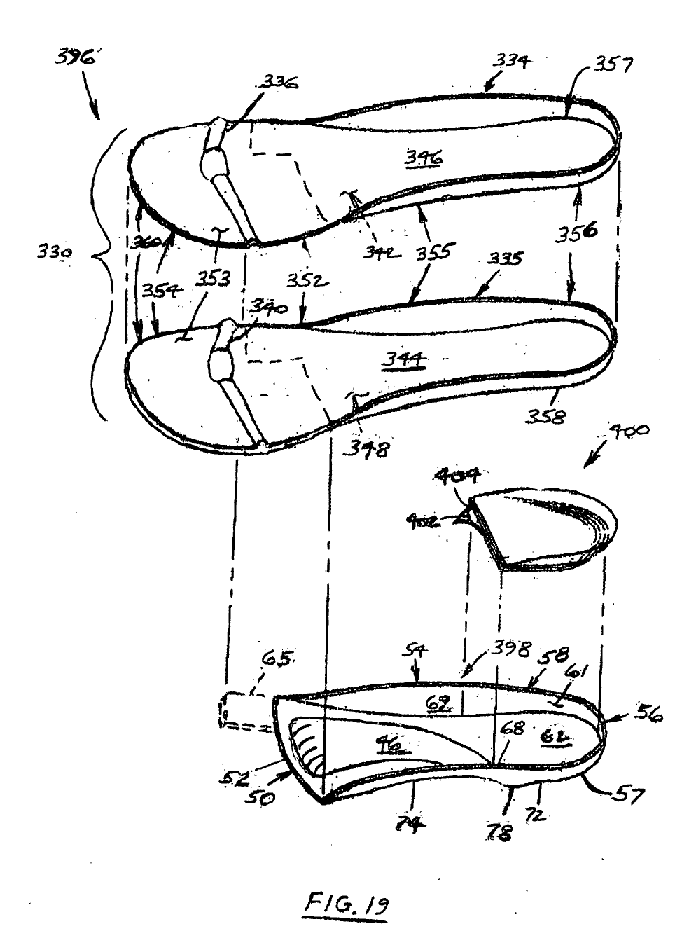

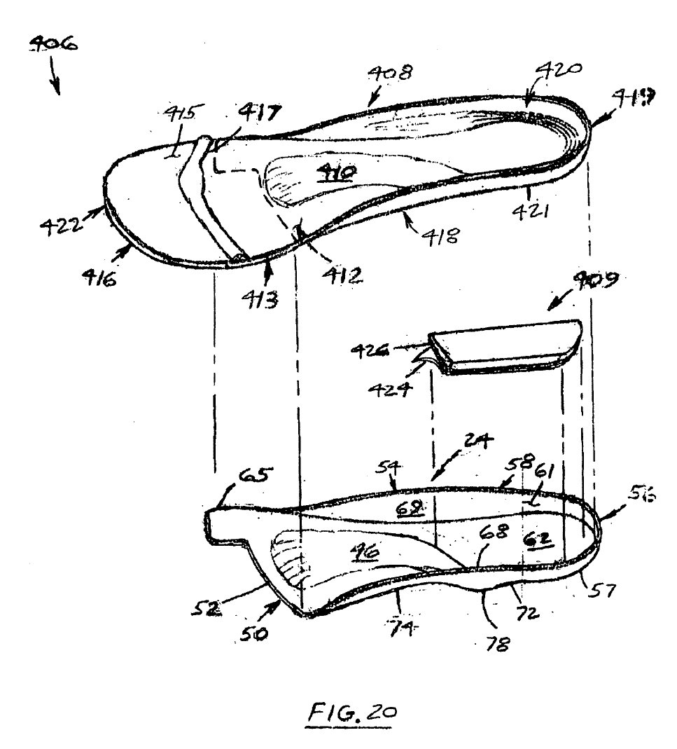

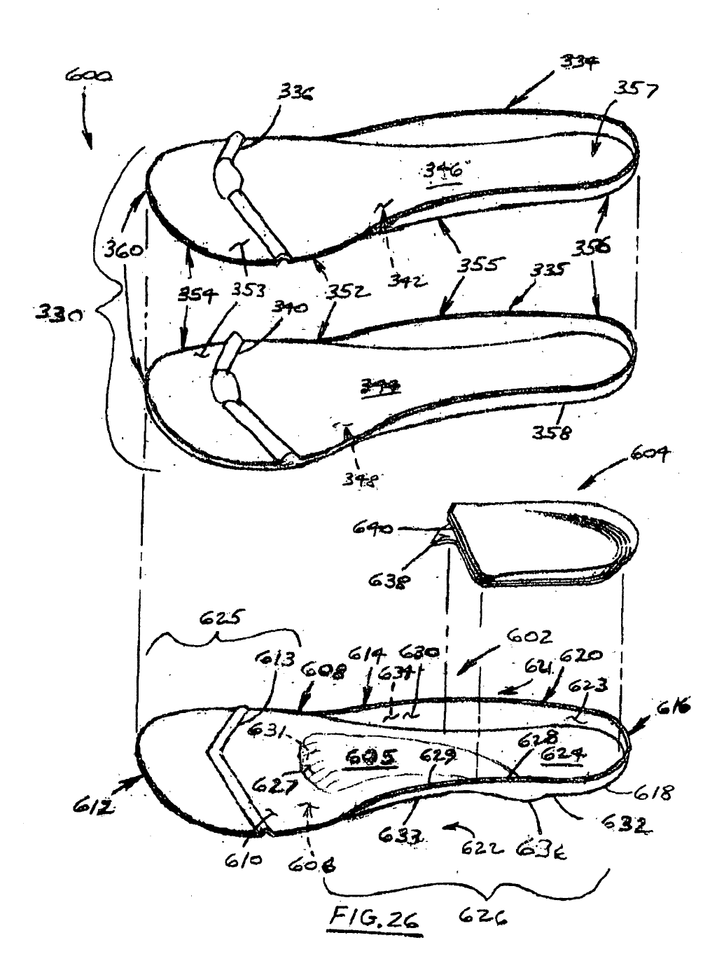

The heel cup 61, as well as those which follow, is of a sufficient depth and the large flat bottom 62 provides a stable platform for intrinsic adjustment devices such as full and half wedges used to correct rear foot pronation and supination (FIGS. 1, 6, 7, 8, 10, 16, 18, 20, and 27), cushioning heel pads (FIGS. 19 and 26), and other intrinsic adjustment devices for support of the calcaneus heel bone of the talus area of the wearer's foot. Extrinsic adjustment devices such as heel lifts used for short leg correction, shock absorbing materials, longitudinal medial wedges, and underfoot pads may also be placed below the flat bottom 57 for extra support and comfort (FIGS. 1, 7-9, and 22).

The first version insole customizing kit 26 includes intrinsic and extrinsic insole adjustment devices adapted for use with insole 20 and those which follow for adjusting positioning of the wearer's foot within the foot wear. Intrinsic insole adjustment devices are disposed between the base insole 24 and the upper insole 22. Extrinsic insole adjustment devices are disposed on the base insole 24 and/or the upper insole 22 other than therebetween. The first version insole customizing kit 26 includes a first version first metatarsal head extension boot 84, a bi-planar rear post 86, a heel lift 88, a metatarsal arch support pad 90, and a longitudinal arch support pad 92. As shown in FIGS. 1 and 5, the boot 84 preferably comprises a pair of thin foam sheets 94 which are affixed together at respective edges 96, 98, and 100. Respective free edges 102 remain unaffixed so as to form a pocket 104 for receiving the first metatarsal head extension 65. The boot 84 slips onto the first metatarsal head extension 65 and may be made in different thicknesses as required to control pronation at toe off during striding and while standing. As shown in FIGS. 1 and 6, the rear post 86 includes a hard plastic upper plate 106 and a foam wedge 108 that are adhesively bonded together. A peel-off backing paper or sheet 110 exposes an adhesive layer 112 to secure rear post 86 within the heel cup 61. The rear post 86 corrects the initial heel and foot position and provides increased heel stability and alignment during striding. The heel lift 88 is made of a resilient material, preferably a sheet of foam, preferably avisco-elastic foam, with a peel-off backing sheet 114 exposes an adhesive layer 116 to secure heel lift 88 to below the bottom 57. The heel lift 88 corrects for differences in leg length, and provides increased shock absorption in the heel of the footwear. The metatarsal arch support pad 90 is made of a resilient material, preferably a circular sheet of foam, preferably a visco-elastic foam, having a tapered lower peripheral edge 118, with a peel-off backing sheet 120 exposes an adhesive layer 122 to secure metatarsal arch support pad 90 to the elevation curve 70 below the metatarsal arch support 66. The metatarsal arch support pad 90 provides increased support for the metatarsal bones of the foot. The metatarsal arch support pad 90 may be made of a more solid material if more rigid support is desired. The longitudinal arch support pad 92 is made of a resilient material, preferably a rounded sheet of foam, preferably a visco-elastic foam, having a tapered peripheral edge 124, with a peel-off backing sheet 126 exposes an adhesive layer 128 to secure longitudinal arch support pad 92 to the elevation curve 76 below the medial longitudinal arch support 69. The longitudinal arch support pad 92 provides increased support for the medial longitudinal arch of the foot. The longitudinal arch support pad 92 may be made of a more solid material if more rigid support is desired. The adjustment devices including the rear post 86, the heel lift 88, the metatarsal arch support pad 90, and the longitudinal arch support pad 92, as well as the other adjustment devices described below, are easily removed when less support is desired and may alternatively be affixed to the base insole 24 using strips of conventional double-sided adhesive tape (not shown).

The base insole 24, as well as those which follow, preferably includes positioning indicia 130 etched or printed thereon providing placement instructions and which marks the locations for correct placement of the adjustment devices thereon, including the rear post 86, the heel lift 88, the metatarsal arch support pad 90, and the longitudinal arch support pad 92. The upper insole 22, as well as those which follow, may also include positioning indicia similar to base insole 24 such as indicia 132 etched or printed thereon outlining the plantar contours of the foot to aid in placement of the insole adjustment devices.

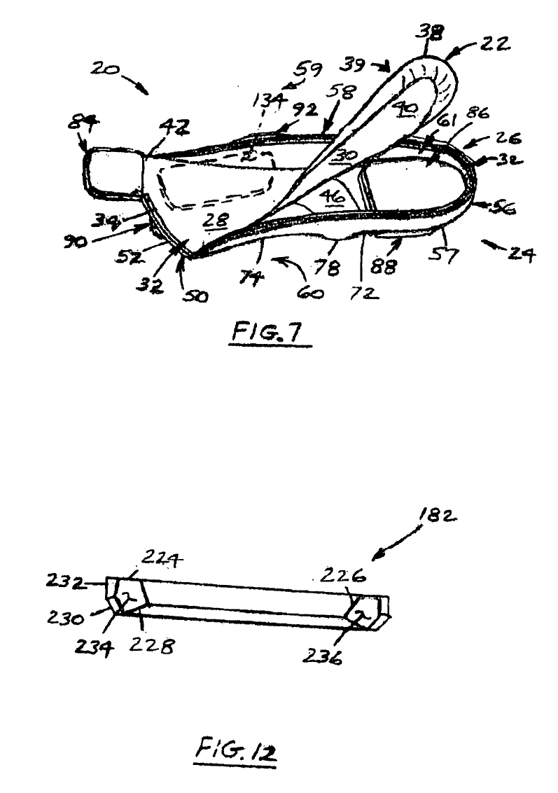

As shown in FIG. 7, as well as for those insoles which follow, the mid-portion 36 of the upper insole 22 is preferably affixed to the mid-portion 54 of the base insole 24 using an adhesive layer 134 on the upper surface 46 of base insole 24. Alternatively, the upper insoles may not be affixed at all to the base insoles so as to be fully removable therefrom. The forefoot portion 32 and the heel portion 38 of the upper insole 22 remain unaffixed to base insole 24 such that the forefoot portion 32 and the heel portion 38 may be raised from the base insole 24 to add and remove the adjustment devices such as the boot 84 and the rear post 86. Therefore a wearer may easily remove the insole 20 from the footwear, lift the forefoot portion 32 to replace forefoot and toe adjustment devices, lift the heel portion 38 to replace the rear post 86 with other heel adjustment devices, and replace the insole 20 back into the footwear. The upper insole 22 may alternatively be attached to the base insole 24 using double-sided adhesive tape (not shown) rather than the adhesive layer 134. The wearer may also use the insole 20 by laying the upper insole 22 onto the base insole 24 without affixing together and placing into the footwear. Additional material may be inserted between the first metatarsal head extension 42 and the first metatarsal head extension 65 instead of or in addition to the boot 84 for increased first metatarsal support.

The insole 20, as well as those which follow are, are used by placing in the footwear with the lower surface 48 of the base insole 24 contacting the inside bottom of the footwear and the upper surface 28 of the upper insole 22 contacting the plantar surface of the foot. When the insole 20 is worn, the weight of the shoe wearer on the foot forces the upper insole 22 downwardly on the base insole 24, with the contours of the upper surface 46 of base insole 24 supporting the upper insole 22 and thus the plantar surface of the foot. The insole 20 thus aids the foot by limiting the degree to which the metatarsal arch, the cuboid bone, the lateral arch, and the longitudinal arch may flatten when weight-bearing, such as during the gait cycle of a walking or running. The configuration and resiliency of the insole 20 is such that the base insole 24 can deflect downwardly to a moderate extent upon the application of pressure from the foot and upon release of the weight on the foot will again spring back to its original position.

The insole 20 may be adjusted by a wearer for the abnormally functioning foot by adjusting the insole 20 using the first version insole customizing kit 26 to fine tune the insole 20 as their particular needs dictate. For example, if the wearer needs the pronation or supination adjusted, the appropriate boot 84 and the rear post 86 may be utilized. If more arch support is needed, the metatarsal arch support pad 90 and/or the longitudinal arch support pad 92 may be utilized. The rear post 86 and the heel lift 88 may be used to correct for a short leg. The insole 20 benefits the over-pronated foot by providing more medial stabilizing support in the heel and ankle area. The insole 20 benefits the supinated foot by being contoured to the individual arches helping to distribute body weight more evenly. The heel lift 88 also absorbs shocks during striding to protect the heel from excessive impact forces. Even stable feet are benefitted by allowing the wearer to give a custom fit to the footwear, which is particularly desirable where a precise fit is important such as in athletic footwear.

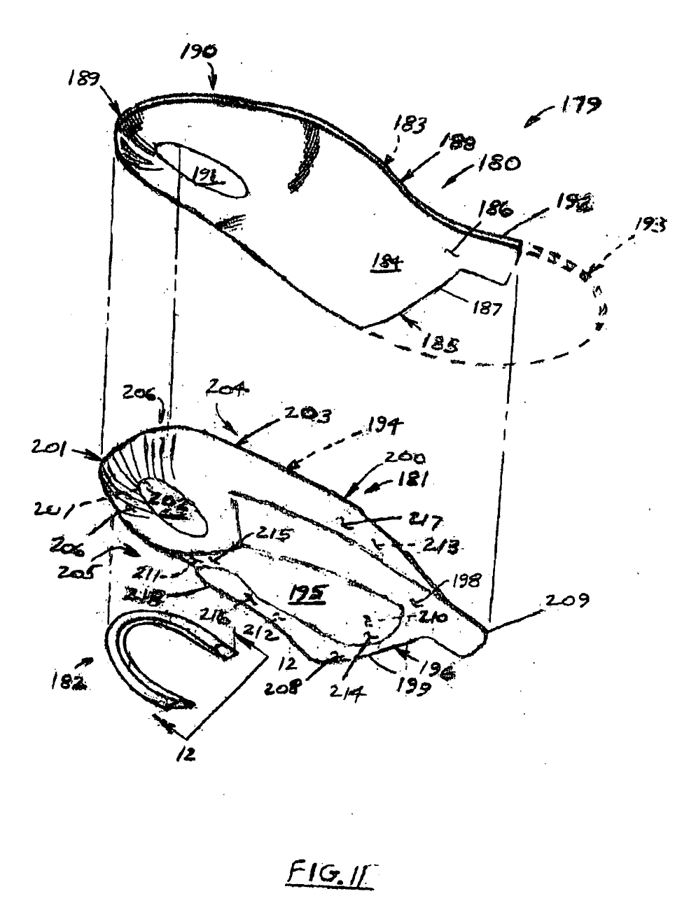

Referring to FIGS. 8-10, therein is shown the insole 20 as used with a second version insole customizing kit 136 which features both intrinsic and extrinsic insole adjustment devices. The second version insole customizing kit 136 includes a full length medial wedge 138, and a split heel wedge 140. As shown in FIG. 9, the medial wedge 138 comprises respective elongate and short foam sheets 142 and 144 which are affixed together at respective edges 146 and 148, 150 and 152, and 154 and 156. A free edge 158 remains unaffixed so as to form a first metatarsal head extension receiving pocket 160. The medial wedge 138 slips onto the metatarsal head extension 65 to provide correct comfort and support for the first metatarsal head of the foot. A peel-off backing sheet 162 exposes an adhesive layer 164 to further secure medial wedge 138 to base insole 24. The medial wedge 138 allows correction of pronation or supination particularly during toe-off during striding. The split heel wedge 140 includes a pair of foam wedges 166 and 168. A pair of peel-off backing sheets 170 and 172 expose respective adhesive layers 174 and 176 to secure wedges 166 and 168 to the flat bottom 62 inside heel cup 61. A single wedge 166 or 168 may also be used to provide extra support on the lateral or medial side of the wearer's heel. The base insole 24 preferably includes an alternate positioning indicia 178 etched or printed thereon providing placement instructions and which marks the locations to facilitate placement of the adjustment devices thereon including the medial wedge 138 and the split heel wedge 140.

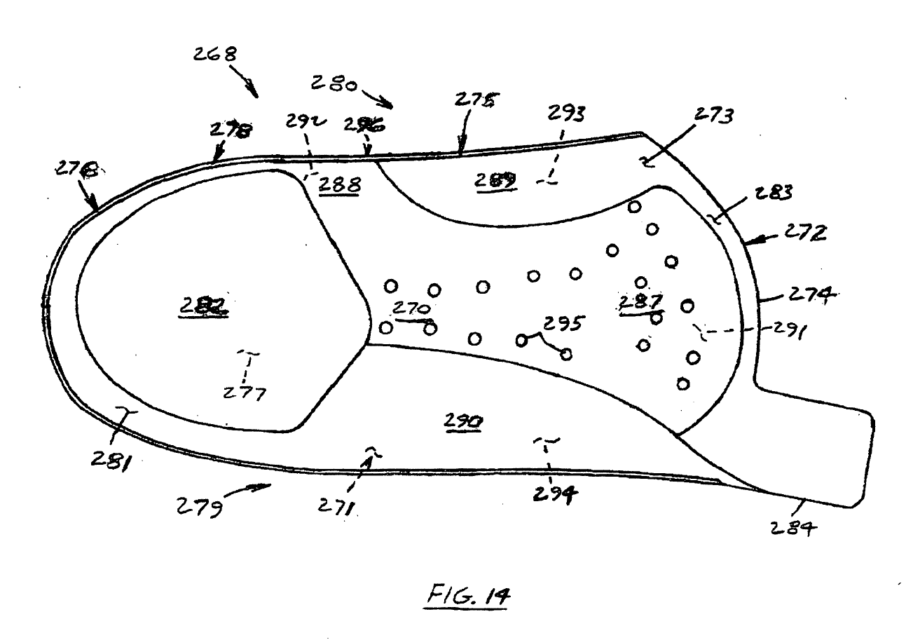

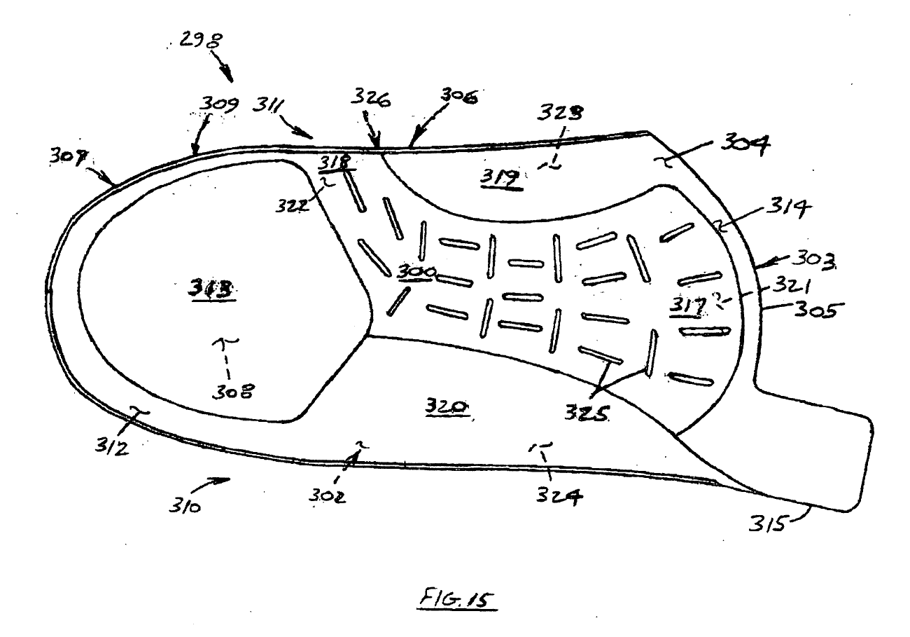

Referring to FIGS. 11 and 12, therein is shown a second embodiment user-customizable insole 179. The insole 179 includes a second version upper insole 180 of two-thirds length, and a second version base insole 181 of two-thirds length with a separately molded heel support 182.

The upper insole 180 is relatively soft providing cushioning for the foot and shaped to fit into the footwear. The upper insole 180 is of the flexible monolithic construction made of a resilient cushioning material, such as ethylene-vinyl acetate polymer (EVA), though other similar cushioning open or closed cell foams may be used. The upper insole 180 is shaped like the outline of the sole or plantar surface of the foot, having an upper surface 183 adapted to engage the plantar surface of the wearer's foot, and a lower surface 184 adapted to fit to the base insole 181. The upper insole 180 may be a substantially flat sheet which forms to the contours of the base insole 181 or may be molded with contours to match the contours of the plantar surface of the foot and of the base insole 181. The upper insole 180 includes a forefoot portion 185 positionable underneath the metatarsal bones of the wearer's foot beginning at a frontal area 186 of the upper insole 180 comprising a forward edge 187. A mid-portion 188 of the upper insole 180 interconnects the forefoot portion 185 to a heel portion 189. The heel portion 189 is positionable under the calcaneus bone of the wearer's foot has a concave heel cup 190 to match the contour of the heel of the wearer's foot and a flat bottom 191 opposite heel cup 190 to interface with the base insole 181 and adapted to facilitate placement of intrinsic heel adjustment devices into the base insole 181. A first metatarsal head extension 192 extends forwardly as part of the frontal area 186 of the upper insole 180 to support the first metatarsal head of the wearer's foot. The length of the upper insole 180 is optionally of full length including a forward portion 193, as shown by the dotted lines, to extend from the heel to the toes of the foot.

The base insole 181 includes an upper surface 194 which is contoured to provide support to the plantar surface of the wearer's foot and a lower surface 195 adapted to engage the inside bottom of the footwear. The base insole 181 is about two-thirds the length of the foot, extending from the heel to the forefoot of a wearer's foot behind the metatarsal heads of the foot. A forefoot portion 196 is positionable underneath the metatarsal bones of the wearer's foot beginning at a frontal area 193 of the base insole 181 comprising a forward edge 199. A mid-portion 200 of the base insole 181 interconnects the forefoot portion 196 to a heel portion 201 positionable underneath the calcaneus bone of the wearer's foot and has a large flat bottom 202. A tapered perimeter wall 203 extends around heel portion 201 and forwardly along respective medial and lateral sides 204 and 205 of the insole 179 defining a concave heel cup 206 with an oval flat bottom 207. The perimeter wall 203 is highest about the heel portion 201, tapering downwardly through the mid-portion 200, and tapering away at about the forefoot portion 196. The forefoot portion 196 includes a narrow flat support lip 208 disposed from the forward edge 199 rearwardly. A first metatarsal head extension 209 extends forwardly as part of the frontal area 198 of the base insole 181 to support the first metatarsal head of the wearer's foot.

The base insole 181 has a built-in orthopedic contour, including a plurality of integral foot supports. An upwardly curved metatarsal arch support 210 is formed in base insole 181 to support of the metatarsal bones of the foot. The metatarsal arch support 210 begins at the forward edge 199 of the frontal area 198. An upwardly curved lateral cuboid support 211 is formed in base insole 181 to support the cuboid bone of the foot. An upwardly curved lateral arch support 212 is formed in base insole 181 to support the lateral arch curve of the foot. An upwardly curved medial longitudinal arch support 213 is formed in base insole 181 to support the medial longitudinal arch curve of the foot. The medial longitudinal arch support 213 provides support for the calcaneus bone of the foot in relation to the plantar fascia. The underside of the foot supports 210, 211, 212, and 213 comprise corresponding upwardly curved elevation curves 214, 215, 216, and 217 which are disposed above the inside bottom of the footwear. Each elevation curve 214, 215, 216, and 217 defines a corresponding space (not shown) with the inside bottom of the footwear such that the foot supports 210, 211, 212, and 213 may flex downwardly under a predetermined foot pressure providing a suspension effect to reduce shock and provide selected support to the wearer's foot, and providing greater comfort during walking, jogging, or running. The predetermined foot pressure of each foot support 210, 211, 212, and 213 is controlled by using at least one base insole strength modifying feature. The base insole 181 includes a downwardly extending lateral pivot 218 having a lowermost portion which is at a position relative to a bottom plane (not shown) of the base insole 181 preferably either slightly above or even with the bottom plane. The lateral pivot 218 assists in guiding the foot medially to the big toe for toe-off during striding and is used to adjust supination of the foot during striding.

The heel support 182 is of a horseshoe shape, being polygonal in cross-section, having an upper surface 224 adapted to be affixed to the heel portion 201 of the base insole 181, an inside surface 226, a bottom surface 228, and respective outside surfaces 230 and 232. Respective opposite end surfaces 234 and 236 are rearwardly downwardly tapered. The heel support 182 is made of a resilient material, preferably a sheet of foam, preferably a visco-elastic foam, though other such resilient material such as rubber or neoprene may be used. The upper surface 224 is affixed to the heel portion 201 of the base insole 181 such as by adhesively bonding to the lower surface 195 of the base insole 181 around the heel portion 201. The heel support 182 extends downwardly from the heel portion 201 and engages the inside bottom of the footwear to resiliently stabilize the heel of the wearer's foot within the footwear. The heel support 182 supports the heel cup 206 under the wearer's body weight. The heel support 182 also acts as a shock absorber to reduce the impact force on the heel of the wearer's foot.

Referring to FIG. 13, therein is shown a third version base insole 238 of two-thirds length. The base insole 238 includes an upper surface 240 which is contoured to provide support to the plantar surface of the wearer's foot and a lower surface 242 adapted to engage the inside bottom of the footwear. The base insole 238 is about two-thirds the length of the foot, extending from the heel to the forefoot of a wearer's foot behind the metatarsal heads of the foot. The base insole 238 includes a forefoot portion 243 positionable underneath the metatarsal bones of the wearer's foot beginning at a frontal area 244 of the base insole 238 comprising a forward edge 245. A mid-portion 246 of the base insole 238 interconnects the forefoot portion 243 to a heel portion 247 positionable underneath the calcaneus bone of the wearer's foot and having a flat bottom 248 opposite heel portion 247. A tapered perimeter wall 249 extends around heel portion 247 and forwardly along respective medial and lateral sides 250 and 251 of the base insole 238 defining a concave heel cup 252 with a large flat bottom 253. The perimeter wall 249 is highest about the heel portion 247, tapering downwardly through the mid-portion 246, and tapering away at about the forefoot portion 243. The forefoot portion 243 includes a narrow flat support lip 254 disposed from the forward edge 245 rearwardly. A first metatarsal head extension 255 extends forwardly as part of the frontal area 244 of the base insole 238 to support the first metatarsal head of the wearer's foot.

The base insole 238 has a built-in orthopedic contour, including a plurality of integral foot supports. The base insole 238 is relatively thin and of a generally uniform thickness, with an average thickness of between about 0.032 and 0.096 inch. An upwardly curved metatarsal arch support 256 is formed in base insole 238 to support of the metatarsal bones of the foot. The metatarsal arch support 256 begins at the forward edge 245 of the frontal area 244. An upwardly curved lateral cuboid support 257 is formed in base insole 238 to support the cuboid bone of the foot. An upwardly curved lateral arch support 258 is formed in base insole 238 to support the lateral arch curve of the foot. An upwardly curved medial longitudinal arch support 259 is formed in base insole 238 to support the medial longitudinal arch curve of the foot. The medial longitudinal arch support 259 provides support for the calcaneus bone of the foot in relation to the plantar fascia. The underside of the foot supports 256, 257, 258, and 259 comprise corresponding upwardly curved elevation curves 261, 262, 263, and 264 which are disposed above the inside bottom of the footwear. Each elevation curve 261, 262, 263, and 264 defines a corresponding space (not shown) with the inside bottom of the footwear such that the foot supports 256, 257, 258, and 259 may flex downwardly under a predetermined foot pressure providing a suspension effect to reduce shock and provide selected support to the wearer's foot, and providing greater comfort during walking, jogging, or running. The predetermined foot pressure of each foot support 256, 257, 258, and 259 is controlled by using at least one base insole strength modifying feature, such as adding round holes 265 through metatarsal arch support 256. The base insole 238 includes a downwardly extending lateral pivot 266 having a lowermost portion which is at a position relative to a bottom plane (not shown) of the base insole 238 preferably either slightly above or even with the bottom plane. The lateral pivot 266 assists in guiding the foot medially to the big toe for toe-off during striding and is used to adjust supination of the foot during striding.

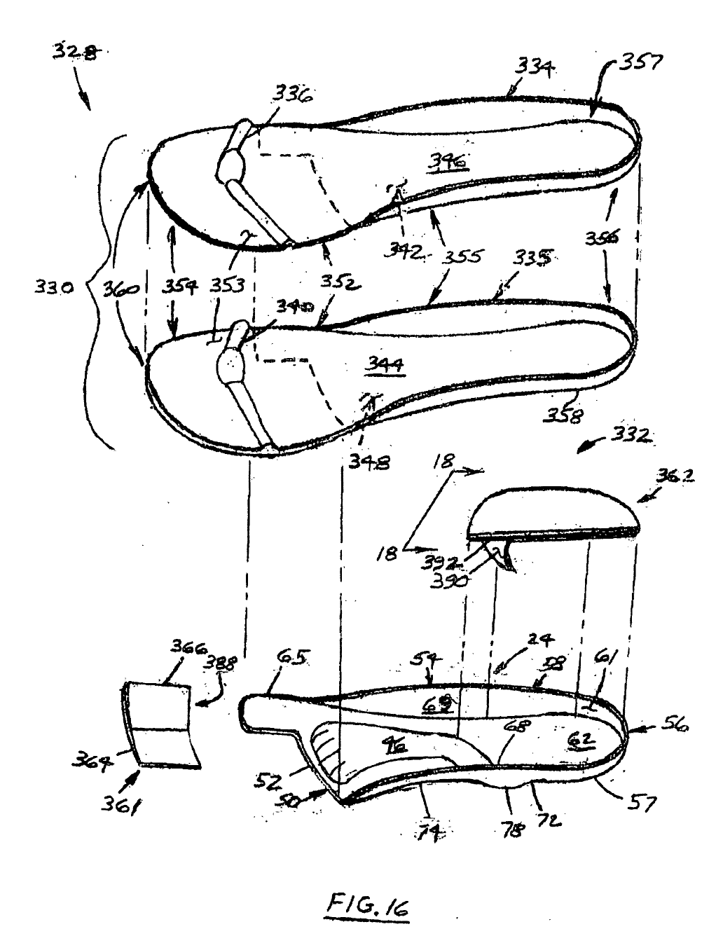

Referring to FIG. 14, therein is shown a fourth version base insole 268 of two-thirds length. The base insole 268 includes an upper surface 270 which is contoured to provide support to the plantar surface of the wearer's foot and a lower surface 271 adapted to engage the inside bottom of the footwear. The base insole 268 is about two-thirds the length of the foot, extending from the heel to the forefoot of a wearer's foot behind the metatarsal heads of the foot. The base insole 268 includes a forefoot portion 272 positionable underneath the metatarsal bones of the wearer's foot beginning at a frontal area 273 of the base insole 268 comprising a forward edge 274. A mid-portion 275 of the base insole 268 interconnects the forefoot portion 272 to a heel portion 276 positionable underneath the calcaneus bone of the wearer's foot and has a flat bottom 277. A tapered perimeter wall 278 extends around heel portion 276 and forwardly along respective medial and lateral sides 279 and 280 of the base insole 268 defining a concave heel cup 281 with a large flat bottom 282. The perimeter wall 278 is highest about the heel portion 276, tapering downwardly through the mid-portion 276, and tapering away at about the forefoot portion 272. The forefoot portion 272 includes a narrow flat support lip 283 disposed from the forward edge 274 rearwardly. A first metatarsal head extension 284 extends forwardly as part of the frontal area 273 of the base insole 268 to support the first metatarsal head of the wearer's foot.

The base insole 268 has a built-in orthopedic contour, including a plurality of integral foot supports. An upwardly curved metatarsal arch support 287 is formed in base insole 268 to support of the metatarsal bones of the foot. The metatarsal arch support 287 begins at the forward edge 274 of the forefoot portion 272. An upwardly curved lateral cuboid support 288 is formed in base insole 268 to support the cuboid bone of the foot. An upwardly curved lateral arch support 289 is formed in base insole 268 to support the lateral arch curve of the foot. An upwardly curved medial longitudinal arch support 290 is formed in base insole 268 to support the medial longitudinal arch curve of the foot. The medial longitudinal arch support 290 provides support for the calcaneus bone of the foot in relation to the plantar fascia. The underside of the foot supports 287, 288, 289, and 290 comprise corresponding upwardly curved elevation curves 291, 292, 293, and 294 which are disposed above the inside bottom of the footwear. Each elevation curve 291, 292, 293, and 294 defines a corresponding space (not shown) with the base insole 268 such that the foot supports 287, 288, 289, and 290 may flex downwardly under a predetermined foot pressure providing a suspension effect to reduce shock and provide selected support to the wearer's foot, and providing greater comfort during walking, jogging, or running. The predetermined foot pressure of each foot support 287, 288, 289, and 290 is controlled by using at least one base insole strength modifying feature, such as adding round holes 295 through metatarsal arch support 287. The base insole 268 includes a downwardly extending lateral pivot 296 having a lowermost portion which is at a position relative to a bottom plane (not shown) of the base insole 268 preferably either slightly above or even with the bottom plane. The lateral pivot 296 assists in guiding the foot medially to the big toe for toe-off during striding and is used to adjust supination of the foot during striding.

Referring to FIG. 15, therein is shown a fifth version base insole 298 of two-thirds length. The base insole 298 includes an upper surface 300 which is contoured to provide support to the plantar surface of the wearer's foot and a lower surface 302 adapted to engage the inside bottom of the footwear. The base insole 298 is about two-thirds the length of the foot, extending from the heel to the forefoot of a wearer's foot behind the metatarsal heads of the foot. The base insole 298 includes a forefoot portion 303 positionable underneath the metatarsal bones of the wearer's foot beginning at a frontal area 304 of the base insole 298 comprising a forward edge 305. A mid-portion 306 of the base insole 298 interconnects the forefoot portion 303 to a heel portion 307 positionable underneath the calcaneus bone of the wearer's foot and has a large flat bottom 308. A tapered perimeter wall 309 extends around heel portion 307 and forwardly along respective medial and lateral sides 310 and 311 of the base insole 298 defining a concave heel cup 312 with a large flat bottom 313. The perimeter wall 309 is highest about the heel portion 307, tapering downwardly through the mid-portion 306, and tapering away at about the forefoot portion 303. The forefoot portion 303 includes a narrow flat support lip 314 disposed from the forward edge 305 rearwardly. A first metatarsal head extension 315 extends forwardly as part of the frontal area 304 of the base insole 298 to support the first metatarsal head of the wearer's foot.

The base insole 298 has a built-in orthopedic contour, including a plurality of integral foot supports. An upwardly curved metatarsal arch support 317 is formed in base insole 298 to support of the metatarsal bones of the foot. The metatarsal arch support 317 begins at the forward edge 305 of the forefoot portion 303. An upwardly curved lateral cuboid support 318 is formed in base insole 298 to support the cuboid bone of the foot. An upwardly curved lateral arch support 319 is formed in base insole 298 to support the lateral arch curve of the foot. An upwardly curved medial longitudinal arch support 320 is formed in base insole 298 to support the medial longitudinal arch curve of the foot. The medial longitudinal arch support 320 provides support for the calcaneus or heel bone of the foot in relation to the plantar fascia. The underside of the foot supports 317, 318, 319, and 320 comprise corresponding upwardly curved elevation curves 321, 322, 323, and 324 which are disposed above the inside bottom of the footwear. Each elevation curve 321, 322, 323, and 324 defines a corresponding space with the base insole 298 such that the foot supports 317, 318, 319, and 320 may flex downwardly under a predetermined foot pressure providing a suspension effect to reduce shock and provide selected support to the wearer's foot, and providing greater comfort during walking, jogging, or running. The predetermined foot pressure of each foot support 317, 318, 319, and 320 is controlled by using at least one base insole strength modifying feature, such as adding one or more slots 325 through metatarsal arch support 317, the mid-portion 306, and the lateral cuboid support 318. The base insole 298 includes a downwardly extending lateral pivot 326 having a lowermost portion which is at a position relative to a bottom plane (not shown) of the base insole 298 preferably either slightly above or even with the bottom plane. The lateral pivot 326 assists in guiding the foot medially to the big toe for toe-off during striding and is used to adjust supination of the foot during striding.

Referring to FIGS. 16-18, therein is shown a third embodiment user-customizable insole 328. The insole 328 includes a third version upper insole 330 of full length and the first version base insole 24, and is shown as used with a third version insole customizing kit 332.

The upper insole 330 is relatively soft providing cushioning for the foot and shaped to fit into the footwear. The upper insole 330 is of a flexible sandwiched construction, comprising a flexible cloth sheet 334 bonded to a formed insole shell 335. The cloth sheet 334 is preferably made of an abrasion resistant material, such as synthetic fabric, nylon, dacron, felt, cloth, or the like. The cloth sheet 334 includes a integral formed toe grip 336 which extends laterally thereacross so as to be disposed adjacent and below the sulcus cavity of the wearer's foot. The toe grip 336 and those which follow provide a place for the toes of the wearer to grip the upper surface of the insole to maintain positioning thereof and a secure feeling for the wearer. The insole shell 335 is made of a resilient material such as EVA polyethylene shaped like the plantar surface of the foot and includes a molded-in toe grip 340 which matingly engages toe grip 336. A lower surface 342 of cloth sheet 334 is adhesively bonded to an upper surface 344 of insole shell 335 to form the upper insole 330, having an upper surface 346 adapted to engage the plantar surface of the wearer's foot, and a lower surface 348 adapted to fit to the base insole 24. The upper insole 330 may be a substantially flat sheet which forms to the contours of the base insole 24 or may be molded with contours to match the contours of the plantar surface of the foot and of the base insole 24. The upper insole 330 includes a forefoot portion 352 positionable underneath the metatarsal bones of the wearer's foot beginning at a frontal area 353 of the upper insole 330 comprising a toe portion 354 positionable under the phalanges of the foot. A mid-portion 355 of the upper insole 330 interconnects the forefoot portion 352 to a heel portion 356. The heel portion 356 is positionable under the calcaneus bone of the wearer's foot has a concave heel cup 357 to match the contour of the heel of the wearer's foot and a flat bottom 358 opposite heel cup 357 to interface with the base insole 24 and adapted to facilitate placement of intrinsic heel adjustment devices into the base insole 24 such as of the type described subsequently. The length of the upper insole 330 is optionally of two-thirds length as without a forward portion 360, as shown by the dotted line which is a trim line, a perforated line to facilitate breaking therealong, or showing a front edge if upper insole 330 is initially manufactured as two-thirds length, which then extends from the heel to the first metatarsals.

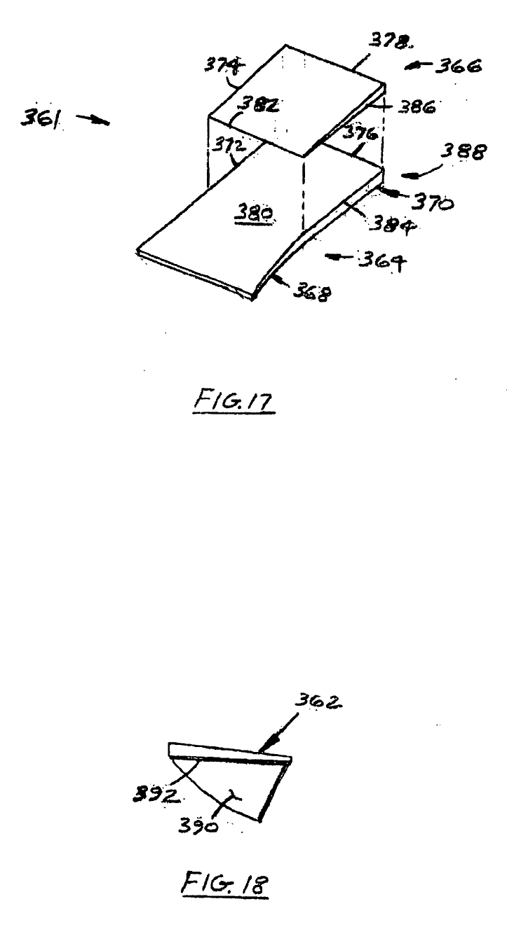

The third version insole customizing kit 332 includes a forefoot medial wedge 361 and a medial half heel wedge 362. As shown in FIGS. 16 and 17, the forefoot medial wedge 361 comprises respective elongate and short thin foam wedges 364 and 366. The foam wedge 364 includes a wedge portion 368 and a flat portion 370. The foam wedges 364 and 366 are affixed together at respective edges 372 and 374, 376 and 378, and surface 380 and edge 382, with respective free edges 384 and 386 so as to form a first metatarsal head extension receiving pocket 388. The forefoot medial wedge 361 slips onto the first metatarsal head extension 65 to correct the initial foot position and toe-off during striding. As shown in FIGS. 16 and 18, the medial half heel wedge 362 is made of a resilient material, preferably a sheet foam, preferably a visco-elastic foam, with a peel-off backing sheet 390 exposes an adhesive layer 392 to secure medial half heel wedge 362 to flat bottom 62 within the heel cup 61. The medial half heel wedge 362 corrects the initial heel and foot position and during striding from heel strike through toe for increased heel stability and alignment.

Referring to FIG. 19, therein is shown a fourth embodiment user-customizable insole 396. The insole 396 includes the third version upper insole 330 and a sixth version base insole 398 of two-thirds length, which comprises the base insole 24 with the first metatarsal head extension 65 snipped off, and is shown as used with a contoured heel pad 400. The heel pad 400 is made of resilient material, preferably a sheet of foam, preferably a visco-elastic foam, and includes a peel-off backing sheet 402 that exposes an adhesive layer 404 to secure heel pad 400 to the flat bottom 62 within heel cup 61. The heel pad 400 cushions impacts on the heel during striding.

Referring to FIG. 20, therein is shown a fifth embodiment user-customizable insole 406. The insole 406 includes a fourth version upper insole 408 of full length and the first version base insole 24, and is shown as used with a lateral half heel wedge 409.

The upper insole 408 is relatively soft providing cushioning for the foot and shaped to fit into the footwear (not shown). The upper insole 408 is of a flexible monolithic construction made of a flexible resilient cushioning material, such as ethylene-vinyl acetate polymer (EVA), though other similar cushioning open or closed cell foams may be used. The length of the upper insole 408 extends from the heel to the toes of the foot. The upper insole 408 is shaped like the outline of the sole or plantar surface of the foot, having an upper surface 410 adapted to engage the plantar surface of the wearer's foot, and a lower surface 412 adapted to fit to the base insole 24. The upper insole 408 is molded with contours to match the contours of the plantar surface of the foot and of the base insole 24. The upper insole 408 includes a forefoot portion 413 positionable underneath the metatarsal bones of the wearer's foot beginning at a frontal area 415 of the upper insole 408 comprising a toe portion 416. A slightly curved integral toe grip 417 extends generally transversely across the toe portion 416. A mid-portion 418 of the upper insole 408 interconnects the forefoot portion 413 to a heel portion 419. The heel portion 419 is positionable under the calcaneus bone of the wearer's foot has a concave heel cup 420 to match the contour of the heel of the wearer's foot and a flat bottom 421 opposite heel cup 420 to interface with the base insole 24 and adapted to facilitate placement of intrinsic heel adjustment devices into the base insole 24. The toe portion 416 extends forwardly as part of the frontal area 415 of the upper insole 408 under the phalanges of the foot to provide cushioning. The length of the upper insole 408 is optionally of two-thirds length with a forward portion 422 removed, as shown by the dotted line which is a trim line, a perforated line to facilitate breaking therealong, or showing a front edge if upper insole 408 is initially manufactured as two-thirds length, which extends from the heel to behind the metatarsals.

The lateral half heel wedge 409 is made of resilient material, preferably a sheet of foam, preferably a visco-elastic foam, with a peel-off backing sheet 424 exposes an adhesive layer 426 to secure lateral half heel wedge 409 to the flat bottom 62 within heel cup 61. The lateral half heel wedge 409 corrects the initial heel and foot position and during striding from heel strike through toe for increased heel stability and alignment.

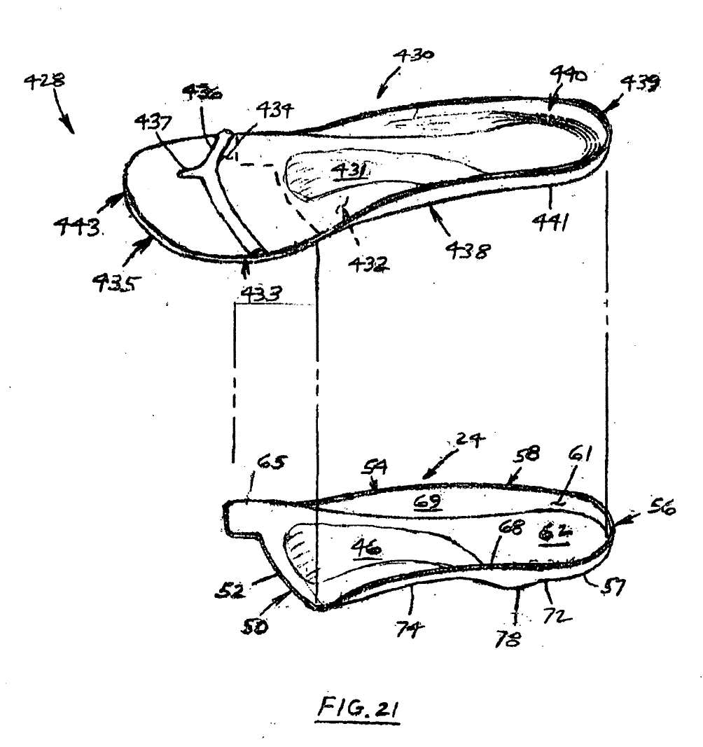

Referring to FIG. 21, therein is shown a sixth embodiment user-customizable insole 428. The insole 428 includes a fifth version upper insole 430 of full length and the first version base insole 24.

The upper insole 430 is relatively soft providing cushioning for the foot and shaped to fit into the footwear (not shown). The upper insole 430 is of a flexible monolithic construction made of a flexible resilient cushioning material, such as ethylene-vinyl acetate polymer (EVA), though other similar cushioning open or closed cell foams may be used. The length of the upper insole 430 extends from the heel to the toes of the foot. The upper insole 430 is shaped like the outline of the sole or plantar surface of the foot, having an upper surface 431 adapted to engage the plantar surface of the wearer's foot, and a lower surface 432 adapted to fit to the base insole 24. The upper insole 430 is molded with contours to match the contours of the plantar surface of the foot and of the base insole 24. The upper insole 430 includes a forefoot portion 433 positionable underneath the metatarsal bones of the wearer's foot beginning at a frontal area 434 of the upper insole 430 comprising a toe portion 435. A slightly curved integral toe grip 436 having a toe separator 437 extends generally transversely across the toe portion 435. A mid-portion 438 of the upper insole 430 interconnects the forefoot portion 433 to a heel portion 439. The heel portion 439 is positionable under the calcaneus bone of the wearer's foot has a concave heel cup 440 to match the contour of the heel of the wearer's foot and a flat bottom 441 opposite heel cup 440 to interface with the base insole 24 and adapted to facilitate placement of intrinsic heel adjustment devices into the base insole 24. A first metatarsal head extension 442 extends forwardly as part of the frontal area 434 of the upper insole 430 under the first metatarsal head of the wearer's foot to support the first metatarsal head. The length of the upper insole 430 is optionally of two-thirds length with a forward portion 443 removed, as shown by the dotted line which is a trim line, a perforated line to facilitate breaking therealong, or showing a front edge if upper insole 430 is initially manufactured as two-thirds length, which extends from the heel to behind the metatarsals.

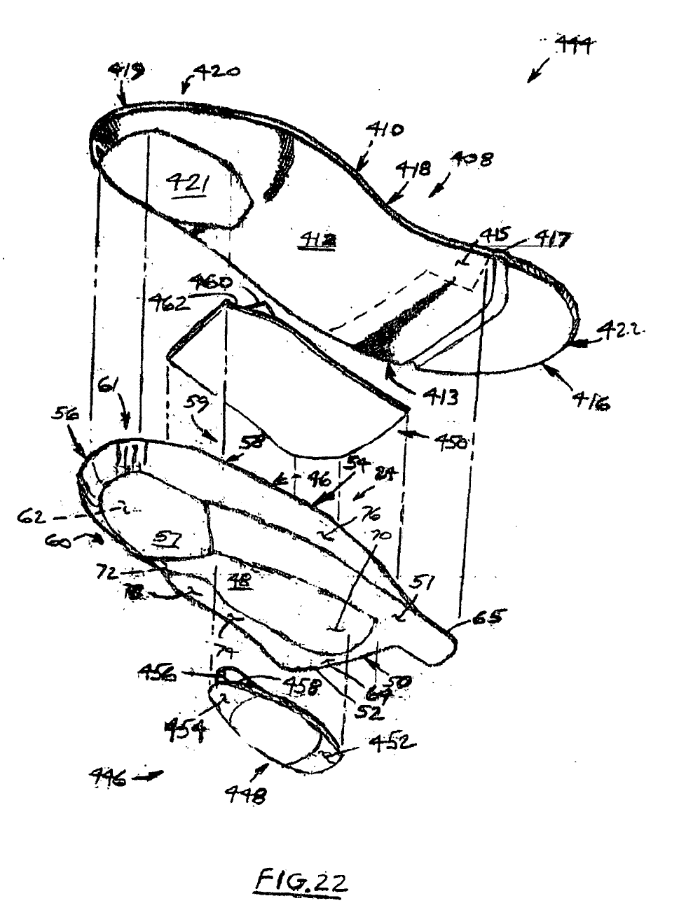

Referring to FIG. 22, therein is shown the seventh embodiment user-customizable insole 444 shown as used with a fourth version insole customizing kit 446. The insole 444 includes the fourth version upper insole 408 and the first version base insole 24.

The fourth version insole customizing kit 446 includes a metatarsal arch support pad 448 and an underfoot support pad 450. The metatarsal arch support pad 448 is made of a resilient material, preferably a rounded sheet of foam, preferably a visco-elastic foam, having respective tapered edges 452 and 454. A peel-off backing sheet 456 exposes an adhesive layer 458 to secure metatarsal arch support pad 448 to the elevation curve 70 below the metatarsal arch support 66 of the base insole 24 for increased metatarsal support. The underfoot support pad 450 is made of a resilient material, preferably a sheet of foam, preferably visco-elastic foam. A peel-off backing sheet 460 exposes an adhesive layer 462 to secure underfoot support pad 450 to the lower surface 48 of the base insole 24 for underfoot support. The underfoot support pad 450 acts to flesh out on the underfoot's surface and easily compresses. The support pads 448 and 450 may also be affixed to the base insole 24 using strips of conventional double-sided adhesive tape (not shown).

Referring to FIG. 23, therein is shown an eighth embodiment user-customizable insole 463. The insole 463 includes the fifth version upper insole 430 and a seventh version base insole 464 of two-thirds length.

The seventh version base insole 464 includes an upper surface 465 which is contoured to provide support to the plantar surface of the wearer's foot and a lower surface 466 adapted to engage the inside bottom of the footwear. The base insole 464 is about two-thirds the length of the foot, extending from the heel to the forefoot of a wearer's foot behind the metatarsal heads of the foot. The base insole 464 includes a forefoot portion 467 positionable underneath the metatarsal bones of the wearer's foot beginning at a frontal area 468 of the base insole 464 comprising a forward edge 469. A mid-portion 470 of the base insole 464 interconnects the forefoot portion 467 to a heel portion 471 positionable underneath the calcaneus bone of the wearer's foot and has a large flat bottom 472. A tapered perimeter wall 473 extends around heel portion 471 and forwardly along respective medial and lateral sides 474 and 475 of the base insole 464 defining a concave heel cup 476 with an large flat bottom 478. The perimeter wall 473 is highest about the heel portion 471, tapering downwardly through the mid-portion 470, and tapering away at about the forefoot portion 467. A first metatarsal head extension 480 extends forwardly as part of the frontal area 468 of the base insole 464 to support the first metatarsal head of the wearer's foot.

The base insole 464 has a built-in orthopedic contour, including a plurality of integral foot supports. An upwardly curved slotted metatarsal arch support 482 is formed in base insole 464 to support of the metatarsal bones of the foot. The metatarsal arch support 482 begins at the forward edge 469 of the forefoot portion 467 and includes one or more slots 484 therethrough which extend from the forward edge 469 rearwardly into the metatarsal arch support 482. Each slot 484 terminates at a circular end 488 which relieves stresses to prevent cracking of base insole 464. There are preferably four slots which are disposed so as to be between respective first, second, third, fourth, and fifth metatarsal bones of the wearer's foot. This allows downward flexing or a suspension effect of the base insole 464 under higher loads providing shock absorption and greater comfort during walking, jogging, or running. An upwardly curved lateral cuboid support 490 is formed in base insole 464 to support the cuboid bone of the foot. An upwardly curved lateral arch support 492 is formed in base insole 464 to support the lateral arch curve of the foot. An upwardly curved medial longitudinal arch support 494 is formed in base insole 464 to support the medial longitudinal arch curve of the foot. The medial longitudinal arch support 494 also provides support for the calcaneus bone of the foot in relation to the plantar fascia. The underside of the foot supports 482, 490, 492, and 494 comprise corresponding upwardly curved elevation curves 496, 498, 500, and 502 which are disposed above the inside bottom of the footwear. Each elevation curve 496,498, 500, and 502 defines a corresponding space (not shown) with the inside bottom of the footwear such that the foot supports 482, 490, 492, and 494 may flex downwardly under a predetermined foot pressure providing a suspension effect to reduce shock and provide selected support to the wearer's foot, and providing greater comfort during walking, jogging, or running. The predetermined foot pressure of each foot support 482, 490, 492, and 494 is controlled by using at least one base insole strength modifying feature. The base insole 464 includes a downwardly extending lateral pivot 511 having a lowermost portion which is at a position relative to a bottom plane (not shown) of the base insole 464 preferably either slightly above or even with the bottom plane. The lateral pivot 511 assists in guiding the foot medially to the big toe for toe-off during striding and is used to adjust supination of the foot during striding.

Referring to FIG. 24, therein is shown a ninth embodiment user-customizable insole 512. The insole 512 includes the fourth version cushioning upper insole 408 and an eighth version base insole 513 of two-thirds length.

The base insole 513 includes an upper surface 514 which is contoured to provide support to the plantar surface of the wearer's foot and a lower surface 515 adapted to engage the inside bottom of the footwear. The base insole 513 is about two-thirds the length of the foot, extending from the heel to the forefoot of a wearer's foot behind the metatarsal heads of the foot. The base insole 513 includes a forefoot portion 517 positionable underneath the metatarsal bones of the wearer's foot beginning at a frontal area 518 of the base insole 513 comprising a forward edge 519. A mid-portion 520 of the base insole 513 interconnects the forefoot portion 517 to a heel portion 521 positionable underneath the calcaneus bone of the wearer's foot and has a large flat bottom 522. A tapered perimeter wall 523 extends around heel portion 521 and forwardly along respective medial and lateral sides 524 and 525 of the base insole 513 defining a concave heel cup 526 with a large flat bottom 527. An integrally molded heel support 528 extends downwardly from the heel portion 521, the heel support 528 having a flat bottom 529 which engages the inside bottom of the footwear to stabilize the heel of the wearer's foot within the footwear. The heel support 528 is integrally molded of the same plastic such as polypropylene as the base insole 513, or of a resilient material such as rubber or neoprene molded onto the base insole 513 to cushion impact forces to the heel. The perimeter wall 523 is highest about the heel portion 521, tapering downwardly through the mid-portion 520, and tapering away at about the forefoot portion 517. The forefoot portion 517 includes a narrow flat support lip 530 disposed from the forward edge 519 rearwardly. A first metatarsal head extension 532 extends forwardly as part of the frontal area 518 of the base insole 513 to support the first metatarsal head of the wearer's foot.

The base insole 513 has a built-in orthopedic contour, including a plurality of integral foot supports. An upwardly curved metatarsal arch support 534 is formed in base insole 513 to support of the metatarsal bones of the foot. The metatarsal arch support 534 begins at the forward edge 519 of the frontal area 518. An upwardly curved lateral cuboid support 536 is formed in base insole 513 to support the cuboid bone of the foot. An upwardly curved lateral arch support 538 is formed in base insole 513 to support the lateral arch curve of the foot. An upwardly curved medial longitudinal arch support 540 is formed in base insole 513 to support the medial longitudinal arch curve of the foot. The medial longitudinal arch support 540 provides support for the calcaneus bone of the foot in relation to the plantar fascia. A downwardly curved longitudinal plantar fascia ligament support 542 provides support for the plantar fascia ligament. The underside of the upwardly curved foot supports 534, 536, 538, and 540 comprise corresponding upwardly curved elevation curves 543, 544, 546, and 548 which are disposed above the inside bottom of the footwear. Each elevation curve 543, 544, 546, and 548 defines a corresponding space (not shown) with the inside bottom of the footwear such that the foot supports 534, 536, 538, and 540 may flex downwardly under a predetermined foot pressure providing a suspension effect to reduce shock and provide selected support to the wearer's foot, and providing greater comfort during walking, jogging, or running. The predetermined foot pressure of each foot support 534, 536, 538, 540, and 542 is controlled by using at least one base insole strength modifying feature, such as adding round holes 550 through metatarsal arch support 534 and the longitudinal plantar fascia ligament support 542. The base insole 513 includes a downwardly extending lateral pivot 555 having a lowermost portion which is at a position relative to a bottom plane (not shown) of the base insole 513 preferably either slightly above or even with the bottom plane. The lateral pivot 555 assists in guiding the foot medially to the big toe for toe-off during striding and is used to adjust supination of the foot during striding.

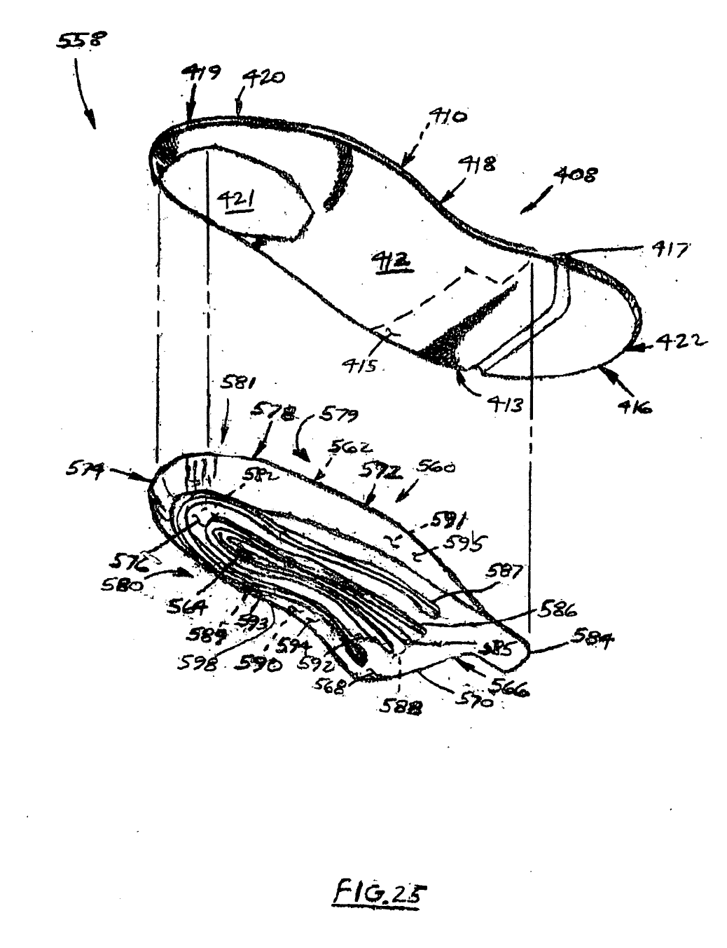

Referring to FIG. 25, therein is shown a tenth embodiment user-customizable insole 558. The insole 558 includes the fourth version upper insole 408 and a ninth version base insole 560 of two-thirds length.

The base insole 560 includes an upper surface 562 which is contoured to provide support to the plantar surface of the wearer's foot and a longitudinally ribbed lower surface 564 adapted to engage the inside bottom of the footwear. The base insole 560 is about two-thirds the length of the foot, extending from the heel to the forefoot of a wearer's foot behind the metatarsal heads of the foot. The base insole 560 includes a forefoot portion 566 positionable underneath the metatarsal bones of the wearer's foot beginning at a frontal area 568 of the base insole 560 comprising a forward edge 570. A mid-portion 572 of the base insole 560 interconnects the forefoot portion 566 to a heel portion 574 positionable underneath the calcaneus bone of the wearer's foot and has a large flat bottom 576. A tapered perimeter wall 578 extends around heel portion 574 and forwardly along respective medial and lateral sides 579 and 580 of the base insole 560 defining a concave heel cup 581 with a large flat bottom 582. The perimeter wall 578 is highest about the heel portion 574, tapering downwardly through the mid-portion 572, and tapering away at about the forefoot portion 566. A first metatarsal head extension 584 extends forwardly as part of the frontal area 568 of the base insole 560 to support the first metatarsal head of the wearer's foot to support the first metatarsal head.

The base insole 560 includes a plurality of spaced, integral longitudinal reinforcing support ribs 585, 586, and 587 which extend along the lower surface 564 of the base insole 560 from the forefoot portion 566 longitudinally rearward, curving one-hundred-eighty degrees under the heel portion 574, and returning to the forefoot portion 566 to stiffen the base insole 560. The support ribs 585, 586, and 587 provide reinforcement to maintain controlled support and flexing to the base insole 560, primarily in the longitudinal direction, by resisting to the desired extent downward deflection of the base insole 560, and help prevent cracking of the base insole 560. The number, positioning, and cross-sectional area of the support ribs 585, 586, and 587 may be varied depending on the support characteristics desired of the base insole 560. For example, the support ribs 585, 586, and 587 might comprise other than flat strips integrally formed with the base insole 560 as shown, and might also be trimmed to adjust the amount of support provided to the base insole 560. The ribs 585, 586, and 587 may also be made from a resilient shock absorbing material such as compressed EVA or polyurethane the hardness and density selection range will determine the shock absorption.

The base insole 560 has a built-in orthopedic contour, including a plurality of integral foot supports. An upwardly curved metatarsal arch support 588 is formed in base insole 560 to support of the metatarsal bones of the foot. The metatarsal arch support 588 begins at the forward edge 570 of the forefoot portion 566. An upwardly curved lateral cuboid support 589 is formed in base insole 560 to support the cuboid bone of the foot. An upwardly curved lateral arch support 590 is formed in base insole 560 to support the lateral arch curve of the foot. An upwardly curved medial longitudinal arch support 591 is formed in base insole 560 to support the medial longitudinal arch curve of the foot. The medial longitudinal arch support 591 also provides support for the calcaneus or heel bone of the foot in relation to the plantar fascia. The underside of the foot supports 588, 589, 590, and 591 comprise corresponding upwardly curved elevation curves 592, 593, 594, and 595 which are disposed above the inside bottom of the footwear. Each elevation curve 592, 593, 594, and 595 defines a corresponding space (not shown) with the inside bottom of the footwear such that the foot supports 588, 589, 590, and 591 may flex downwardly under a predetermined foot pressure providing a suspension effect to reduce shock and provide selected support to the wearer's foot, and providing greater comfort during walking, jogging, or running. The predetermined foot pressure of each foot support 588, 589, 590, and 591 is controlled by using at least one base insole strength modifying feature. The base insole 560 includes a downwardly extending lateral pivot 598 having a lowermost portion which is at a position relative to a bottom plane (not shown) of the base insole 560 preferably either slightly above or even with the bottom plane. The lateral pivot 598 assists in guiding the foot medially to the big toe for toe-off during striding and is used to adjust supination of the foot during striding.

Referring to FIG. 26, therein is shown a eleventh embodiment user-customizable insole 600. The insole 600 includes the third version cushioning upper insole 330 and a tenth version base insole 602 of full length, and is shown used with a gel heel pad 604.

The base insole 602 includes an upper surface 605 which is contoured to provide support to the plantar surface of the wearer's foot and a lower surface 606 adapted to engage the inside bottom of the footwear. The base insole 602 is of the full length of the foot, extending from the heel to the ends of the toes of the wearer's foot. The base insole 602 includes a forefoot portion 608 positionable underneath the metatarsal bones of the wearer's foot beginning at a frontal area 610 of the base insole 602 comprising a toe portion 612. A mid-portion 614 of the base insole 602 interconnects the forefoot portion 608 to a heel portion 616 positionable underneath the calcaneus bone of the wearer's foot and has a large flat bottom 618. A tapered perimeter wall 620 extends around heel portion 616 and forwardly along respective medial and lateral sides 621 and 622 of the base insole 602 defining a concave heel cup 623 with a large flat bottom 624. The perimeter wall 620 is highest about the heel portion 616, tapering downwardly through the mid-portion 614, and tapering away at about the forefoot portion 608. About a front most one-third 625 of the base insole 602 is thinner than a remaining portion 626 of the base insole 602, being molded at a thickness that allows for a live hinge per standard engineering guidelines of the material used.

The base insole 602 has a built-in orthopedic contour, including a plurality of integral foot supports. The base insole 602 is relatively thin and of a generally uniform thickness, with an average thickness of between about 0.032 and 0.096 inch. An upwardly curved metatarsal arch support 627 is formed in base insole 602 to support of the metatarsal bones of the foot. The metatarsal arch support 627 begins at the frontal area 610 at the junction of the forefoot portion 608 and the toe portion 612. An upwardly curved lateral cuboid support 628 is formed in base insole 602 to support the cuboid bone of the foot. An upwardly curved lateral arch support 629 is formed in base insole 602 to support the lateral arch curve of the foot. An upwardly curved medial longitudinal arch support 630 is formed in base insole 602 to support the medial longitudinal arch curve of the foot. The medial longitudinal arch support 630 provides support for the calcaneus bone of the foot in relation to the plantar fascia. The underside of the foot supports 627, 628, 629, and 630 comprise corresponding upwardly curved elevation curves 631, 632, 633, and 634 which are disposed above the inside bottom of the footwear. Each elevation curve 631, 632, 633, and 634 defines a corresponding space (not shown) with the inside bottom of the footwear such that the foot supports 627, 628, 629, and 630 may flex downwardly under a predetermined foot pressure providing a suspension effect to reduce shock and provide selected support to the wearer's foot, and providing greater comfort during walking, jogging, or running. The predetermined foot pressure of each foot support 627, 628, 625, and 630 is controlled by using at least one base insole strength modifying feature. The base insole 602 includes a downwardly extending lateral pivot 636 having a lowermost portion which is at a position relative to a bottom plane (not shown) of the base insole 602 preferably either slightly above or even with the bottom plane. The lateral pivot 636 assists in guiding the foot medially to the big toe for toe-off during striding and is used to adjust supination of the foot during striding.

The gel heel pad 604 may be any one of the conventional gel-type pads available, preferably with a peel-off backing sheet 638 that exposes an adhesive layer 640 to secure gel heel pad 604 within heel cup 623. The gel heel pad 604 cushions the heel from impact forces during striding and may be utilized to treat and prevent heel spurs.

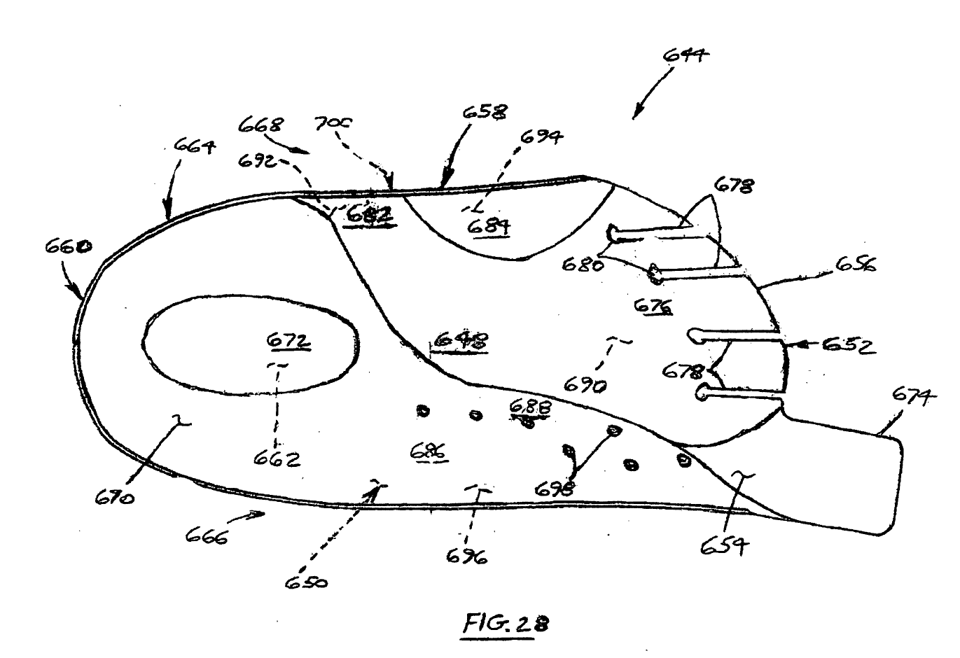

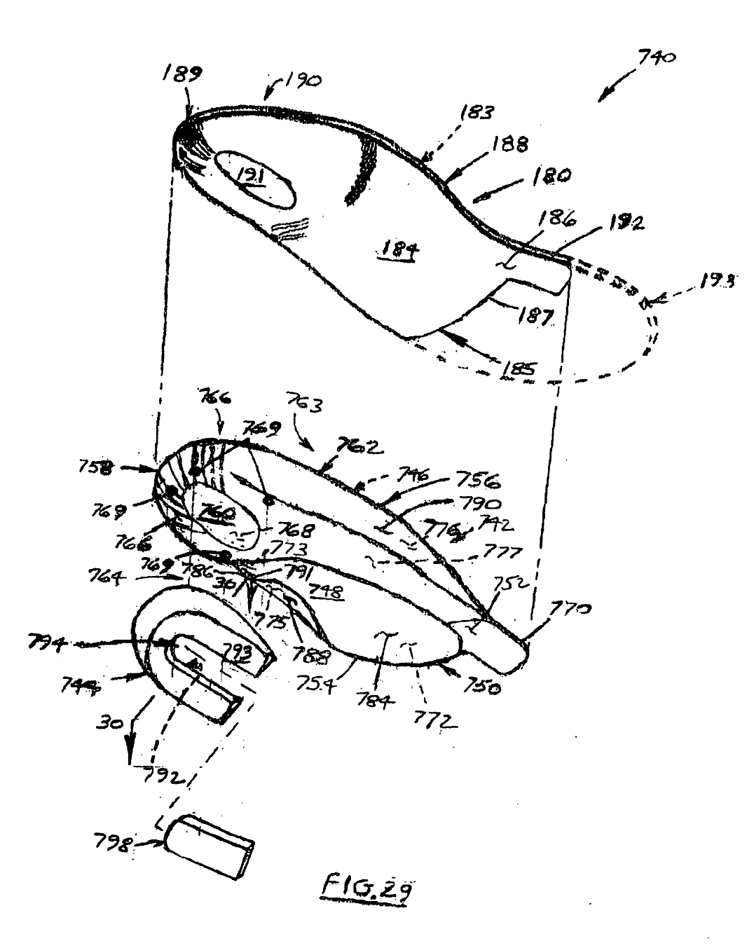

Referring to FIGS. 27 and 28, therein is shown a twelfth embodiment user-customizable insole 642 which includes the second version upper insole 180 and an eleventh version base insole 644 having a separately molded U-shaped heel support 646 affixed thereto, and is shown as used with a fifth version insole customizing kit 647. The length of the upper insole 180 is optionally of full length including the forward portion 193, as shown by the dotted lines, extending from the heel to the toes of the foot.

The base insole 644 includes an upper surface 648 which is contoured to provide support to the plantar surface of the wearer's foot and a lower surface 650 adapted to engage the inside bottom of the footwear. The base insole 644 is about two-thirds the length of the foot, extending from the heel to the forefoot of a wearer's foot behind the metatarsal heads of the foot. The base insole 644 includes a forefoot portion 652 positionable underneath the metatarsal bones of the wearer's foot beginning at a frontal area 654 of the base insole 644 comprising a forward edge 656. A mid-portion 658 of the base insole 644 interconnects the forefoot portion 652 to a heel portion 660 positionable underneath the calcaneus bone of the wearer's foot and has an oval flat bottom 662. A tapered perimeter wall 664 extends around heel portion 660 and forwardly along respective medial and lateral sides 666 and 668 of the base insole 644 defining a concave heel cup 670 with an oval flat bottom 672. The perimeter wall 664 is highest about the heel portion 660, tapering downwardly through the mid-portion 658, and tapering away at about the forefoot portion 652. A first metatarsal head extension 674 extends forwardly as part of the frontal area 654 of the base insole 644 to support the first metatarsal head of the wearer's foot.

The base insole 644 has a built-in orthopedic contour, including a plurality of integral foot supports. An upwardly curved slotted metatarsal arch support 676 is formed in base insole 644 to support of the metatarsal bones of the foot. The metatarsal arch support 676 begins at the forward edge 656 of the forefoot portion 652 and includes one or more slots 678 therethrough which extend from the forward edge 656 rearwardly into the metatarsal arch support 676. Each slot 678 terminates at a circular end 680 which relieves stresses to prevent cracking of base insole 644. There are preferably four slots which are disposed so as to be between respective first, second, third, fourth, and fifth metatarsal bones of the wearer's foot. This allows downward flexing or a suspension effect of the base insole 644 under higher loads providing shock absorption and greater comfort during walking, jogging, or running. An upwardly curved lateral cuboid support 682 is formed in base insole 644 to support the cuboid bone of the foot. An upwardly curved lateral arch support 684 is formed in base insole 644 to support the lateral arch curve of the foot. An upwardly curved medial longitudinal arch support 686 is formed in base insole 644 to support the medial longitudinal arch curve of the foot. The medial longitudinal arch support 686 provides support for the calcaneus bone of the foot in relation to the plantar fascia. A downwardly curved longitudinal plantar fascia ligament support 688 provides support for the plantar fascia ligament. The underside of the upwardly curved foot supports 676, 682, 684, and 686 comprise corresponding upwardly curved elevation curves 690, 692, 694, and 696 which are disposed above the inside bottom of the footwear. Each elevation curve 690, 692, 694, and 696 defines a corresponding space (not shown) with the inside bottom of the footwear such that the foot supports 676, 682, 684, and 686 may flex downwardly under a predetermined foot pressure providing a suspension effect to reduce shock and provide selected support to the wearer's foot, and providing greater comfort during walking, jogging, or running. The predetermined foot pressure of each foot support 676, 682, 684, and 686 is controlled using at least one base insole strength modifying feature, such as adding round holes 698 through the longitudinal plantar fascia ligament support 688. The base insole 644 includes a downwardly extending lateral pivot 700 having a lowermost portion which is at a position relative to a bottom plane (not shown) of the base insole 644 preferably either slightly above or even with the bottom plane. The lateral pivot 700 assists in guiding the foot medially to the big toe for toe-off during striding and is used to adjust supination of the foot during striding.

The heel support 646 is of a U-shape including a concave upper surface 702 adapted to be affixed to the heel portion 660 of the base insole 644. The heel support 646 includes a flat bottom 704. The heel support 646 is bonded the heel portion 660 of the base insole 644 extending downwardly therefrom and forwardly along the medial side 666 and lateral side 668 of the base insole 644 forming an open space 706 under the lower surface 650 of the base insole 644. The open space 706 permits the base insole 644 to flex downwardly into the open space 706 to cushion heel impacts. The flat bottom 704 engages the inside bottom of the footwear to stabilize the heel of the wearer's foot within the footwear. The heel support 646 is made of a resilient material, preferably a sheet of foam, preferably a visco-elastic foam, though rubber or neoprene may be used to cushion impact forces to the heel. The open space 706 under the lower surface 650 of the base insole 644 may be filled with a plug (not shown) of resilient filler material such as EVA, rubber, or neoprene to adjust the support provided by heel support 646.

The fifth version insole customizing kit 647 includes the heel wedge 409, a metatarsal arch support pad 708, and a pivot pad 710. The metatarsal arch support pad 708 is made of a resilient material, preferably a rounded triangular sheet of foam, preferably a visco-elastic foam, having a peel-off backing sheet 712 which exposes an adhesive layer 714 to secure metatarsal arch support pad 708 to the elevation curve 690 to back-fill the open space below the metatarsal arch support 676 and provide extra support for the metatarsal arch support 676. The pivot pad 710 is made of a resilient material, preferably a rectangular sheet of foam, preferably a visco-elastic foam, having a peel-off backing sheet 716 which exposes an adhesive layer 718 to secure pivot pad 710 to the lateral pivot 700. The pivot pad 710 makes the elevation of the lateral pivot 700 adjustable. The heel wedge 409, the metatarsal arch support pad 708, and the pivot pad 710 may also be affixed to the base insole 644 using strips of conventional double-sided adhesive tape (not shown). The base insole 644 preferably includes positioning indicia 720 etched or printed thereon providing placement instructions and which marks the locations for placement of the heel wedge 409, the metatarsal arch support pad 708, and the pivot pad 710 to assure correct placement thereof on base insole 644.

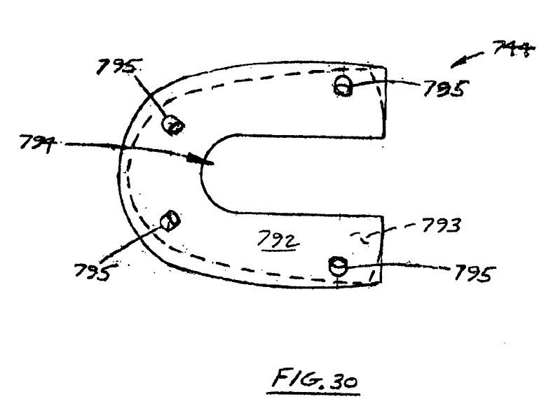

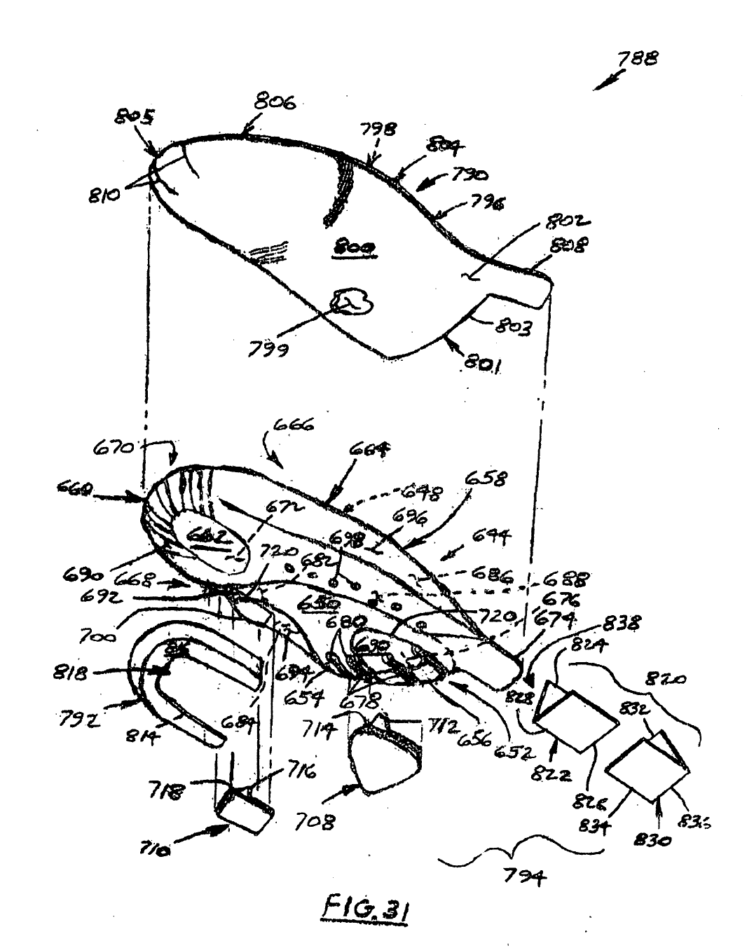

Referring to FIGS. 29 and 30, therein is shown a thirteenth embodiment user-customizable insole 740 which includes the second version upper insole 180, and a twelfth version base insole 742 of two-thirds length with a heel support 744 affixed thereto. The length of the upper insole 180 is optionally of full length including the forward portion 193, as shown by the dotted lines, which extends from the heel to the toes of the foot.

The base insole 742 includes an upper surface 746 which is contoured to provide support to the plantar surface of the wearer's foot and a lower surface 748 adapted to engage the inside bottom of the footwear. The base insole 742 is about two-thirds the length of the foot, extending from the heel to the forefoot of a wearer's foot behind the metatarsal heads of the foot. The base insole 742 includes a forefoot portion 750 positionable underneath the metatarsal bones of the wearer's foot beginning at a frontal area 752 of the base insole 742 comprising a forward edge 754. A mid-portion 756 of the base insole 742 interconnects the forefoot portion 750 to a heel portion 758 positionable underneath the calcaneus bone of the wearer's foot and has a large flat bottom 760. A tapered perimeter wall 762 extends around heel portion 758 and forwardly along respective medial and lateral sides 763 and 764 of the base insole 742 defining a concave heel cup 766 with an oval flat bottom 768. The perimeter wall 762 is highest about the heel portion 758, tapering downwardly through the mid-portion 756, and tapering away at about the forefoot portion 750. A plurality of heel locating holes 769 extend through the heel cup 766. A first metatarsal head extension 770 extends forwardly as part of the frontal area 752 of the base insole 742 to support the first metatarsal head of the wearer's foot.

The base insole 742 has a built-in orthopedic contour, including a plurality of integral foot supports. An upwardly curved metatarsal arch support 772 is formed in base insole 742 to support of the metatarsal bones of the foot. The metatarsal arch support 772 begins at the forward edge 754 of the forefoot portion 750. An upwardly curved lateral cuboid support 773 is formed in base insole 742 to support the cuboid bone of the foot. An upwardly curved lateral arch support 775 is formed in base insole 742 to support the lateral arch curve of the foot. An upwardly curved medial longitudinal arch support 776 is formed in base insole 742 to support the medial longitudinal arch curve of the foot. The medial longitudinal arch support 776 provides support for the calcaneus bone of the foot in relation to the plantar fascia. A downwardly curved longitudinal plantar fascia ligament support 777 provides support for the plantar fascia ligament. The underside of the upwardly curved foot supports 772, 773, 775, and 776 comprise corresponding upwardly curved elevation curves 784, 786, 788, and 790 which are disposed above the inside bottom of the footwear. Each elevation curve 784, 786, 788, and 790 defines a corresponding space (not shown) with the inside bottom of the footwear such that the foot supports 772, 773, 775, and 776 may flex downwardly under a predetermined foot pressure providing a suspension effect to reduce shock and provide selected support to the wearer's foot, and providing greater comfort during walking, jogging, or running. The predetermined foot pressure of each foot support 772, 773, 775, and 776 is controlled by using at least one base insole strength modifying feature. The base insole 742 includes a downwardly extending lateral pivot 791 having a lowermost portion which is at a position relative to a bottom plane (not shown) of the base insole 742 preferably either slightly above or even with the bottom plane. The lateral pivot 791 assists in guiding the foot medially to the big toe for toe-off during striding and is used to adjust supination of the foot during striding.

The heel support 744 is of a U-shape including a concave upper surface 792 adapted to be affixed to the heel portion 758 of the base insole 742. The heel support 744 includes a flat bottom 793. The heel support 744 is bonded the heel portion 758 of the base insole 742 extending downwardly therefrom and forwardly along the medial side 763 and lateral side 764 of the base insole 742 forming an open space 794 under the lower surface 748 of the base insole 742. A plurality of heel locating pins 795 of the heel support 744 are adapted to engage the heel locating holes 769 of the heel cup 766 to correctly position the heel support 744 on the base insole 742. Alternatively, the heel support 744 can have the heel locating holes 769 and the heel cup 766 have the mating heel locating pins 795. The open space 794 permits the base insole 742 to flex downwardly into the open space 794 to cushion heel impacts. The flat bottom 793 engages the inside bottom of the footwear to stabilize the heel of the wearer's foot within the footwear. The heel support 744 is made of a resilient material, preferably visco-elastic foam, though other such material as rubber or neoprene may be used to cushion impact forces to the heel. The open space 794 may be filled with a plug 798 of resilient filler material such as EVA, rubber, or neoprene to adjust the support provided by heel support 744.

Referring to FIG. 31, therein is shown a fourteenth embodiment user-customizable insole 788 which includes a seventh version upper insole 790 of two-thirds length, the eleventh version base insole 644 with a heel support 792 affixed thereto, and is shown as used with a sixth version insole customizing kit 794.

The upper insole 790 is relatively soft providing cushioning for the foot and shaped to fit into the footwear (not shown). The upper insole 790 is of a cloth construction with a length of the upper insole 790 extends from the heel to the toes of the foot. The upper insole 790 is shaped like the outline of the sole or plantar surface of the foot, comprising a flexible cloth sheet 796 made of an abrasion resistant material, such as synthetic fabric, nylon, dacron, felt, cloth, or the like. The upper insole 790 has an upper surface 798 and a lower surface 800 covered with an adhesive layer 802. The upper insole 790 includes a heel portion 804, a forefoot portion 806, and a first metatarsal head extension 808. The heel portion 804 is formed by a plurality of pie-shaped slits 810 which are closed to form a concave shape in heel portion 804. The length of the upper insole 790 extends from the heel to the forefoot of a wearer's foot (not shown) behind the metatarsal heads of the foot. The lower surface 800 of the upper insole 790 is bonded to the upper surface 648 of base insole 644 using adhesive layer 802, which may be initially covered by a protective sheet (not shown).

The heel support 792 is of a U-shape including a concave upper surface 814 which is bonded to the lower surface 650 at the heel cup 670 of base insole 644. The heel support 792 includes a flat bottom 816 and a center space 818. The heel support 792 is made of a resilient material, preferably a visco-elastic foam, or though other such material as rubber or neoprene may be used to cushion impact forces to the heel. The center space 818 provides softer shock absorption by allowing the lower surface 650 to deflect downwardly thereinto. The center space 818 may be filled with a plug similar to plug 798.

The upper insole 790 is preferably completely affixed to the base insole 644 such that no intrinsic adjustments fit therebetween.

The sixth version insole customizing kit 794 includes a second version slip-on first metatarsal head boot 820, the metatarsal arch support pad 708, and the pivot pad 710. The boot 820 preferably comprises a folded inner sheet 822 made of thin sheet plastic having a pair of halves 824 and 826 interconnected at a fold line 828, and a folded outer sheet 830 preferably made of the same abrasion resistant material as the cloth sheet 796 of the upper insole 790, having a pair of halves 832 and 834 interconnected at a fold line 836. The halves 832 and 834 of outer sheet 830 are adhesively bonded to halves 824 and 826 of inner sheet 822 to form a first metatarsal head extension receiving pocket 838. The boot 820 slips onto the first metatarsal head extension 674 and may be made in different thicknesses as required to control pronation at toe off or standing. The metatarsal arch support pad 708 has the peel-off backing sheet 712 which exposes the adhesive layer 714 to secure metatarsal arch support pad 708 to the elevation curve 690 to back-fill the open space below the metatarsal arch support 676 and provide extra support for the metatarsal arch support 676. The pivot pad 710 has the peel-off backing sheet 716 which exposes the adhesive layer 718 to secure pivot pad 710 to the lateral pivot 700 of base insole 644. The boot 820, the metatarsal arch support pad 708, and the pivot pad 710 may also be affixed to the base insole 644 using strips of conventional double-sided adhesive tape (not shown). The base insole 644 preferably includes the positioning indicia 720 etched or printed thereon providing placement instructions and which marks the locations for placement of the metatarsal arch support pad 708 and the pivot pad 710 to assure correct placement thereof on base insole 644.

Referring to FIG. 32, therein is shown a fifteenth embodiment user-customizable insole 840 which includes a full length insole 842 and an adjustable toe grip kit 844. The insole 842 is relatively soft providing cushioning for the foot and shaped to fit into the footwear (not shown). The insole 842 is of a semi-rigid sandwiched construction with a length which extends from the heel to the toes of the foot. The insole 842 is shaped like the outline of the plantar surface of the foot, comprising a flexible cloth sheet 846 which is bonded to a contoured foam body 847 shaped like the plantar surface of the foot. The insole 842 has an upper surface 848 adapted to engage the plantar surface of the wearer's foot, and a flat lower surface 850 adapted to fit onto the inside bottom of the footwear. The insole 842 is molded with contours to match the contours of the plantar surface of the foot. The insole 842 includes a forefoot portion 852 positionable underneath the metatarsal bones of the wearer's foot beginning at a frontal area 854 of the insole 842 comprising a toe portion 856. A mid-portion 858 of the insole 842 interconnects the forefoot portion 852 to a heel portion 860. The heel portion 860 is positionable under the calcaneus bone of the wearer's foot has a concave heel cup 862 to match the contour of the heel of the wearer's foot. The insole 842 has a built-in orthopedic contour which includes a metatarsal arch support 864, a lateral cuboid support 866, a lateral arch support 868, and a medial longitudinal arch support 870. The insole 842 includes a plurality of perforations 871 therethrough for heat dissipation.

The toe grip kit 844 comprises a toe grip 872 and an alternate toe grip 874. The toe grip 872 includes an elongate, slightly curved body 876 adapted to extend laterally across the toe portion 856 of the insole 842 adjacent and under the sulcus cavity of the wearer's foot, and a curved, triangular toe separator 878. The toe grip 872 comprises a resilient soft to semi-rigid material, preferably a soft visco-elastic foam layer 880 to provide more comfort to the foot, onto which respective upper and lower cloth sheets 882 and 883 are bonded. A plurality of peel-off upper backing sheets 884 expose corresponding narrow upper adhesive layers 886 adapted to temporarily secure toe grip 872 to the underside of the toes at the sulcus cavity of the foot. A peel-off lower backing sheet 888 exposes a wide lower adhesive layer 890 to semi-permanently secure toe grip 872 to toe portion 856, the upper adhesive layers 886 having a lesser adhesion or affinity to the underside of the toes than the lower adhesive layer 890 has to the upper surface 848 of the insole 842. This difference in affinity may be achieved by the combined surface area of the body 876 covered by the upper adhesive layers 886 being smaller than a combined surface area of the body 876 covered by the lower adhesive layer 890. Alternatively, or in combination with surface area, the upper adhesive layers 886 may have lesser adhesion characteristics than the lower adhesive layer 890.

The toe grip 872 is used by the wearer stripping off the upper backing sheets 884 covering the upper adhesive layers 886 and pressing the toe grip 872 against the underside of the toes in the sulcus cavity of the foot. The upper adhesive layers 886 temporarily attach the toe grip 872 in place under the toes until the foot is placed onto the insole 842. The wearer then strips off the lower backing sheet 888 covering the lower adhesive layer 890, positions the foot over the insole 842, and steps the foot down onto insole 842 such that the lower adhesive layer 890 affixes the toe grip 872 to insole 842 in the proper position for the wearer's foot. When the wearer now steps their foot away from the insole 842 or manually pulls the insole 842 away from their foot, the upper adhesive layers 886 separate from the toes due to less adhesion thereto. The toe grip 872 remains attached to the upper surface 848 of insole 842 such that the toe grip attaches to the upper surface in an exact position when body weight is applied and releases from the toes when the foot is removed therefrom. The wearer may slightly move the toe grip 872 as needed to fine tune the position on the insole 842 to maximize foot comfort. Double-sided adhesive tape (not shown) may also be utilized in place of the backing sheets 884 and 888, and the adhesive layers 886 and 890, preferably with a narrower double-sided adhesive tape being used for temporarily securing the toe grip 872 to the toes than to permanently secure the toe grip 872 to the insole 842.

The alternate toe grip 874 includes an elongate, slightly curved body 892 adapted to extend laterally across the toe portion 856 of the insole 842 adjacent and under the sulcus cavity of the wearer's foot, but without a toe separator 878. The toe grip 874 is preferably of similar construction to toe grip 872, including a resilient soft to semi-rigid material, preferably a soft visco-elastic foam layer 894 to provide more comfort to the foot, onto which respective upper and lower cloth sheets 896 and 897 are bonded. A single peel-off upper backing sheet 898 exposes a narrow upper adhesive layer 900 adapted to temporarily secure toe grip 874 to the underside of the toes at the sulcus cavity of the foot. A peel-off lower backing sheet 902 exposes a wide lower adhesive layer 904 to semi-permanently secure toe grip 874 to toe portion 856, the upper adhesive layer 900 having a lesser affinity to the underside of the toes than the lower adhesive layer 904 has to the upper surface 848 of the insole 842. This difference in affinity is as described for the toe grip 872. The toe grip 874 is used by the wearer in the same manner as toe grip 872.

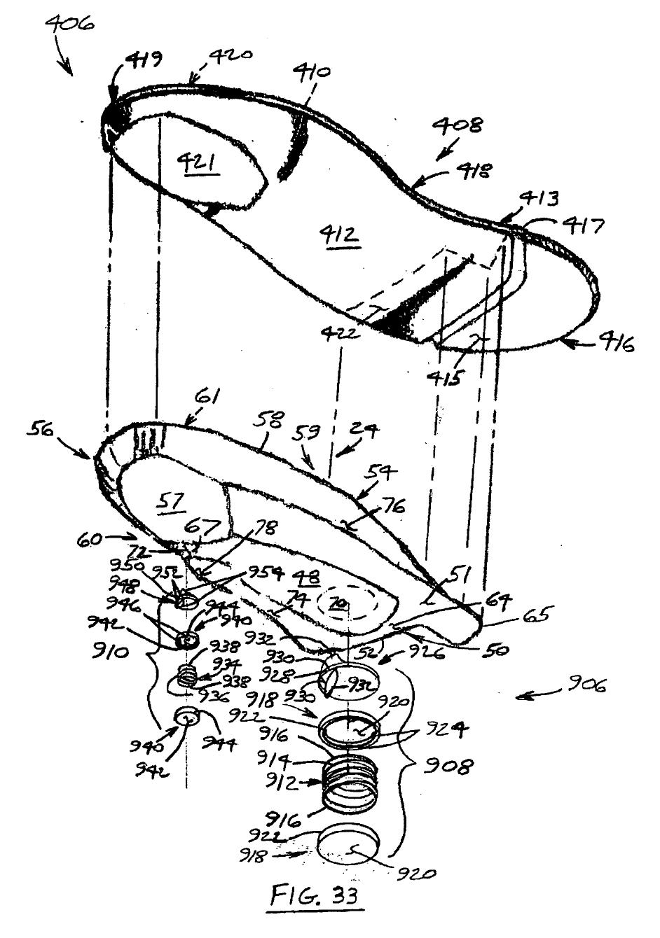

Referring to FIG. 33, therein is shown the fifth embodiment user-customizable insole 406 shown as used with a seventh version insole customizing kit 906. Kit 906 includes insole adjustment devices in the form of a metatarsal arch adjustment device comprising a metatarsal arch spring support 908 and a lateral cuboid adjustment device comprising a lateral cuboid spring support 910 to provide custom comfort and support to the wearer's foot.

Metatarsal arch spring support 908 includes a compression spring 912 made of a spring wire 914 and having opposite ends 916, one of the ends 916 being securable to the elevation curve 70 below the metatarsal arch support 66. Spring wire 914 is preferably of a diameter of between about 0.010 and 0.020 inch and the compression spring 912 preferably has between about four to six coils, a diameter of between about three-eighths to one-half inch, and fits (when slightly compressed) into the approximately one-quarter to three-eighths inch space below the elevation curve 70. Compression springs 912 with different spring constants may be used to provide a desired amount of support for the metatarsal arch support 66. Likewise, other types of compression springs 912 may be used such as conical springs, or non-round-coiled springs such as leaf springs, and the like. A pair of end caps 918 are adapted to be affixed such as by snap-fitting to respective of ends 916 of the compression spring 912 and be securable to the elevation curve 70. The end caps 918 spread out the force of compression spring 912 on the base insole 24 to provide comfortable, non-bulging foot support and to minimize wear to the wearer's shoe. The end caps 918 are preferably molded of a plastic material such as polyvinyl chloride, polyethylene, or polypropylene, each including a circular end wall 920, a dependent circular peripheral wall 922 adapted to closely fit about respective of ends 916, and a plurality of resilient retaining tabs 924. The end wall 920 and peripheral wall 922 are thin, preferably about one millimeter thick, so as to act as a spring. The tabs 924 extend radially inwardly from peripheral wall 920 opposite the end wall 920 and are adapted to deform sufficiently to allow wire 914 to pass thereby and retain one of ends 916 disposed within peripheral wall 922. The end walls 920 of end caps 918 are preferably slightly convex, though they may be flat, contoured, or the like to match the curvature of the elevation curve 70 or other surface they engage. One end cap 918 may also be integrally molded with the base insole 24. The ends 916 of compression spring 912 may be ground flat or otherwise designed to spread out the force of compression spring 912 on the base insole 24 to provide comfortable, non-bulging foot support and to minimize wear to the wearer's shoe such that end caps 918 eliminated.

The end cap 918 of metatarsal arch spring support 908 is secured to the elevation curve 70 using an adhesive attachment device such as an adhesive disk 926. Alternatively, other adhesive or non-adhesive attachment devices such as double-sided adhesive tape, an adhesive compound such as hardening liquid adhesive, or an adhesive layer temporarily covered with a peel-off backing sheet may be used. The opposite end cap 918 may be secured to the inside bottom of the footwear in a similar manner. Adhesive disk 926 is made from the same material as commercially available double sided adhesive foam core tape or similarly suitable material. The adhesive disk 926 includes a core 928 made of a resilient sheet foam material coated on opposite sides with respective adhesive layers 930. The adhesive disk 926 of metatarsal arch spring support 908 has a pair of peel-off backing disks 932 which expose the adhesive layers 930 to secure metatarsal arch spring support 908 to the elevation curve 70 to back-fill the open space below the metatarsal arch support 66 and provide extra support for the metatarsal arch support 66.

Lateral cuboid spring support 910 includes a compression spring 934 made of a spring wire 936 and having opposite ends 938, one of the ends 938 being securable to the elevation curve 72 below the lateral cuboid support 67. Spring wire 936 is preferably of a diameter of between about 0.010 and 0.020 inch and the compression spring 934 preferably has between about four to six coils, a diameter of about one-quarter inch, and fits (when slightly compressed) into the approximately one-quarter to three-eighths inch space below the elevation curve 72. Compression springs 934 with different spring constants may be used to provide a desired amount of support for the lateral cuboid support 67. Likewise, other types of compression springs 912 may be used such as conical springs, or non-round-coiled springs such as leaf springs, and the like. A pair of end caps 940 are adapted to be affixed such as by snap-fitting to respective of ends 938 of the compression spring 934 and be securable to the elevation curve 72. The end caps 940 spread out the force of compression spring 934 on the base insole 24 to provide comfortable, non-bulging foot support and to minimize wear to the wearer's shoe. The end caps 940 are preferably molded of a plastic material such as polyvinyl chloride, polyethylene, or polypropylene, each including a circular end wall 942, a dependent circular peripheral wall 944 adapted to closely fit about respective of ends 938, and a plurality of resilient retaining tabs 946. The end wall 942 and peripheral wall 944 are thin, preferably about one millimeter thick, so as to act as a spring. The tabs 946 extend radially inwardly from peripheral wall 944 opposite the end wall 942 and are adapted to deform sufficiently to allow wire 936 to pass thereby and retain one of ends 938 disposed within peripheral wall 944. The end walls 942 of end caps 940 are preferably slightly convex, though they may be flat, contoured, or the like to match the curvature of the elevation curve 72 or other surface they engage. One end cap 940 may also be integrally molded with the base insole 24. The ends 938 of compression spring 934 may be ground flat or otherwise designed to spread out the force of compression spring 934 on the base insole 24 to provide comfortable, non-bulging foot support and to minimize wear to the wearer's shoe such that end caps 940 eliminated.

The end cap 940 of lateral cuboid spring support 910 is secured to the elevation curve 72 using an adhesive attachment device such as an adhesive disk 948. Alternatively, other adhesive or non-adhesive attachment devices such as double-sided adhesive tape, an adhesive compound such as hardening liquid adhesive, or an adhesive layer temporarily covered with a peel-off backing sheet may be used. The opposite end cap 940 may be secured to the inside bottom of the footwear in a similar manner. Adhesive disk 948 is made from the same material as commercially available double sided adhesive foam core tape or similarly suitable material. The adhesive disk 948 includes a core 950 made of a resilient sheet foam material coated on opposite sides with respective adhesive layers 952. The adhesive disk 948 of lateral cuboid spring support 910 has a pair of peel-off backing disks 954 which expose the adhesive layers 952 to secure lateral cuboid spring support 908 to the elevation curve 72 to back-fill the open space below the lateral cuboid support 67 and provide extra support for the lateral cuboid support 67.

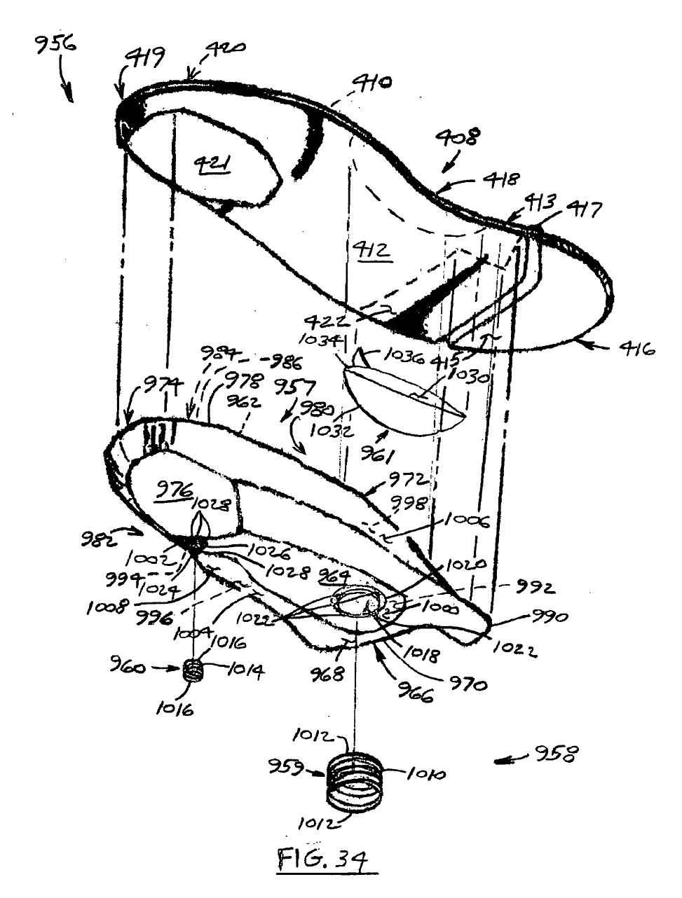

Referring to FIG. 34, therein is shown a sixteenth embodiment user-customizable insole 956 which includes the fourth version upper insole 408 and a thirteenth version base insole 957 of two-thirds length, and is shown as used with an eighth version insole customizing kit 958. Kit 958 includes insole adjustment devices in the form of a metatarsal arch adjustment device comprising a metatarsal arch compression spring 959, a lateral cuboid adjustment device in the form of a lateral cuboid compression spring 960, and a medial longitudinal arch support pad 961 to provide custom comfort and support to the wearer's foot.

The thirteenth version base insole 957 includes an upper surface 962 which is contoured to provide support to the plantar surface of the wearer's foot and a lower surface 964 adapted to engage the inside bottom of the footwear. The base insole 957 is about two-thirds the length of the foot, extending from the heel to the forefoot of a wearer's foot behind the metatarsal heads of the foot. The base insole 957 includes a forefoot portion 966 positionable underneath the metatarsal bones of the wearer's foot beginning at a frontal area 968 of the base insole 958 comprising a forward edge 970. A mid-portion 972 of the base insole 957 interconnects the forefoot portion 966 to a heel portion 974 positionable underneath the calcaneus bone of the wearer's foot and has a large flat bottom 976. A tapered perimeter wall 978 extends around heel portion 974 and forwardly along respective medial and lateral sides 980 and 982 of the base insole 957 defining a concave heel cup 984 with an large flat bottom 986. The perimeter wall 978 is highest about the heel portion 974, tapering downwardly through the mid-portion 972, and tapering away at about the forefoot portion 966. A first metatarsal head extension 990 extends forwardly as part of the frontal area 968 of the base insole 957 to support the first metatarsal head of the wearer's foot.

The base insole 957 has a built-in orthopedic contour, including a plurality of integral foot supports. An upwardly curved metatarsal arch support 992 is formed in base insole 957 to support the metatarsal bones of the foot. An upwardly curved lateral cuboid support 994 is formed in base insole 957 to support the cuboid bone of the foot. An upwardly curved lateral arch support 996 is formed in base insole 957 to support the lateral arch curve of the foot. An upwardly curved medial longitudinal arch support 998 is formed in base insole 957 to support the medial longitudinal arch curve of the foot. The medial longitudinal arch support 998 also provides support for the calcaneus bone of the foot in relation to the plantar fascia. The underside of the foot supports 992, 994, 996, and 998 comprise corresponding upwardly curved elevation curves 1000, 1002, 1004, and 1006 which are disposed above the inside bottom of the footwear. Each elevation curve 1000, 1002, 1004, and 1006 defines a corresponding space (not shown) with the inside bottom of the footwear such that the foot supports 992, 994, 996, and 998 may flex downwardly under a predetermined foot pressure providing a suspension effect to reduce shock and provide selected support to the wearer's foot, and providing greater comfort during walking, jogging, or running. The predetermined foot pressure of each foot support 992, 994, 996, and 998 may be controlled by using at least one base insole strength modifying feature. The base insole 958 includes a downwardly extending lateral pivot 1008 having a lowermost portion which is at a position relative to a bottom plane (not shown) of the base insole 958 preferably either slightly above or even with the bottom plane. The lateral pivot 1008 assists in guiding the foot medially to the big toe for toe-off during striding and is used to adjust supination of the foot during striding.

The metatarsal arch compression spring 959 of the eighth version insole customizing kit 958 is made of a spring wire 1010 and having opposite ends 1012, one of the ends 1012 being securable to the elevation curve 1000 below the metatarsal arch support 992. The lateral cuboid compression spring 960 is made of a spring wire 1014 and having opposite ends 1016, one of the ends 1016 being securable to the elevation curve 1002 below the lateral cuboid support 994. Spring wires 1010 and 1014 of compression springs 959 and 960 are each preferably of a diameter of between about 0.010 and 0.020 inch and the compression springs 959 and 960 preferably have between about four to six coils, respective diameters of between about three-eighths to one-half inch, and of about one-quarter inch, and fit (when slightly compressed) into the approximately one-quarter to three-eighths inch space below respective of the elevation curves 1000 and 1002. Compression springs 959 and 960 with different spring constants may be used to provide a desired amount of support for the metatarsal arch support 992 and the lateral cuboid support 994. Likewise, other types of compression springs 959 and 960 may be used such as conical springs, or non-round-coiled springs such as leaf springs, and the like. The ends 1012 and 1016 of compression springs 959 and 960 are preferably ground flat or otherwise designed to spread out the force of compression springs 959 and 960 on the base insole 957 to provide comfortable, non-bulging foot support and to minimize wear to the wearer's shoe.

The metatarsal arch compression spring 959 is securable to the elevation curve 1000 below the metatarsal arch support 992 using an attachment device of the base insole 957 comprising a center plug 1018 which extends downwardly therefrom adapted to be closely received within one end 1012 of spring 959. An annular groove 1020 is coaxially disposed about center plug 1018 with a depth of about one-half the diameter of wire 1010 and adapted to closely receive the one end 1012 of spring 959. A plurality of resilient retaining tabs 1022 extend radially outwardly from center plug 1018 which are adapted to deform sufficiently to allow wire 1010 to pass thereby and retain the one end 1012 disposed thereabout within groove 1012. The center plug 1018 extends downwardly from elevation curve 1000 a minimal distance, being about one-half the diameter of wire of 1010 plus the thickness of tabs 1022. The combination of center plug 1018 and groove 1012 are used to permit the thickness of the base insole 957 at the metatarsal arch support 992 to remain relatively thin without undue weakening by groove 1020, while firmly securing the metatarsal arch compression spring 959 to the elevation curve 1000 to back-fill the open space below the metatarsal arch support 992 and provide extra support for the metatarsal arch support 992. The opposite end 1012 of metatarsal arch compression spring 959 may be secured to the inside bottom of the footwear as described above for end caps 918 and 940.

The lateral cuboid compression spring 960 is securable to the elevation curve 1002 below the lateral cuboid support 994 using an attachment device of the base insole 957 comprising a center plug 1024, of the same design as center plug 1018, which extends downwardly therefrom adapted to be closely received within one end 1016 of spring 960. An annular groove 1026 is coaxially disposed about center plug 1024 with a depth of about one-half the diameter of wire 1014 and adapted to closely receive the one end 1016 of spring 960. A plurality of resilient retaining tabs 1028 extend radially outwardly from center plug 1024 which are adapted to deform sufficiently to allow wire 1014 to pass thereby and retain the one end 1016 disposed thereabout within groove 1026. The center plug 1024 extends downwardly from elevation curve 1002 a minimal distance of about one-half the diameter of wire of 1014 plus the thickness of tabs 1028. The combination of center plug 1024 and groove 1026 are used to permit the thickness of the base insole 957 at the lateral cuboid support 994 to remain relatively thin without undue weakening by groove 1026, while firmly securing the lateral cuboid compression spring 960 to the elevation curve 1002 to back-fill the open space below the lateral cuboid support 994 and provide extra support for the lateral cuboid support 994. The opposite end 1016 of lateral cuboid compression spring 960 may be secured to the inside bottom of the footwear as described above for end caps 918 and 940.

The medial longitudinal arch support pad 961 of the eighth version insole customizing kit 958 is made of a resilient material, preferably a generally semi-circular sheet of foam, preferably a visco-elastic foam, in a tapered configuration and having a generally straight medial edge 1030 and an arcuate, tapered lateral edge 1032. The medial longitudinal arch support pad 961 is adapted to be secured to the lower surface 412 of the upper insole 408 at the medial longitudinal arch of the foot using an adhesive attachment device in the form of an adhesive layer 1034 temporarily covered with a peel-off backing sheet 1036 to provide increased support for the medial longitudinal arch of the foot. Alternatively, other adhesive or non-adhesive attachment devices such as double-sided adhesive tape or an adhesive compound such as hardening liquid adhesive may be used. The medial longitudinal arch support pad 961 provides increased support for the medial longitudinal arch of the foot. The medial longitudinal arch support pad 961 may also be made of a more solid material if more rigid support is desired.

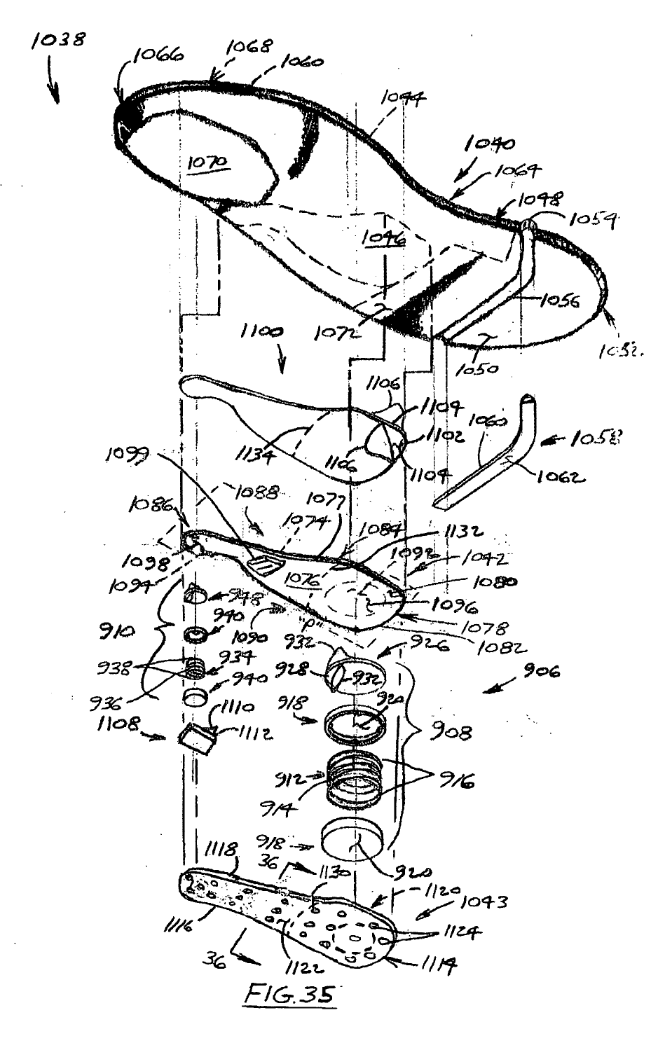

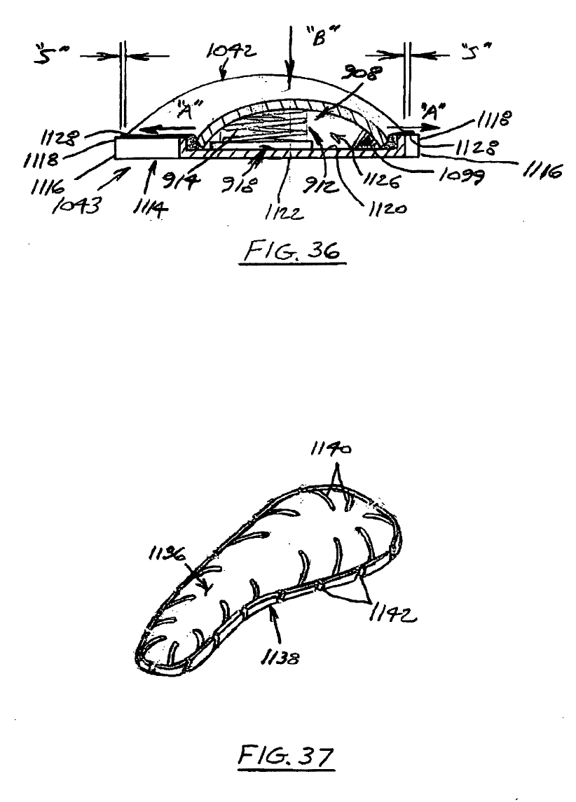

Referring to FIGS. 35 and 36, therein is shown a seventeenth embodiment user-customizable insole 1038 which includes an eighth version upper insole 1040 of full length and a base insole in the form of a first version partial base insole 1042 with an optional first version partial base support 1043 which is perforated, and is shown as used with the seventh version insole customizing kit 906.

The upper insole 1040 is relatively soft providing cushioning for the foot and shaped to fit into the footwear (not shown). The upper insole 1040 is of a flexible monolithic construction made of a flexible resilient cushioning material, such as ethylene-vinyl acetate polymer (EVA), though other similar cushioning open or closed cell foams may be used. The length of the upper insole 1040 extends from the heel to the toes of the foot. The upper insole 1040 is shaped like the outline of the sole or plantar surface of the foot, having an upper surface 1044 adapted to engage the plantar surface of the wearer's foot, and a lower surface 1046 adapted to fit to the base insole 1042. The upper insole 1040 is molded with contours to match the contours of the plantar surface of the foot and of the base insole 1042. The upper insole 1040 includes a forefoot portion 1048 positionable underneath the metatarsal bones of the wearer's foot beginning at a frontal area 1050 of the upper insole 1040 comprising a toe portion 1052. A slightly curved, integral toe grip 1054 extends generally transversely across the upper surface 1044 at the toe portion 1052 disposed adjacent a sulcus cavity of the wearer's foot. The toe grip 1054 is upwardly formed and defines a lower channel 1056. The lower channel 1056 is backfilled by a resilient toe insert 1058 affixed within the lower channel 1056 which prevents lower channel 1056 from collapsing during use. The toe insert 1058 is molded from a visco-elastic foam, having a curved upper surface 1060 and a flat lower surface 1062. Toe insert 1058 is provided to support the toe grip 1054, the toe grip 1054 and toe insert being of preselected hardnesses and resiliencies to support the toes of the wearer's fool as desired. A mid-portion 1064 of the upper insole 1040 interconnects the forefoot portion 1048 to a heel portion 1066. The heel portion 1066 is positionable under the calcaneus bone of the wearer's foot and has a concave heel cup 1068 to match the contour of the heel of the wearer's foot and a flat bottom 1070 opposite heel cup 1068 adapted to facilitate placement of heel adjustment devices thereunder. The toe portion 1052 extends forwardly as part of the frontal area 1050 of the upper insole 1040 under the phalanges of the foot to provide cushioning. The length of the upper insole 1040 is optionally of two-thirds length with a forward portion 1072 removed, as shown by the dotted line which is a trim line, a perforated line to facilitate breaking therealong, or showing a front edge if upper insole 1040 is initially manufactured as two-thirds length, which extends from the heel to behind the metatarsals.

The partial base insole 1042 includes an upper surface 1074 which is contoured to provide support to a portion of the plantar surface of the wearer's foot and a lower surface 1076 adapted to engage the inside bottom of the footwear. The partial base insole 1042 extends from behind the lateral cuboid bones to the forefoot of a wearer's foot behind the metatarsal heads of the foot and includes a peripheral edge 1077 disposed in a common plane “P”. The partial base insole 1042 includes a forefoot portion 1078 positionable underneath the metatarsal bones of the wearer's foot beginning at a frontal area 1080 of the partial base insole 1042 comprising a forward edge 1082. A mid-portion 1084 of the partial base insole 1042 interconnects the forefoot portion 1078 to a heel portion 1086, with respective medial and lateral sides 1088 and 1090 of the partial base insole 1042.

The partial base insole 1042 has a built-in orthopedic contour, including a pair of integral foot supports. An upwardly curved metatarsal arch support 1092 is formed in partial base insole 1042 to support the metatarsal bones of the foot. An upwardly curved lateral cuboid support 1094 is formed in partial base insole 1042 to support the cuboid bone of the foot. The underside of the foot supports 1092 and 1094 comprise corresponding upwardly curved elevation curves 1096 and 1098 which are disposed above the inside bottom of the footwear. Each elevation curve 1096 and 1098 defines a corresponding space (not shown) with the inside bottom of the footwear such that the foot supports 1092 and 1094 may flex downwardly under a predetermined foot pressure providing a suspension effect to reduce shock and provide selected support to the wearer's foot, and providing greater comfort during walking, jogging, or running. The predetermined foot pressure of each foot support 1092 and 1094 may be controlled by using at least one partial base insole strength modifying feature. The partial base insole 1042 may include a downwardly extending lateral pivot (not shown) to assist in guiding the foot medially to the big toe for toe-off during striding and is used to adjust supination of the foot during striding. Likewise, the partial base insole 1042 as well as all of the base insoles above, may be unfilled, partially filled, or completely backfilled with a visco-elastic material 1099 or other desired material such as below the elevation curves 1096 and 1098 to provide additional support for the metatarsal arch support 1092 and/or the lateral cuboid support 1094, or other arches of other base insoles.

The metatarsal arch spring support 908 of the seventh version insole customizing kit 906 is secured to the elevation curve 1096 below the metatarsal arch support 1092 using an adhesive attachment device such as the adhesive disk 926 of metatarsal arch spring support 908 to back-fill the open space below the metatarsal arch support 1092 and provide extra support for the metatarsal arch support 1092. Alternatively, other adhesive or non-adhesive attachment devices such as double-sided adhesive tape, an adhesive compound such as hardening liquid adhesive, or an adhesive layer temporarily covered with a peel-off backing sheet may be used. The opposite end cap 918 may be secured to the inside bottom of the footwear in a similar manner.

The lateral cuboid spring support 910 of the seventh version insole customizing kit 906 is secured to the elevation curve 1098 below the lateral cuboid support 1094 using an adhesive attachment device such as the adhesive disk 948 of lateral cuboid spring support 910 to back-fill the open space below the lateral cuboid support 1094 and provide extra support for the lateral cuboid support 1092. Alternatively, other adhesive or non-adhesive attachment devices such as double-sided adhesive tape, an adhesive compound such as hardening liquid adhesive, or an adhesive layer temporarily covered with a peel-off backing sheet may be used. The opposite end cap 940 may be secured to the inside bottom of the footwear in a similar manner.

The partial base insole 1042 with attached metatarsal arch spring support 908 and lateral cuboid spring support 910 is adapted to be secured to the lower surface 1046 of the upper insole 1040 using an adhesive attachment device in the form of an adhesive patch 1100 which is made from the same material as commercially available double sided adhesive foam core tape or similarly suitable material. Alternatively, other adhesive or non-adhesive attachment devices such as double-sided adhesive tape, an adhesive compound such as hardening liquid adhesive, or an adhesive layer temporarily covered with a peel-off backing sheet may be used. The adhesive patch 1100 is of the same shape as the partial base insole 1042 and includes a core 1102 made of a resilient sheet foam material coated with on opposite sides with respective adhesive layers 1104 temporarily covered with respective peel-off backing sheets 1106. The adhesive layers 1104 secure partial base insole 1042 with attached metatarsal arch spring support 908 and lateral cuboid spring support 910 to the to the upper insole 1040. The metatarsal arch spring support 908 and lateral cuboid spring support 910 are respectively secured to the elevation curves 1096 and 1098 below the metatarsal arch support 1092 and lateral cuboid support 1094 to provide extra support for the metatarsal arch support 1092 and the lateral cuboid support 1094.

Alternatively to the lateral cuboid spring support 910, the lateral cuboid adjustment device may be a resilient lateral cuboid support pad 1108 adapted to be secured to the elevation curve 1098 below the lateral cuboid support 1094 to provide increased support for the lateral cuboid of the foot and provide custom comfort and extra support to the wearer's foot. The lateral cuboid support pad 1108 is made a resilient material, preferably a sheet of foam rectangular in shape, preferably a visco-elastic foam, temporarily covered having an adhesive attachment device in the form of an adhesive layer 1112 temporarily covered with a peel-off backing sheet 1110 to secure it to the elevation curve 1098 below the lateral cuboid support 1094. The lateral cuboid support pad 1108 may also be affixed to the partial base insole 1042 using other adhesive or non-adhesive attachment devices such as double-sided adhesive tape, or an adhesive compound such as hardening liquid adhesive.

The first version partial base support 1043 includes a flat plate 1114 with an outer periphery 1116 of the same shape as partial base insole 1042 but slightly larger with an upstanding peripheral rim 1118. An upper surface 1120 of plate 1114 is adapted to engage the peripheral edge 1077 of partial base insole 1042 with a space “S” of between about one-thirty-second inch and one-eight inch with peripheral rim 1118. This spacing permits the resulting outward movement of partial base insole 1042, as shown at arrows “A”, without being restrained by peripheral rim 1118 as a consequence of downward flexing of the partial base insole 1042, as shown at arrow “B”, by force applied during use by the wearer's foot (not shown). A lower surface 1122 of partial base insole 1042 is adapted to engage the inside bottom of the footwear. A plurality of perforations or holes 1124 may be disposed through plate 1114 to adjust respective longitudinal and torsional rigidities thereof. Likewise, other of the strength modifying features described above for base insoles may be used. The partial base insole defines an interior chamber 1126 with the partial base support 1043. The metatarsal arch spring support 908 and the lateral cuboid spring support 910 may be disposed in interior chamber 1126 respectively below the elevation curves 1096 and 1098 to support the metatarsal arch support 1092 and the lateral cuboid support 1094 of partial base insole 1042. Likewise, the interior chamber 1126 respectively below the elevation curves 1096 and 1098 may remain empty, or may be partially or completely backfilled with a visco-elastic material (not shown) or other desired material below the elevation curves 1096 and 1098 to provide additional support for the metatarsal arch support 1092 and/or the lateral cuboid support 1094. The partial base insole 1042 is resiliently secured to the partial base support 1043 using an attachment device such as a resilient peripheral bead 1128 of conventional silicone rubber or the like which retains them together yet allows flexing therebetween.

The partial base insole 1042 and the partial base support 1043 may be of a shorter length to extend only under the metatarsal arch of the wearer's foot as shown by dotted lines 1130, 1132, and 1134 across the partial base insole 1042, partial base support 1043, and adhesive patch 1100. This is preferably done during manufacture, though trimming or breaking along perforations (not shown) provided may also be done.

Referring to FIG. 37, therein is shown a second version partial base insole 1136 with an optional second version partial base support 1138 assembled thereto. The partial base insole 1136 is the same as partial base insole 1042 except for a plurality of slots 1140 which extend inwardly from peripheral edge 1077 to adjust respective longitudinal and torsional rigidities thereof. Likewise, other of the strength modifying features described above for base insoles may be used. The partial base support 1138 is likewise the same as partial base support 1043 except for the holes 1124 being replaced by a plurality of slots 1142 which extend inwardly from outer periphery 1116 to adjust respective longitudinal and torsional rigidities thereof. Likewise, other of the strength modifying features described above for base insoles may be used. The metatarsal arch spring support 908 and the lateral cuboid spring support 910 may be disposed in interior chamber 1126, it may remain empty, or may be partially or completely backfilled with a visco-elastic material (not shown) therebetween as described above. The partial base insole 1136 is resiliently secured to the partial base support 1138 and each may be of a shorter length as described above.

A method for customizing insoles of footwear for a wearer's foot of the present invention is disclosed for the insoles of the present invention. The method comprises the steps of: 1) providing an article of footwear; 2) providing an insole adapted for placement onto a inside bottom of the footwear to support a plantar surface of the wearer's foot, the insole having a cushioning upper insole with a forefoot portion, a mid-portion, and a heel portion, and a base insole shaped to fit to the footwear having a forefoot portion, a mid-portion, and a heel portion with a heel cup; 3) providing a plurality of insole adjustment devices each adapted to correct or accommodate foot position abnormality or state and support the wearer's foot within the footwear in a corrected position; 4) selecting at least one of the insole adjustment devices to correct an abnormal foot position of the wearer's foot and support the foot in the corrected position; 5) placing the selected adjustment devices into respective desired corrective positions to the insole; and 6) inserting the insole with the selected adjustment devices in the corrective positions into the article of footwear.

In a preferred method the step of placing the selected adjustment device includes securing the selected adjustment devices to the insole. The step of placing the adjustment device preferably includes securing the selected adjustment devices to the insole using an adhesive layer of the adjustment devices which are uncovered by removing a protective cover sheet or securing the selected adjustment devices to the insole using a double-sided adhesive tape. The step of providing a plurality of insole adjustment devices preferably comprises providing at least one adjustment device selected from the group consisting of first metatarsal head boots, forefoot medial wedges, forefoot lateral wedges, medial longitudinal wedges, lateral longitudinal wedges, bi-planar rear posts, heel lifts, metatarsal arch support pads, longitudinal arch support pads, heel medial half-wedges, heel lateral half wedges, heel full wedges, contoured heel pads, pivot pads, undersurface pads, heel gel pads, and spring insole adjustment devices. The step of providing an insole preferably comprises providing an insole wherein the mid-portions of the upper and base insoles are affixed together allowing lifting of the forefoot and heel portions of the upper insole away from the base insole to allow placement of the adjustment devices of an intrinsic type between the upper and base insoles.

Many changes to the present invention are possible while staying within the same inventive concept. For example, the insoles may be of laminated construction made of layers sheet foam or other suitable materials which are adhesively or otherwise bonded together, or of homogeneous construction from a single piece of foam or other suitable material. The intrinsic and extrinsic insole adjustment devices and the toe grips are shown adhered to the upper and base insoles using an adhesive layer covered by a removable backing sheet. However, other attachment devices for retaining the insole adjustment devices may be utilized such as double sided adhesive tape, pairs of interlockable hook and loop fastener material such as sold under the trade name VELCRO™. The adjustment devices may be supplied as separate items or attached together such as by tabs for later disattachment, particularly for kit form. The insole adjustment devices may also remain unattached to the upper and base insoles or the insole and the footwear, being retained therebetween by foot pressure. The metatarsal arch adjustment device and the lateral cuboid adjustment device which utilize compression springs may be adapted for use with the other arch supports of the base insoles or for use directly with the upper insoles alone or above the base insoles. Additionally, the particular configurations, combinations, and use of the intrinsic and extrinsic insole adjustments are only exemplary of the virtually limitless diversity thereof to suit the particular wearer's foot.

Whereas this invention is here illustrated and described with reference to embodiments thereof presently contemplated as the best mode of carrying out such invention in actual practice, it is to be understood that various changes may be made in adapting the invention to different embodiments without departing from the broader inventive concepts disclosed herein and comprehended by the claims that follow.

I claim:

1. A user-customizable insole for placement onto an inside bottom of footwear to support the plantar surface of a wearer's foot, comprising:
an upper insole having an upper surface adapted to engage at least a portion of the plantar surface of the wearer's foot and a lower surface; and
a base insole having an upper surface adapted to engage said lower surface of said upper insole and a lower surface adapted to engage the inside bottom of the footwear, at least a portion of said upper insole being separable from said base insole to allow introduction of insole adjustment devices therebetween to provide custom comfort and support to the wearer's foot.

2. The insole of claim 1, wherein the upper and base insoles include respective forefoot portions positionable underneath metatarsal bones of the wearer's foot, respective heel portions positionable underneath the calcaneus bone of the wearer's foot, and respective mid-portions which interconnect said forefoot and heel portions.

3. The insole of claim 1, wherein the upper and base insoles are each of a length chosen from the group consisting of two-thirds length where a forefoot portion begins at a forward edge thereof disposed adjacent a sulcus cavity of the wearer's foot, two-thirds length with a first metatarsal head extension which extends from said forward edge under the first metatarsal head of the wearer's foot, and full length with a toe portion positionable underneath phalanges of the wearer's foot with about a front most one-third being of a different thickness than a remaining two-thirds thereof.

4. The insole of claim 1, wherein the upper insole is of a construction chosen from the group consisting of a monolithic construction comprising a body made of a single flexible resilient cushioning material, a flexible sandwiched construction comprising a flexible cloth top sheet bonded to a middle layer made of a resilient cushioning material, a cloth construction comprising a flexible cloth sheet with the bottom surface covered with an adhesive layer, and a semi-rigid sandwiched construction comprising a flexible cloth sheet bonded to a semi-rigid contoured foam body.

5. The insole of claim 1, wherein the base insole includes at least one integral foot support chosen from the group consisting of a metatarsal arch support, a lateral cuboid support, a medial longitudinal arch support, a lateral arch support, and a medial longitudinal ligament support.

6. The insole of claim 5, wherein the base insole includes a metatarsal arch support, and a forefoot portion includes a narrow flat support lip disposed from a forward edge rearwardly into said metatarsal arch support.

7. The insole of claim 5, wherein the forward edge of the base insole is feathered.

8. The insole of claim 5, wherein the base insole includes a metatarsal arch support and a forefoot portion includes a at least one slot therethrough which extend from a forward edge rearwardly into the metatarsal arch support.

9. The insole of claim 8, wherein there are four slots which are disposed so as to be between respective first, second, third, fourth, and fifth metatarsal bones of the wearer's foot.

10. The insole of claim 1, wherein a heel portion of the base insole includes a concave heel cup having a flat bottom, said heel cup being of a depth sufficient to accept at least one intrinsic heel adjustment device therein.

11. The insole of claim 10, wherein a heel portion of the upper insole has a bottom of a type chosen from the group consisting of flat, rounded, and rounded flat adapted to facilitate placement of intrinsic heel adjustment devices in the heel cup of the base insole.

12. The insole of claim 1, wherein the base insole includes a tapered perimeter wall which extends around a heel portion and forwardly along respective medial and lateral sides of said base insole defining a heel cup, said support wall tapering away at about a forefoot portion of said base insole.

13. The insole of claim 1, wherein the base insole is relatively thin and of a generally uniform thickness, said base insole having at least one upwardly curved integral foot support with a corresponding upwardly curved elevation curve therebelow, said foot support being chosen from the group consisting of a metatarsal arch support, a lateral cuboid support, a medial longitudinal arch support, and a lateral arch support, each elevation curve defining a corresponding space with the inside bottom of the footwear such that said foot support may flex downwardly under a predetermined foot pressure to reduce shock and provide selected support to the wearer's foot.

14. The insole of claim 13, wherein the predetermined foot pressure of each foot support is controlled by using at least one base insole strength modifying feature chosen from the group consisting of perforations, open-ended slots, closed-ended slots, thinned base insole material, adding external material to selectively strengthen said slots, regional changes to mechanical properties of material of the base insole, and selective back-filling of the partial base insole.

15. The insole of claim 1, wherein the base insole includes at least one reinforcing rib which extends along the lower surface to stiffen said base insole.

16. The insole of claim 1, wherein the base insole includes a first metatarsal head extension that is between about 0.010 and 0.095 inch thick.

17. The insole of claim 1, wherein the base insole includes an integrally molded heel support which extends downwardly from a heel portion thereof, said heel support having a flat bottom adapted to engage the inside bottom of the footwear to stabilize the heel of the wearer's foot within the footwear.

18. The insole of claim 1, wherein the base insole includes a separately molded heel support having an upper surface adapted to be affixed to a heel portion of said base insole, said heel support which extends downwardly from said heel portion and has a flat bottom adapted to engage the inside bottom of the footwear to stabilize the heel of the wearer's foot within the footwear.

19. The insole of claim 1, wherein the lower surface of the base insole is adapted to receive at least one insole adjustment device to provide custom comfort and support to the wearer's foot chosen from the group consisting of a metatarsal arch support pad, heel wedges, and spring insole adjustment devices.

20. The insole of claim 1, wherein the base insole includes a U-shaped heel support which is affixed to a heel portion of said base insole extending downwardly from said heel portion and forwardly along respective medial and lateral sides thereof, said heel support forming an open space under the lower surface of said base insole to permit said base insole to flex downwardly into said space to cushion heel impacts, said heel support having a flat bottom adapted to engage the inside bottom of the footwear to stabilize the heel of the wearer's foot within the footwear.

21. The insole of claim 20, wherein the open space under the lower surface of the base insole is filled with a resilient filler material.

22. The insole of claim 1, wherein the base insole includes a resilient heel support affixed to a heel portion of said base insole, said heel support which extends downwardly from said heel portion, said heel support having a flat bottom adapted to engage the inside bottom of the footwear to resiliently stabilize the heel of the wearer's foot within the footwear.

23. The insole of claim 1, wherein the base insole includes a downwardly extending lateral pivot of a type chosen from the group consisting of uncovered and covered by a resilient pivot pad.

24. The insole of claim 1, wherein at least one of the upper insole and the base insole includes positioning indicia thereon to facilitate placement of adjustment devices thereon.

25. The insole of claim 1, wherein the upper insole includes an integral toe grip which extends laterally across the upper surface disposed adjacent a sulcus cavity of the wearer's foot.

26. The insole of claim 1, wherein a mid-portion of the upper insole is affixed to a mid-portion of the base insole such that a heel portion and a forefoot portion of said upper insole may be raised from said base insole to add and remove adjustment devices.

27. The insole of claim 1, wherein a heel portion of the upper insole includes a concave heel cup.

28. A user-customizable insole for placement onto an inside bottom of footwear to support a plantar surface of a wearer's foot, comprising:
an upper insole having an upper surface adapted to engage at least a portion of the plantar surface of the wearer's foot and a lower surface;
a base insole having an upper surface adapted to engage said lower surface of said upper insole and a lower surface adapted to engage the inside bottom of the footwear, at least a portion of said upper insole being separable from said base insole to allow introduction of insole adjustment devices therebetween to provide custom comfort and support to the wearer's foot; and
at least one insole adjustment device adapted to be used with said insole for adjusting the comfort and support of the wearer's foot within the foot wear.

29. The insole of claim 28, wherein the upper and base insoles are each of a length chosen from the group consisting of two-thirds length where a forefoot portion begins at a forward edge thereof disposed adjacent the sulcus cavity of the wearer's foot, two-thirds length with a first metatarsal head extension which extends from said forward edge under the first metatarsal head of the wearer's foot, and full length with a toe portion positionable underneath phalanges of the wearer's foot with about a front most one-third being of a different thickness than a remaining two-thirds thereof.

30. The insole of claim 28, wherein the base insole includes the metatarsal arch support and a forefoot portion thereof includes a at least one slot therethrough which extend from a forward edge rearwardly into said metatarsal arch support.

31. The insole of claim 28, wherein the base insole is relatively thin and of a generally uniform thickness, said base insole having at least one upwardly curved integral foot support with a corresponding upwardly curved elevation curve therebelow, said foot support being chosen from the group consisting of a metatarsal arch support, a lateral cuboid support, a medial longitudinal arch support, and a lateral arch support, each elevation curve defining a corresponding space with the inside bottom of the footwear such that said foot support may flex downwardly under a predetermined foot pressure to reduce shock and provide selected support to the wearer's foot.

32. The insole of claim 31, wherein the predetermined foot pressure of each foot support is controlled by using at least one base insole strength modifying feature chosen from the group consisting of perforations, open-ended slots, closed-ended slots, thinned base insole material, adding external material to selectively strengthen said slots, regional changes to mechanical properties of material of the base insole, and selective back-filling of the partial base insole.

33. The insole of claim 28, where in the base insole includes a heel support which extends downwardly from a heel portion thereof, said heel support having a flat bottom adapted to engage the inside bottom of the footwear to stabilize the heel of the wearer's foot within the footwear.

34. The insole of claim 28, wherein the base insole includes a downwardly extending lateral pivot of a type chosen from the group consisting of uncovered and covered by a resilient pivot pad.

35. The insole of claim 28, wherein the upper insole is completely removable from the base insole to allow the insole adjustment devices to be placed on said base insole and the upper insole placed thereon.

36. An insole customizing kit for user-customizable insoles of the type having at least a partially separable upper and base insole, comprising at least one insole adjustment device adapted to be used with said insole for adjusting the comfort and support of the wearer's foot within the foot wear.

37. The customizing kit of claim 36, further comprising at least one insole adjustment device chosen from the group consisting of a first metatarsal head extension boot, a forefoot medial wedge, a forefoot lateral wedge, an elongate medial longitudinal wedge, an elongate lateral longitudinal wedge, a bi-planar rear post, a heel lift, a resilient metatarsal arch support pad, a resilient longitudinal arch support pad, a medial half-heel wedge, a lateral half-heel wedge, a full-width heel wedge, a resilient contoured foam heel pad, a resilient pivot pad, a resilient undersurface pad, a contoured gel heel pad, a custom positionable toe grip, and spring insole adjustment devices.

38. The customizing kit of claim 36, wherein each adjustment device includes an attachment device chosen from the group consisting of a peel-off backing sheet which exposes an adhesive layer on said adjustment device, an adhesive layer on said adjustment device, double-sided adhesive tape, and other attachment devices to secure said adjustment device at least to one of the upper insole and the base insole.

39. The customizing kit of claim 37, including the toe grip which comprises a slightly curved elongate body adapted to extend laterally across a toe portion of the upper insole adjacent a sulcus cavity of the wearer's foot, said toe grip having at least one upper peel-off backing sheet which exposes a corresponding upper adhesive layer adapted to temporarily secure said toe grip to an underside of the toes at the sulcus cavity of the foot, and at least one lower peel-off backing sheet which exposes a lower adhesive layer to semi-permanently secure said toe grip to the toe portion, said upper adhesive layer having a lower affinity to the underside of the toes than said lower adhesive layer has to the upper surface of the insole, said toe grip being attached and held in place under the toes until the foot is placed onto the insole such that said toe grip attaches to the upper surface in an exact position when body weight is applied and releases from the toes when the foot is removed therefrom.

40. The customizing kit of claim 39, wherein a combined surface area of said body covered by said upper adhesive layers is smaller than a combined surface area of said body covered by said lower adhesive layers to provide the greater affinity.

41. The customizing kit of claim 39, wherein the body of the toe grip comprises a resilient body onto which respective upper and lower cloth sheets are bonded.

42. A method for customizing insoles of footwear to provide comfort and support to the plantar surface of a wearer's foot, comprising the steps of:
providing a suitably sized article of footwear having an inside bottom;
providing a suitably sized user-customizable insole adapted for placement onto the inside bottom of the footwear to support the plantar surface of the wearer's foot;
providing a plurality of insole adjustment devices each adapted to be used with the insole for adjusting the comfort and support of the wearer's foot within the foot wear;
selecting at least one of the insole adjustment devices for adjusting the comfort and support of the wearer's foot within the foot wear;
placing the selected adjustment devices into appropriate positions against the insole; and
inserting the insole with the selected adjustment devices in the appropriate positions into the article of footwear.

43. The method of claim 42, wherein the step of placing the selected adjustment device includes securing the selected adjustment devices to the insole.

44. The method of claim 43, wherein the step of placing the selected adjustment devices includes securing the selected adjustment devices to the insole by removing respective cover sheets to expose an adhesive layer on the selected insole adjustment devices and adhering them to the insole.

45. The method of claim 43, wherein the step of placing the selected adjustment devices includes securing the selected adjustment devices to the insole using double-sided adhesive tape.

46. The method of claim 42, wherein the step of providing an insole comprises providing an insole having at least partially separable upper and base insoles wherein respective mid-portions of the upper and base insoles are affixed together allowing lifting of the forefoot and heel portions of the upper insole away from the base insole for placement of selected adjustment devices between the upper and base insoles.

47. The method of claim 46, wherein the step of providing a plurality of insole adjustment devices comprises providing at least one adjustment a device selected from the group consisting of first metatarsal head boots, forefoot medial wedges, forefoot lateral wedges, medial longitudinal wedges, medial lateral wedges, bi-planar rear posts, heel lifts, resilient metatarsal arch support pads, resilient longitudinal arch support pads, heel medial half-wedges, heel lateral half wedges, heel fall wedges, resilient contoured heel pads, resilient pivot pads, resilient undersurface pads, heel gel pads, and spring insole adjustment devices.

48. A user-customizable insole for placement onto an inside bottom of footwear to support the plantar surface of a wearer's foot, comprising:
a body having an upper surface adapted to engage at least a portion of the plantar surface of the wearer's foot and a lower surface adapted to engage the inside bottom of the footwear, said body being of a type chosen from the group consisting of non-separable unitary construction and a separable construction comprised of at least partially separable upper and base insoles adapted to allow introduction of insole adjustment devices therebetween to provide custom comfort and support to the wearer's foot; and
at least one insole adjustment device adapted to be used with said insole for adjusting the comfort and support of the wearer's foot within the foot wear.

49. The insole of claim 48, wherein each insole adjustment device is chosen from the group consisting of a first metatarsal head extension boot, a forefoot medial wedge, a forefoot lateral wedge, an elongate medial longitudinal wedge, an elongate lateral longitudinal wedge, a bi-planar rear post, a heel lift, a resilient metatarsal arch support pad, a resilient longitudinal arch support pad, a medial half-heel wedge, a lateral half-heel wedge, a full-width heel wedge, a resilient contoured foam heel pad, a resilient pivot pad, a resilient undersurface pad, a contoured gel heel pad, a custom positionable toe grip, and spring insole adjustment devices.

50. The insole of claim 48, wherein a heel portion of the upper insole has a bottom of a type chosen from the group consisting of flat, rounded, and rounded flat adapted to facilitate placement of intrinsic heel adjustment devices in the heel cup of the base insole.

51. The insole of claim 48, wherein the base insole includes a downwardly extending lateral pivot of a type chosen from the group consisting of uncovered and covered by a resilient pivot pad.

52. A user-customizable insole for placement onto an inside bottom of footwear to support the plantar surface of a wearer's foot, comprising:
an upper insole having an upper surface adapted to engage at least a portion of the plantar surface of the wearer's foot and a lower surface; and
a base insole having an upper surface adapted to engage said lower surface of said upper insole, a lower surface adapted to engage the inside bottom of the footwear, said base insole being of two-thirds length wherein a forefoot portion begins at a forward edge thereof disposed adjacent a sulcus cavity of the wearer's foot with a first metatarsal head extension which extends from said forward edge under a first metatarsal head of the wearer's foot, at least a portion of said upper insole being separable from said base insole to allow introduction of insole adjustment devices therebetween to provide custom comfort and support to the wearer's foot.

53. The insole of claim 52, further comprising:
at least one insole adjustment device adapted to be used with the insole for adjusting the comfort and support of the wearer's foot within the foot wear; and
wherein each insole adjustment device is chosen from the group consisting of a first metatarsal head extension boot, a forefoot medial wedge, a forefoot lateral wedge, an elongate medial longitudinal wedge, an elongate lateral longitudinal wedge, a bi-planar rear post, a heel lift, a resilient metatarsal arch support pad, a resilient longitudinal arch support pad, a medial half-heel wedge, a lateral half-heel wedge, a full-width heel wedge, a resilient contoured foam heel pad, a resilient pivot pad, a resilient undersurface pad, a contoured gel heel pad, a custom positionable toe grip, and spring insole adjustment devices.

54. The insole of claim 52, wherein a heel portion of the upper insole has a bottom of a type chosen from the group consisting of flat, rounded, and rounded flat adapted to facilitate placement of intrinsic heel adjustment devices in the heel cup of the base insole.

55. The insole of claim 52, wherein the base insole includes a downwardly extending lateral pivot of a type chosen from the group consisting of uncovered and covered by a resilient pivot pad.

56. A user-customizable insole for placement onto an inside bottom of footwear to support the plantar surface of a wearer's foot, comprising:
a body having an upper surface adapted to engage at least a portion of the plantar surface of the wearer's foot and a lower surface adapted to engage the inside bottom of the footwear, said body being adapted to allow introduction of insole adjustment devices therebetween to provide custom comfort and support to the wearer's foot; and
a metatarsal arch support pad made of a resilient material adapted to support a metatarsal arch of the wearer's foot.

57. The insole of claim 56, wherein the resilient material comprises a visco-elastic material.

58. The insole of claim 57, wherein the body comprises:
an upper insole having the upper surface adapted to engage at least a portion of the plantar surface of the wearer's foot and a lower surface;
a base insole having an upper surface adapted to engage said lower surface of said upper insole and the lower surface adapted to engage the inside bottom of the footwear, at least a portion of said upper insole being separable from said base insole to allow introduction of insole adjustment devices therebetween to provide custom comfort and support to the wearer's foot; and
wherein said base insole is relatively thin and of a generally uniform thickness, said base insole having an upwardly curved integral metatarsal arch support with a corresponding upwardly curved elevation curve therebelow defining a concave space with the inside bottom of the footwear such that said metatarsal arch support may flex downwardly under a predetermined foot pressure to reduce shock and provide selected support to the wearer's foot, said space being adapted to selectively receive the metatarsal arch support pad to increase metatarsal support.

59. The insole of claim 58, wherein the predetermined foot pressure of the metatarsal arch support is controlled by using at least one base insole strength modifying feature chosen from the group consisting of perforations, open-ended slots, closed-ended slots, thinned base insole material, adding external material to selectively strengthen said slots, regional changes to mechanical properties of material of the base insole, and selective back-filling of the partial base insole.

60. A user-customizable insole for placement onto an inside bottom of footwear to support the plantar surface of a wearer's foot, comprising:
an upper insole having an upper surface adapted to engage at least a portion of the plantar surface of the wearer's foot and a lower surface;
a base insole having an upper surface adapted to engage said lower surface of said upper insole and a lower surface adapted to engage the inside bottom of the footwear, said base insole having a built-in orthopedic contour which includes at least one upwardly curved integral foot support chosen from the group consisting of a metatarsal arch support adapted to support metatarsal bones of the wearer's foot and a lateral cuboid support adapted to support a cuboid bone of the wearer's foot, an underside of each foot support comprising an upwardly curved elevation curve; and
at least one insole adjustment device adapted to be disposed against a corresponding one of said elevation curves below one of said metatarsal arch support and a lateral cuboid support, said at least one insole adjustment device being chosen from the group consisting of a metatarsal arch adjustment device and a lateral cuboid adjustment device to provide custom comfort and support to the wearer's foot.

61. The insole of claim 60, wherein the metatarsal arch adjustment device comprises a metatarsal arch spring support which includes a compression spring made of a spring wire and has opposite ends, one of said ends being securable to the elevation curve to back-fill the open space below the metatarsal arch support and provide extra support for the metatarsal arch support, and the lateral cuboid adjustment device comprises a lateral cuboid spring support which includes a compression spring made of a spring wire and having opposite ends, one of said ends being securable to the elevation curve to back-fill the open space below the lateral cuboid support and provide extra support for the lateral cuboid support.

62. The insole of claim 61, wherein the one of the ends of each compression spring is securable to respective of the elevation curves using an adhesive attachment device chosen from the group consisting of double-sided adhesive tape and an adhesive compound.

63. The insole of claim 61, wherein there are both the metatarsal arch spring support and the lateral cuboid spring support.

64. The insole of claim 61, wherein any metatarsal arch spring support provided further comprises at least one end cap adapted to be affixed to the one end of the compression spring thereof and securable to the elevation curve below the metatarsal arch support, and any lateral cuboid spring support provided further comprises at least one end cap adapted to be affixed to the one end of the compression spring thereof and securable to the elevation curve below the lateral cuboid support

65. The insole of claim 64, wherein any end caps of the metatarsal arch spring support and the lateral cuboid spring support snap-fit to the one end of the compression spring thereof, each end cap including a circular end wall, a dependent circular peripheral wall adapted to closely fit about respective of said one ends, and a plurality of resilient retaining tabs which extend radially inwardly from respective of said peripheral walls opposite said end wall which are adapted to deform sufficiently to allow the wire of said compression spring associated therewith to pass thereby and retain said one of said ends disposed within said peripheral wall.

66. The insole of claim 64, wherein each metatarsal arch spring support and lateral cuboid spring support provided includes a pair of the end caps adapted to be affixed to opposite ends of the compression spring thereof.

67. The insole of claim 64, wherein the end caps are securable to the elevation curves using respective adhesive disks each of which comprise a core made of a resilient foam material coated on opposite sides with respective adhesive layers.

68. The insole of claim 60, wherein the upper insole is of a length chosen from the group consisting of full length and two-thirds length, and the base insole is of two-thirds length of a configuration chosen from the group consisting of with and without a first metatarsal head extension adapted to support a first metatarsal head of the wearer's foot.

69. The insole of claim 68, wherein the upper insole is of full length and adapted to be changed to two-thirds length by including a trim line of a type chosen from the group consisting of a molded trim line molded into said upper insole, a printed trim line printed onto said upper insole, and a perforated trim line molded into said upper insole and adapted to facilitate breaking therealong.

70. The insole of claim 60, wherein the base insole includes a downwardly extending lateral pivot.

71. The insole of claim 60, wherein the metatarsal arch adjustment device comprises a metatarsal arch spring support which includes a compression spring made of a spring wire and has opposite ends, one of said ends being securable to the elevation curve to back-fill the open space below the metatarsal arch support and provide extra support for the metatarsal arch support, the lateral cuboid adjustment device comprises a lateral cuboid spring support which includes a compression spring made of a spring wire and having opposite ends, one of said ends being securable to the elevation curve to back-fill the open space below the lateral cuboid support and provide extra support for the lateral cuboid support, and wherein said metatarsal arch spring support and said lateral cuboid spring support are securable to respective of said elevation curves using an attachment device of the base insole chosen from the group consisting of a center plug, an annular groove, and a combination thereof.

72. The insole of claim 71, wherein each attachment device includes a plurality of resilient retaining tabs which extend radially therefrom adapted to deform sufficiently to allow said wire of respective of the compression springs to pass thereby and retain the thereof.

73. The insole of claim 60, further comprising a medial longitudinal arch support pad made of a resilient material in a tapered configuration adapted to be secured to the lower surface of the upper insole using an adhesive attachment device chosen from the group consisting of double-sided adhesive tape, an adhesive compound, and an adhesive layer temporarily covered with a peel-off backing sheet to provide increased support for the medial longitudinal arch of the foot.

74. The insole of claim 60, wherein the base insole comprises a partial base insole.

75. The insole of claim 74, wherein the partial base insole includes both the metatarsal arch support and the lateral cuboid support.

76. The insole of claim 74, wherein the partial base insole is adapted to be secured to the lower surface of the upper insole using an adhesive attachment device chosen from the group consisting of double-sided adhesive tape, an adhesive compound, and an adhesive layer temporarily covered with a peel-off backing sheet, and an adhesive patch comprising a core made of a resilient foam material coated on opposite sides with respective adhesive layers temporarily covered with respective peel-off backing sheets.

77. The insole of claim 74, wherein the partial base insole includes at least one strength modifying feature chosen from the group consisting of perforations, open-ended slots, closed-ended slots, thinned partial base insole material, adding external material to selectively strengthen said slots, regional changes to mechanical properties of material of the partial base insole, and selective back-filling of the partial base insole with a visco-elastic material.

78. The insole of claim 74, further comprising a partial base support which includes a generally flat plate with an outer periphery of same shape as the partial base insole but slightly larger than a peripheral edge of said partial base insole, and further including an upstanding peripheral rim, an upper surface adapted to engage said peripheral edge which is spaced slightly inwardly of said peripheral rim to permit flexing of the partial base insole and partial base support, a lower surface adapted to engage the inside bottom of the footwear, said partial base insole is resiliently secured to the partial base support using an attachment device, and an interior chamber being defined between said partial base insole and said partial base support.

79. The insole of claim 78, wherein the attachment device comprises a resilient peripheral bead which interconnects the peripheral edge of the partial base insole to at least one of the upper surface and the peripheral rim of he partial base support.

80. The insole of claim 78, wherein the peripheral edge of the partial base insole is disposed in a common plane, and the partial base support is flat.

81. The insole of claim 78, wherein at least one of the partial base insole and the partial base support include at least one strength modifying feature chosen from the group consisting of perforations, open-ended slots, closed-ended slots, thinned material, adding external material to selectively strengthen said slots, regional changes to mechanical properties of said material, and selective back-filling of the partial base insole with a visco-elastic material.

82. The insole of claim 78, further comprising at least one arch support device chosen from the group consisting of a metatarsal arch spring support, a lateral cuboid spring support, a metatarsal arch support pad, a lateral cuboid arch support pad, partial backfilling with a visco-elastic material, and completely backfilled with a visco-elastic material disposed within the interior chamber below respective of the elevation curves to support the metatarsal arch support and the lateral cuboid support.

83. The insole of claim 78, wherein the partial base insole includes both the metatarsal arch support and the lateral cuboid support.

84. The insole of claim 60, wherein the base insole includes the lateral cuboid support and including the lateral cuboid adjustment device comprising a resilient lateral cuboid support pad adapted to be secured to the elevation curve below the lateral cuboid support and provide extra support for the lateral cuboid support using an adhesive attachment device chosen from the group consisting of double-sided adhesive tape, an adhesive compound, and an adhesive layer temporarily covered with a peel-off backing sheet to provide increased support for the lateral cuboid of the foot to provide custom comfort and support to the wearer's foot.

85. The insole of claim 84, wherein the lateral cuboid support pad is substantially rectangular in shape.

86. A user-customizable insole for placement onto an inside bottom of footwear to support the plantar surface of a wearer's foot, comprising:
an upper insole having an upper surface adapted to engage at least a portion of the plantar surface of the wearer's foot and a lower surface, and an integral toe grip which extends generally transversely across said upper surface disposed adjacent a sulcus cavity of the wearer's foot, being upwardly formed and defining a lower channel, said lower channel being filled by a resilient toe insert affixed within said lower channel, said toe grip being of a preselected hardness and resiliency to support the toes of the wearer's foot; and
a base insole having an upper surface adapted to engage said lower surface of said upper insole and a lower surface adapted to engage the inside bottom of the footwear, said base insole having a built-in orthopedic contour to provide comfort and support to the wearer's foot.

87. A user-customizable insole for placement onto an inside bottom of footwear to support the plantar surface of a wearer's foot, comprising:
an upper insole having an upper surface adapted to engage at least a portion of the plantar surface of the wearer's foot and a lower surface;
a base insole having an upper surface adapted to engage said lower surface of said upper insole and a lower surface adapted to engage the inside bottom of the footwear, said base insole having a built-in orthopedic contour which includes at least one upwardly curved integral foot support chosen from the group consisting of a metatarsal arch support adapted to support metatarsal bones of the wearer's foot and a lateral cuboid support adapted to support a cuboid bone of the wearer's foot, an underside of each foot support comprising an upwardly curved elevation curve; and
at least one insole adjustment device adapted to be disposed against a corresponding one of said elevation curves below one of said metatarsal arch support and a lateral cuboid support, said at least one insole adjustment device being chosen from the group consisting of a metatarsal arch adjustment device and a lateral cuboid adjustment device to provide custom comfort and support to the wearer's foot, said metatarsal arch adjustment device comprising a metatarsal arch spring support which includes a compression spring made of a spring wire and has opposite ends, one of said ends being securable to said elevation curve to back-fill the open space below said metatarsal arch support and provide extra support for said metatarsal arch support, and said lateral cuboid adjustment device comprising a lateral cuboid spring support which includes a compression spring made of a spring wire and having opposite ends, one of said ends being securable to said elevation curve to back-fill the open space below said lateral cuboid support and provide extra support for said lateral cuboid support, said one of said ends of each compression spring of said metatarsal arch spring support and said lateral cuboid spring support being securable to respective of said elevation curves using an attachment device chosen from the group consisting of a center plug of said base insole with retaining tabs, an annular groove of said base insole with retaining tabs, a combination of said center plug and said annular groove of said base insole, double-sided adhesive tape, an adhesive compound, and an adhesive disk which includes a core made of a resilient foam material coated on opposite sides with respective adhesive layers.

88. The insole of claim 87, wherein there are both the metatarsal arch spring support and the lateral cuboid spring support.

89. The insole of claim 87, wherein the base insole comprises a partial base insole adapted to be secured to the lower surface of the upper insole using an adhesive attachment device chosen from the group consisting of double-sided adhesive tape, an adhesive compound, and an adhesive layer temporarily covered with a peel-off backing sheet, and an adhesive patch comprising a core made of a resilient foam material coated on opposite sides with respective adhesive layers temporarily covered with respective peel-off backing sheets.

90. The insole of claim 87, wherein any metatarsal arch spring support provided further comprises at least one end cap adapted to be affixed to the one end of the compression spring thereof and securable to the elevation curve below the metatarsal arch support, and any lateral cuboid spring support provided further comprises at least one end cap adapted to be affixed to the one end of the compression spring thereof and securable to the elevation curve below the lateral cuboid support

91. The insole of claim 90, wherein each metatarsal arch spring support and lateral cuboid spring support provided includes a pair of the end caps adapted to be affixed to opposite ends of the compression spring thereof.

92. The insole of claim 87, wherein the upper insole is of a length chosen from the group consisting of full length and two-thirds length, and the base insole is of two-thirds length of a configuration chosen from the group consisting of with and without a first metatarsal head extension adapted to support a first metatarsal head of the wearer's foot.
